(19) United States
(12) Patent Application Publication
(10) Pub. No.: US 2022/0306760 A1
(43) Pub. Date: Sep. 29, 2022

(54) IGM GLYCOVARIANTS

(71) Applicant: IGM Biosciences, Inc., Mountain View, CA (US)

(72) Inventors: Bruce KEYT, Hillsborough, CA (US); Dean NG, San Francisco, CA (US); Ramesh BALIGA, Redwood City, CA (US)

(21) Appl. No.: 17/637,349

(22) PCT Filed: Aug. 21, 2020
(86) PCT No.: PCT/US2020/047495

§ 371 Date: Feb. 22, 2022

Related U.S. Application Data
(60) Provisional application No. 62/891,263, filed on Aug. 23, 2019.

(51) Int. Cl. C07K 16/28

(52) U.S. Cl. CPC C07K 16/2887; C07K 16/2809; C07K 2317/41; C07K 2317/51; C07K 2317/622; C07K 2317/734; C07K 2317/31

(57) ABSTRACT

This disclosure provides an isolated IgM-derived binding molecule, e.g., an IgM antibody, IgM-like antibody, or other IgM-derived binding molecule, including at least one variant IgM-derived heavy chain, where the at least one variant IgM-derived heavy chain includes a variant IgM heavy chain constant region associated with a binding domain that specifically binds to a target, where at least one asparagine (N)-linked glycosylation motif of the variant IgM heavy chain constant region is mutated to prevent glycosylation at that motif, and/or at least one N-linked glycosylation motif is introduced into the variant IgM heavy chain.

### CROSS-REFERENCE TO RELATED APPLICATIONS

This application claims the benefit of U.S. Provisional Patent Application Ser. No. 62/891,263, filed Aug. 23, 2019, which is incorporated herein by reference in its entirety.

### SEQUENCE LISTING

The instant application contains a Sequence Listing which has been submitted electronically in ASCII format and is hereby incorporated by reference in its entirety. The ASCII copy was created on Aug. 21, 2020, is named 026WO1-Sequence-Listing, and is 166,573 bytes in size.

### BACKGROUND

Antibodies and antibody-like molecules that can multimerize, such as IgA and IgM antibodies, have emerged as promising drug candidates in the fields of, e.g., immuno-oncology and infectious diseases allowing for improved specificity, improved avidity, and the ability to bind to multiple binding targets. See, e.g., U.S. Pat. Nos. 9,951,134 and 9,938,347, and PCT Publication Nos. WO 2016/141303, WO 2016/154593, WO 2016/168758, WO 2017/059387, WO 2017 059380, WO 2018/017888, WO 2018/017763, WO 2018/017889, and WO 2018/017761, the contents of which are incorporated herein by reference in their entireties.

The pharmacokinetics (PK) and pharmacodynamics (PD) of multivalent antibodies are complex and depend on the structure of the monoclonal antibody both translationally and post-translationally, as well as the physiological system that it targets. Moreover, different antibody classes are typically processed within a subject via different cellular and physiological systems. For example, the IgG antibody class has a serum half-life of 20 days, whereas the half-lives for IgM and IgA antibodies are only about 5-8 days. Brekke, O H., and I. Sandlie, Nature Reviews Drug Discovery 2: 52-62 (2003).

One of the key determinants of PK of an antibody or other biotherapeutic is its level and type of glycosylation (Higel, F. et al. Eur. J. Pharm. Biopharm. 139:123-131 (2019)). Sugar moieties and their derivatives covalently linked to specific residues on an antibody can determine how they are recognized by receptors such as asialo-glycoprotein (ASGP) receptor, which in turn determines how quickly they are cleared from systemic circulation. Each IgM heavy chain constant region has five sites of asparagine-(N-)linked glycosylation, and the J-chain has one N-linked glycosylation site. Thus, a pentameric, J-chain containing IgM contains up to 51 glycan moieties, which results in a complex glycosylation profile (Hennicke, J., et al., Anal. Biochem. 539:162-166 (2017)). The complexity of glycans can make manufacture of homogenously glycosylated material difficult.

Despite the advances made in the design of multimeric antibodies, there remains a need to be able to manipulate the physical, pharmacokinetic and pharmacodynamic properties of these molecules.

### SUMMARY

This disclosure provides an isolated IgM-derived binding molecule, e.g., an IgM antibody, IgM-like antibody, or other IgM-derived binding molecule, including at least one variant IgM-derived heavy chain, where the at least one variant IgM-derived heavy chain includes a variant IgM heavy chain constant region associated with a binding domain that specifically binds to a target, where at least one asparagine(N)-linked glycosylation motif of the variant IgM heavy chain constant region is mutated to prevent glycosylation at that motif, and where the N-linked glycosylation motif includes the amino acid sequence N-X₁-S/T, where N is asparagine, X₁ is any amino acid except proline, and S/T is serine or threonine. In certain embodiments, the variant IgM heavy chain constant region is derived from a human IgM heavy chain constant region that includes five N-linked glycosylation motifs N-X₁-S/T starting at amino acid positions corresponding to amino acid 46 (motif N1), amino acid 209 (motif N2), amino acid 272 (motif N3), amino acid 279 (motif N4), and amino acid 440 (motif N5) of SEQ ID NO: 1 (allele IGHM*03) or SEQ ID NO: 2 (allele IGHM*04). In certain embodiments, at least one, at least two, at least three, or at least four of the N-X₁-S/T motifs includes an amino acid insertion, deletion, or substitution that prevents glycosylation at that motif. In certain embodiments, the IgM-derived binding molecule can include an amino acid insertion, deletion, or substitution at motif N1, motif N2, motif N3, motif N5, or any combination of two or more, three or more, or all four of motifs N1, N2, N3, or N5, where the amino acid insertion, deletion, or substitution prevents glycosylation at that motif.

In certain embodiments, the IgM-derived binding molecule can include an amino acid substitution at an amino acid position corresponding to amino acid N46, N209, N272, or N440 of SEQ ID NO: 1 or SEQ ID NO: 2 where the substituted amino acid is any amino acid, an amino acid substitution at an amino acid position corresponding to amino acid S48, S211, S274, or S442 of SEQ ID NO: 1 or SEQ ID NO: 2 where the substituted amino acid is any amino acid except threonine, or any combination of two or more, three or more, or four or more of the amino acid substitutions. In certain embodiments, the amino acid substitution can correspond to N46X₂, N46A, N46D, N46Q, N46K, 548X₃, S48A, N229X₂, N229A, N229D, N229Q, N229K, S231X₃, S231A, N272X₂, N272A, N272D, N272Q, N272K, 5274X₃, S274A, N440X₂, N440A, N440D, N449Q, N449K, S242X₃, or S424A of SEQ ID NO: 1 or SEQ ID NO: 2, or any combination of two or more, three or more, or four or more of the amino acid substitutions, where X₂ is any amino acid and X₃ is any amino acid except threonine.

In certain embodiments, the variant IgM heavy chain constant region is a variant human IgM constant region that includes the amino acid sequence SEQ ID NO: 3, SEQ ID NO: 4, SEQ ID NO: 5, SEQ ID NO: 6, SEQ ID NO: 7, SEQ ID NO: 8, SEQ ID NO: 11, SEQ ID NO: 12, SEQ ID NO: 13, SEQ ID NO: 14, SEQ ID NO: 15, SEQ ID NO: 16, SEQ ID NO: 17, or SEQ ID NO: 18.

In certain embodiments, the variant IgM heavy chain constant region is mutated to introduce at least one new asparagine(N)-linked glycosylation motif into the variant IgM heavy chain constant region, where the at least one new asparagine(N)-linked glycosylation motif is introduced at a site in the variant IgM heavy chain constant region that is not naturally glycosylated in an IgM antibody. In certain embodiments, the new asparagine(N)-linked glycosylation motif is at a position in the variant IgM heavy chain constant region that corresponds to the position of an asparagine(N)-linked glycosylation motif present in a different immunoglobulin isotype, for example, a human immunoglobulin isotype selected from the group consisting of human IgG1, human IgG2, human IgG3, human IgG4, human IgA1, human IgA2, human IgD, and human IgE.

In certain embodiments, the target is a target epitope, a target antigen, a target cell, a target organ, or a target virus.

In certain embodiments, the IgM-derived binding molecule is a pentameric or a hexameric IgM antibody that includes five or six bivalent IgM binding units, respectively, where each binding unit includes two IgM heavy chains each including a VH situated amino terminal to the variant IgM constant region, and two immunoglobulin light chains each including a light chain variable domain (VL) situated amino terminal to an immunoglobulin light chain constant region, and where the VH and VL combine to form an antigen-binding domain that specifically binds to the target. In certain embodiments, the five or six IgM binding units are identical.

In certain embodiments the IgM-derived binding molecule is pentameric, and further includes a J-chain, or functional fragment thereof, or a functional variant thereof. In certain embodiments, the J-chain is a mature human J-chain that includes the amino acid sequence SEQ ID NO: 20 or a functional fragment thereof, or a functional variant thereof. In certain embodiments, the J-chain is a functional variant J-chain including one or more single amino acid substitutions, deletions, or insertions relative to a reference J-chain identical to the variant J-chain except for the one or more single amino acid substitutions, deletions, or insertions, and the IgM-derived binding molecule that includes the variant J-chain exhibits an increased serum half-life upon administration to a subject animal relative to a reference IgM-derived binding molecule that is identical except for the one or more single amino acid substitutions, deletions, or insertions in the variant J-chain, and is administered in the same way to the same animal species. In certain embodiments, the variant J-chain or functional fragment thereof includes one, two, three, or four single amino acid substitutions, deletions, or insertions relative to the reference J-chain. In certain embodiments, the variant J-chain or functional fragment thereof includes an amino acid substitution at the amino acid position corresponding to amino acid Y102 of the wild-type mature human J-chain (SEQ ID NO: 20), for example, the amino acid corresponding to Y102 of SEQ ID NO: 20 can be substituted with alanine (A). In certain embodiments the J-chain is the variant human J-chain J*, which includes the amino acid sequence SEQ ID NO: 21.

In certain embodiments, the variant J-chain or functional fragment thereof includes an a mutation within the asparagine(N)-linked glycosylation motif N-X₁-S/T starting at the amino acid position corresponding to amino acid 49 (motif N6) of the mature human J-chain (SEQ ID NO: 20), where N is asparagine, X₁ is any amino acid except proline, and S/T is serine or threonine, and where the mutation prevents glycosylation at that motif. For example, the variant J-chain or functional fragment thereof can include an amino acid substation at the amino acid position corresponding to amino acid N49 or amino acid S51 SEQ ID NO: 20 where the amino acid corresponding to S51 is not substituted with threonine (T), or where the variant J-chain includes amino acid substitutions at the amino acid positions corresponding to both amino acids N49 and S51 of SEQ ID NO: 20. In certain embodiments, the position corresponding to N49 of SEQ ID NO: 20 is substituted with alanine (A), glycine (G), threonine (T), serine (S) or aspartic acid (D). In certain embodiments the position corresponding to N49 of SEQ ID NO: 20 is substituted with alanine (A). Where the J-chain is a variant human J-chain, the J-chain includes the amino acid sequence SEQ ID NO: 22. In certain embodiments, the position corresponding to N49 of SEQ ID NO: 20 is substituted with aspartic acid (D). Where the J-chain is a variant human J-chain, the J-chain includes the amino acid sequence SEQ ID NO: 23.

In certain embodiments, the J-chain or fragment or variant thereof is a modified J-chain further including a heterologous moiety, where the heterologous moiety is fused or conjugated to the J-chain or fragment or variant thereof. In certain embodiments the heterologous moiety is a polypeptide fused to the J-chain or fragment or variant thereof. For example, the heterologous polypeptide can be fused to the J-chain or fragment or variant thereof via a peptide linker, including, e.g., at least 5 amino acids, but no more than 25 amino acids, for example, the peptide linker can consist of GGGGSGGGGSGGGGS (SEQ ID NO: 29). In certain embodiments, the heterologous polypeptide can be fused to the N-terminus of the J-chain or fragment or variant thereof, the C-terminus of the J-chain or fragment or variant thereof, or to both the N-terminus and C-terminus of the J-chain or fragment or variant thereof. In certain embodiments the heterologous polypeptide includes a binding domain, for example, an antibody or antigen-binding fragment thereof. In certain embodiments the antigen-binding fragment is a scFv fragment. In certain embodiments, the heterologous scFv fragment specifically binds to CD3ε. In certain embodiments, the modified J-chain includes the amino acid sequence SEQ ID NO: 24 (V15J), SEQ ID NO: 25 (V15J*), SEQ ID NO: 26 (V15J N49D), or SEQ ID NO: 55 (SP) or SEQ ID NOs: 20, 21, 22, or 23 fused via a peptide linker to an anti-CD3c scFv including HCDR1, HCDR2, HCDR3, LCDR1, LCDR2, and LCDR3 amino acid sequences including SEQ ID NO: 48, SEQ ID NO: 49, SEQ ID NO: 50, SEQ ID NO: 52, SEQ ID NO: 53, and SEQ ID NO: 54; SEQ ID NO: 57, SEQ ID NO: 59, SEQ ID NO: 62, SEQ ID NO: 65, SEQ ID NO: 67, and SEQ ID NO: 69; SEQ ID NO: 57, SEQ ID NO: 59, SEQ ID NO: 62, SEQ ID NO: 65, SEQ ID NO: 67, and SEQ ID NO: 70; SEQ ID NO: 58, SEQ ID NO: 60, SEQ ID NO: 63, SEQ ID NO: 66, SEQ ID NO: 68, and SEQ ID NO: 71; SEQ ID NO: 58, SEQ ID NO: 61, SEQ ID NO: 63, SEQ ID NO: 66, SEQ ID NO: 68, and SEQ ID NO: 72; SEQ ID NO: 58, SEQ ID NO: 61, SEQ ID NO: 64, SEQ ID NO: 66, SEQ ID NO: 68, and SEQ ID NO: 73, respectively.

The disclosure further provides a polynucleotide including a nucleic acid sequence that encodes the at least one variant IgM-derived heavy chain as provided herein, or a composition that includes such a polynucleotide. In certain embodiments the composition can further include a nucleic acid sequence that encodes a light chain polypeptide subunit. In certain embodiments the nucleic acid sequence encoding the at least one variant IgM-derived heavy chain and the nucleic acid sequence encoding the light chain polypeptide subunit are on separate vectors. In certain embodiments they are on a single vector. In certain embodiments the provided composition can further include a nucleic acid sequence that encodes a J-chain, or functional fragment thereof, or a functional variant thereof. In certain embodiments the nucleic acid sequence encoding the at least one variant IgM-derived heavy chain, the nucleic acid sequence encoding the light chain polypeptide subunit, and the nucleic acid sequence encoding the J-chain are on a single vector or can be on two or more separate vectors. Such vectors are provided by the disclosure. The disclosure also provides a host cell that includes any one or more of the provided polynucleotides, or vectors. The disclosure also provides a method of producing the provided IgM-derived binding molecule, where the method includes culturing the provided host cell, and recovering the constant region or antibody.

### BRIEF DESCRIPTION OF THE DRAWINGS/FIGURES

FIGS. 1A-1B show an alignment of the heavy chain constant regions of the various human immunoglobulin isotypes and subtypes, human IgG1 (IGHG1, SEQ ID NO: 34, amino acids 141-470 of GenBank AIC63046.1), human IgG2 (IGHG2, SEQ ID NO: 35, amino acids 1-326 of GenBank AXN93662.2), human IgG3 (IGHG3, SEQ ID NO: 36, amino acids 1 to 377 of GenBank AXN93659.2), human IgG4 (IGHG4, SEQ ID NO: 37, amino acids 1 to 327 of GenBank sp|P01861.1), human IgA1 (IGHA1, SEQ ID NO: 38, amino acids 144 to 496 of GenBank AIC59035.1), human IgA2 (IGHA2, SEQ ID NO: 39, amino acids 1 to 340 of GenBank P01877.4), human IgD (IGHD, SEQ ID NO: 40, amino acids 1 to 384 of GenBank P01880.3), human IgE (IGHE, SEQ ID NO: 41, amino acids 1-428 of GenBank P01854.1), and human IgM (IGHM, allele IGHM*04, SEQ ID NO: 2). Asparagine (N)-linked glycosylation motifs are shown by double-underline, with the asparagine residues in bold. Cysteine (C) amino acid residues that participate in intra-chain disulfide bonds are indicated by arrows, and cysteine residues that participate in inter-chain disulfide bonds are indicated by a barbell shape. FIG. 1A shows the CH1 domains, hinge regions or equivalent domains, and CH2/CH3 domains. FIG. 1B shows the CH3/CH4 domains and the tail-piece domains.

FIGS. 2A-2B show an alignment of the human IgM heavy chain constant region amino acid sequence (allele IGHM*04, SEQ ID NO: 2) with those of mouse (GenBank: CAC20701.1, SEQ ID NO: 42), cynomolgus monkey (amino acids 14 to 487 of GenBank: EHH62210.1, SEQ ID NO: 43), rhesus monkey (amino acids 147 to 600 of GenBank: EHH28233.1, SEQ ID NO: 45), chimpanzee (GenBank: PNI88330.1, SEQ ID NO: 44), and Sumatran orangutan (GenBank: PNJ04968.1, SEQ ID NO: 46). The amino acids corresponding to asparagine (N)-linked glycosylation motifs are boxed.

FIG. 3 is a space-filling model of a human IgM heavy chain, showing the positions of the five N-linked glycosylation sites.

FIG. 4 shows a stained, non-reducing polyacrylamide gel showing the expression and assembly of IgM+VJH modified J-chain glycovariants with single alanine mutations at N1, N2, N3, N4, N5, and N6.

FIG. 5 shows a stained, non-reducing polyacrylamide gel and a western blot (reacted with anti-J-chain antibody) showing the expression and assembly of IgM+VJH modified J-chain glycovariants with single aspartic acid mutations at N1, N2, N3, N4, N5, and N6.

FIG. 6 shows a western blot of a non-reducing polyacrylamide gel reacted with anti-J-chain antibody, showing the expression and assembly of IgM+VJH modified J-chain glycovariants with double aspartic acid mutations at N1 and N2, N2 and N3, N1 and N3, N1 and N5 and N6.

FIG. 7 shows ELISA binding of glycomutants to target antigen.

### DETAILED DESCRIPTION

### Definitions

As used herein, the term “a” or “an” entity refers to one or more of that entity; for example, “a binding molecule,” is understood to represent one or more binding molecules. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various embodiments or embodiments of the disclosure, which can be had by reference to the specification as a whole. Accordingly, the terms defined immediately below are more fully defined by reference to the specification in its entirety.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, and derivatization by known protecting/blocking groups, proteolytic cleavage, or modification by non-naturally occurring amino acids. A polypeptide can be derived from a biological source or produced by recombinant technology but is not necessarily translated from a designated nucleic acid sequence. It can be generated in any manner, including by chemical synthesis.

A polypeptide as disclosed herein can be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides which do not possess a defined three-dimensional structure, but rather can adopt many different conformations and are referred to as unfolded. As used herein, the term glycoprotein refers to a protein coupled to at least one carbohydrate moiety that is attached to the protein via an oxygen-containing or a nitrogen-containing side chain of an amino acid, e.g., a serine or an asparagine.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are native or recombinant polypeptides which have been separated, fractionated, or partially or substantially purified by any suitable technique.

As used herein, the term “a non-naturally occurring polypeptide” or any grammatical variants thereof, is a conditional definition that explicitly excludes, but only excludes, those forms of the polypeptide that are, or might be, determined or interpreted by a judge or an administrative or judicial body, to be “naturally-occurring.”

Other polypeptides disclosed herein are fragments, derivatives, analogs, or variants of the foregoing polypeptides, and any combination thereof. The terms “fragment,” “variant,” “derivative” and “analog” as disclosed herein include any polypeptides which retain at least some of the properties of the corresponding native antibody or polypeptide, for example, specifically binding to an antigen. Fragments of polypeptides include, for example, proteolytic fragments, as well as deletion fragments, in addition to specific antibody fragments discussed elsewhere herein. Variants of, e.g., a polypeptide include fragments as described above, and also polypeptides with altered amino acid sequences due to amino acid substitutions, deletions, or insertions. In certain embodiments, variants can be non-naturally occurring. Non-naturally occurring variants can be produced using art-known mutagenesis techniques. Variant polypeptides can comprise conservative or non-conservative amino acid substitutions, deletions or additions. Derivatives are polypeptides that have been altered so as to exhibit additional features not found on the original polypeptide. Examples include fusion proteins. Variant polypeptides can also be referred to herein as “polypeptide analogs.” As used herein a “derivative” of a polypeptide can also refer to a subject polypeptide having one or more amino acids chemically derivatized by reaction of a functional side group. Also included as “derivatives” are those peptides that contain one or more derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the polypeptides, binding molecules, and antibodies of the present disclosure do not abrogate the binding of the polypeptide, binding molecule, or antibody containing the amino acid sequence, to the antigen to which the antibody binds. Methods of identifying nucleotide and amino acid conservative substitutions which do not eliminate antigen-binding are well-known in the art (see, e.g., Brummell et al., Biochem. 32: 1180-1 187 (1993); Kobayashi et al., Protein Eng. 12(10):879-884 (1999); and Burks et al., Proc. Natl. Acad. Sci. USA 94: 412-417 (1997)).

The term “polynucleotide” is intended to encompass a singular nucleic acid as well as plural nucleic acids and refers to an isolated nucleic acid molecule or construct, e.g., messenger RNA (mRNA), cDNA, or plasmid DNA (pDNA). A polynucleotide can comprise a conventional phosphodiester bond or a non-conventional bond (e.g., an amide bond, such as found in peptide nucleic acids (PNA)). The terms “nucleic acid” or “nucleic acid sequence” refer to any one or more nucleic acid segments, e.g., DNA or RNA fragments, present in a polynucleotide.

By an “isolated” nucleic acid or polynucleotide is intended any form of the nucleic acid or polynucleotide that is separated from its native environment. For example, gel-purified polynucleotide, or a recombinant polynucleotide encoding a polypeptide contained in a vector would be considered to be “isolated.” Also, a polynucleotide segment, e.g., a PCR product, which has been engineered to have restriction sites for cloning is considered to be “isolated.” Further examples of an isolated polynucleotide include recombinant polynucleotides maintained in heterologous host cells or purified (partially or substantially) polynucleotides in a non-native solution such as a buffer or saline. Isolated RNA molecules include in vivo or in vitro RNA transcripts of polynucleotides, where the transcript is not one that would be found in nature. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, the term “a non-naturally occurring polynucleotide” or any grammatical variants thereof, is a conditional definition that explicitly excludes, but only excludes, those forms of the nucleic acid or polynucleotide that are, or might be, determined or interpreted by a judge, or an administrative or judicial body, to be “naturally-occurring.”

As used herein, a “coding region” is a portion of nucleic acid which consists of codons translated into amino acids. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode an immunoglobulin heavy chain variable region and an immunoglobulin light chain variable region. In addition, a vector, polynucleotide, or nucleic acid can include heterologous coding regions, either fused or unfused to another coding region. Heterologous coding regions include without limitation, those encoding specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally can include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA in predetermined cells. Other transcription control elements, besides a promoter, for example enhancers, operators, repressors, and transcription termination signals, can be operably associated with the polynucleotide to direct cell-specific transcription.

A variety of transcription control regions are known to those skilled in the art. These include, without limitation, transcription control regions that function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide can be RNA, for example, in the form of messenger RNA (mRNA), transfer RNA, or ribosomal RNA.

Polynucleotide and nucleic acid coding regions can be associated with additional coding regions which encode secretory or signal peptides, which direct the secretion of a polypeptide encoded by a polynucleotide as disclosed herein. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells can have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse β-glucuronidase.

As used herein, the term “binding molecule” refers in its broadest sense to a molecule that specifically binds to a receptor, e.g., an epitope or an antigenic determinant. As described further herein, a binding molecule can comprise one of more “binding domains,” e.g., “antigen-binding domains” described herein. A non-limiting example of a binding molecule is an antibody or antibody-like molecule as described in detail herein that retains antigen-specific binding. In certain embodiments a “binding molecule” comprises an antibody or antibody-like or antibody-derived molecule as described in detail herein.

As used herein, the terms “binding domain” or “antigen-binding domain” (can be used interchangeably) refer to a region of a binding molecule, e.g., an antibody or antibody-like, or antibody-derived molecule, that is necessary and sufficient to specifically bind to a target, e.g., an epitope, a polypeptide, a cell, or an organ. For example, an “Fv,” e.g., a heavy chain variable region and a light chain variable region of an antibody, either as two separate polypeptide subunits or as a single chain, is considered to be a “binding domain.” Other antigen-binding domains include, without limitation, a single domain heavy chain variable region (VHH) of an antibody derived from a camelid species, or six immunoglobulin complementarity determining regions (CDRs) expressed in a fibronectin scaffold. A “binding molecule,” or “antibody” as described herein can include one, two, three, four, five, six, seven, eight, nine, ten, eleven, twelve or more “antigen-binding domains.”

The terms “antibody” and “immunoglobulin” can be used interchangeably herein. An antibody (or a fragment, variant, or derivative thereof as disclosed herein, e.g., an IgM-like antibody) includes at least the variable domain of a heavy chain (e.g., from a camelid species) or at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988). Unless otherwise stated, the term “antibody” encompasses anything ranging from a small antigen-binding fragment of an antibody to a full sized antibody, e.g., an IgG antibody that includes two complete heavy chains and two complete light chains, an IgA antibody that includes four complete heavy chains and four complete light chains and includes a J-chain and/or a secretory component, or an IgM-derived binding molecule, e.g., an IgM antibody or IgM-like antibody, that includes ten or twelve complete heavy chains and ten or twelve complete light chains and optionally includes a J-chain or functional fragment or variant thereof.

The term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4 or α1-α2)). It is the nature of this chain that determines the “isotype” of the antibody as IgG, IgM, IgA IgD, or IgE, respectively. The immunoglobulin subclasses (subtypes) e.g., IgG₁, IgG₂, IgG₃, IgG₄, IgA₁, IgA₂, etc. are well characterized and are known to confer functional specialization. Modified versions of each of these immunoglobulins are readily discernible to the skilled artisan in view of the instant disclosure and, accordingly, are within the scope of this disclosure.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are expressed, e.g., by hybridomas, B cells or genetically engineered host cells. In the heavy chain, the amino acid sequences run from an N-terminus at the forked ends of the Y configuration to the C-terminus at the bottom of each chain. The basic structure of certain antibodies, e.g., IgG antibodies, includes two heavy chain subunits and two light chain subunits covalently connected via disulfide bonds to form a “Y” structure, also referred to herein as an “H2L2” structure, or a “binding unit.”

The term “binding unit” is used herein to refer to the portion of a binding molecule, e.g., an antibody, antibody-like molecule, or antibody-derived molecule, antigen-binding fragment thereof, or multimerizing fragment thereof, which corresponds to a standard “H2L2” immunoglobulin structure, i.e., two heavy chains or fragments thereof and two light chains or fragments thereof. In certain embodiments, e.g., where the binding molecule is a bivalent IgG antibody or antigen-binding fragment thereof, the terms “binding molecule” and “binding unit” are equivalent. In other embodiments, e.g., where the binding molecule is multimeric, e.g., a dimeric IgA antibody or IgA-like antibody, a pentameric IgM antibody or IgM-like antibody, or a hexameric IgM antibody or IgM-like antibody, or any derivative thereof, the binding molecule comprises two or more “binding units.” Two in the case of an IgA dimer, or five or six in the case of an IgM pentamer or hexamer, respectively. A binding unit need not include full-length antibody heavy and light chains, but will typically be bivalent, i.e., will include two “antigen-binding domains,” as defined above. As used herein, certain binding molecules provided in this disclosure are “dimeric,” and include two bivalent binding units that include IgA constant regions or multimerizing fragments thereof. Certain binding molecules provided in this disclosure are “pentameric” or “hexameric,” and include five or six bivalent binding units that include IgM constant regions or multimerizing fragments or variants thereof. A binding molecule, e.g., an antibody or antibody-like molecule or antibody-derived binding molecule, comprising two or more, e.g., two, five, or six binding units, is referred to herein as “multimeric.”

The term “J-chain” as used herein refers to the J-chain of native sequence IgM or IgA antibodies of any animal species, any functional fragment thereof, derivative thereof, and/or variant thereof, including a mature human J-chain, the amino acid sequence of which is presented as SEQ ID NO: 20. Various J-chain variants and modified J-chain derivatives are disclosed herein. As persons of ordinary skill in the art will recognize, “a functional fragment” or a “functional variant” includes those fragments and variants that can associate with IgM heavy chain constant regions to form a pentameric IgM antibody (or alternatively can associate with IgA heavy chain constant regions to form a dimeric IgA antibody).

The term “modified J-chain” is used herein to refer to a derivative of a native sequence J-chain polypeptide comprising a heterologous moiety, e.g., a heterologous polypeptide, e.g., an extraneous binding domain or functional domain introduced into or attached to the native J-chain sequence. The introduction can be achieved by any means, including direct or indirect fusion of the heterologous polypeptide or other moiety or by attachment through a peptide or chemical linker. The term “modified human J-chain” encompasses, without limitation, a native sequence human J-chain of the amino acid sequence of SEQ ID NO: 20 or functional fragment thereof, or functional variant thereof, modified by the introduction of a heterologous moiety, e.g., a heterologous polypeptide, e.g., an extraneous binding domain. In certain embodiments the heterologous moiety does not interfere with efficient polymerization of IgM into a pentamer or IgA into a dimer, and binding of such polymers to a target. Exemplary modified J-chains can be found, e.g., in U.S. Pat. Nos. 9,951,134 and 10,618,978 and in U.S. Patent Application Publication No. US-2019-0185570, each of which is incorporated herein by reference in its entirety.

As used herein the term “IgM-derived binding molecule” refers collectively to native IgM antibodies, IgM-like antibodies, as well as other IgM-derived binding molecules comprising non-antibody binding and/or functional domains instead of an antibody antigen binding domain or subunit thereof, and any fragments, e.g., multimerizing fragments, variants, or derivatives thereof.

As used herein, the term “IgM-like antibody” refers generally to a variant antibody or antibody-derived binding molecule that still retains the ability to form hexamers, or in association with J-chain, form pentamers. An IgM-like antibody or other IgM-derived binding molecule typically includes at least the Cμ4-tp domains of the IgM constant region but can include heavy chain constant region domains from other antibody isotypes, e.g., IgG, from the same species or from a different species. An IgM-like antibody or other IgM-derived binding molecule can likewise be an antibody fragment in which one or more constant regions are deleted, as long as the IgM-like antibody is capable of forming hexamers and/or pentamers. Thus, an IgM-like antibody or other IgM-derived binding molecule can be, e.g., a hybrid IgM/IgG antibody or can be a “multimerizing fragment” of an IgM antibody.

The terms “valency,” “bivalent,” “multivalent” and grammatical equivalents, refer to the number of binding domains, e.g., antigen-binding domains in given binding molecule, e.g., antibody, antibody-derived, or antibody-like molecule, or in a given binding unit. As such, the terms “bivalent”, “tetravalent”, and “hexavalent” in reference to a given binding molecule, e.g., an IgM antibody, IgM-like antibody, other IgM-derived binding molecule, or multimerizing fragment thereof, denote the presence of two antigen-binding domains, four antigen-binding domains, and six antigen-binding domains, respectively. A typical IgM antibody, IgM-like antibody, or other IgM-derived binding molecule, where each binding unit is bivalent, can have 10 or 12 valencies. A bivalent or multivalent binding molecule, e.g., antibody or antibody-derived molecule, can be monospecific, i.e., all of the antigen-binding domains are the same, or can be bispecific or multispecific, e.g., where two or more antigen-binding domains are different, e.g., bind to different epitopes on the same antigen, or bind to entirely different antigens.

The term “epitope” includes any molecular determinant capable of specific binding to an antigen-binding domain of an antibody, antibody-like, or antibody-derived molecule. In certain embodiments, an epitope can include chemically active surface groupings of molecules such as amino acids, sugar side chains, phosphoryl groups, or sulfonyl groups, and, in certain embodiments, can have three-dimensional structural characteristics, and or specific charge characteristics. An epitope is a region of a target that is bound by an antigen-binding domain of an antibody.

The term “target” is used in the broadest sense to include substances that can be bound by a binding molecule, e.g., antibody, antibody-like, or antibody-derived molecule. A target can be, e.g., a polypeptide, a nucleic acid, a carbohydrate, a lipid, or other molecule, or a minimal epitope on such molecule. Moreover, a “target” can, for example, be a cell, an organ, or an organism, e.g., an animal, plant, microbe, or virus, that comprises an epitope that can be bound by a binding molecule, e.g., antibody, antibody-like, or antibody-derived molecule.

Both the light and heavy chains of antibodies, antibody-like, or antibody-derived molecules are divided into regions of structural and functional homology. The terms “constant” and “variable” are used functionally. In this regard, it will be appreciated that the variable domains of both the variable light (VL) and variable heavy (VH) chain portions determine antigen recognition and specificity. Conversely, the constant region domains of the light chain (CL) and the heavy chain (e.g., CH1, CH2, CH3, or CH4) confer biological properties such as secretion, transplacental mobility, Fc receptor binding, complement binding, and the like. By convention the numbering of the constant region domains increases as they become more distal from the antigen-binding site or amino-terminus of the antibody. The N-terminal portion is a variable region and at the C-terminal portion is a constant region; the CH3 (or CH4, e.g., in the case of IgM) and CL domains actually comprise the carboxy-terminus of the heavy and light chain, respectively.

A “full length IgM antibody heavy chain” is a polypeptide that includes, in N-terminal to C-terminal direction, an antibody heavy chain variable domain (VH), an antibody heavy chain constant domain 1 (CM1 or Cμ1), an antibody heavy chain constant domain 2 (CM2 or Cμ2), an antibody heavy chain constant domain 3 (CM3 or Cμ3), and an antibody heavy chain constant domain 4 (CM4 or Cμ4) that can include a tailpiece.

As indicated above, variable region(s) allow a binding molecule, e.g., antibody, antibody-like, or antibody-derived molecule, to selectively recognize and specifically bind epitopes on antigens. That is, the VL domain and VH domain, or subset of the complementarity determining regions (CDRs), of a binding molecule, e.g., an antibody, antibody-like, or antibody-derived molecule, combine to form the antigen-binding domain. More specifically, an antigen-binding domain can be defined by three CDRs on each of the VH and VL chains. Certain antibodies form larger structures. For example, IgA can form a molecule that includes two H2L2 binding units and a J-chain covalently connected via disulfide bonds, which can be further associated with a secretory component, and IgM can form a pentameric or hexameric molecule that includes five or six H2L2 binding units and optionally a J-chain covalently connected via disulfide bonds.

The six “complementarity determining regions” or “CDRs” present in an antibody antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domain, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids that make up the CDRs and the framework regions, respectively, can be readily identified for any given heavy or light chain variable region by one of ordinary skill in the art, since they have been defined in various different ways (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. These particular regions have been described, for example, by Kabat et al., U.S. Dept. of Health and Human Services, “Sequences of Proteins of Immunological Interest” (1983) and by Chothia et al., J. Mol. Biol. 196:901-917 (1987), which are incorporated herein by reference. The Kabat and Chothia definitions include overlapping or subsets of amino acids when compared against each other. Nevertheless, application of either definition (or other definitions known to those of ordinary skill in the art) to refer to a CDR of an antibody or variant thereof is intended to be within the scope of the term as defined and used herein, unless otherwise indicated. The appropriate amino acids which encompass the CDRs as defined by each of the above cited references are set forth below in Table 1 as a comparison. The exact amino acid numbers which encompass a particular CDR will vary depending on the sequence and size of the CDR. Those skilled in the art can routinely determine which amino acids comprise a particular CDR given the variable region amino acid sequence of the antibody.

TABLE 1
CDR Definitions*

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

*Numbering of all CDR definitions in Table 1 is according to the numbering conventions set forth by Kabat et al. (see below).

Antibody variable domains can also be analyzed, e.g., using the IMGT information system (imgt_dot_cines_dot_fr/) (IMGT®/V-Quest) to identify variable region segments, including CDRs. (See, e.g., Brochet et al., Nucl. Acids Res. 36:W503-508, 2008).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless use of the Kabat numbering system is explicitly noted, however, consecutive numbering is used for all amino acid sequences in this disclosure.

The Kabat numbering system for the human IgM constant domain can be found in Kabat, et. al. “Tabulation and Analysis of Amino acid and nucleic acid Sequences of Precursors, V-Regions, C-Regions, J-Chain, T-Cell Receptors for Antigen, T-Cell Surface Antigens, β-2 Microglobulins, Major Histocompatibility Antigens, Thy-1, Complement, C-Reactive Protein, Thymopoietin, Integrins, Post-gamma Globulin, α-2 Macroglobulins, and Other Related Proteins,” U.S. Dept. of Health and Human Services (1991). IgM constant regions can be numbered sequentially (i.e., amino acid #1 starting with the first amino acid of the constant region, or by using the Kabat numbering scheme. A comparison of the numbering of two alleles of the human IgM constant region sequentially (presented herein as SEQ ID NO: 1 (allele IGHM*03) and SEQ ID NO: 2 (allele IGHM*04)) and by the Kabat system is set out below. The underlined amino acid residues are not accounted for in the Kabat system (“X,” double underlined below, can be serine (S) (SEQ ID NO: 1) or glycine (G) (SEQ ID NO: 2)):

```
Sequential (SEQ ID NO: 1 or SEQ ID NO: 2)/
KABAT numbering key for IgM heavy chain
    1/127 GSASAPTLFP LVSCENSPSD TSSVAVGCLA QDFLPDSITF
                SWKYKNNSDI
  51/176 SSTRGFPSVL RGGKYAATSQ VLLPSKDVMQ GTDEHVVCKV
                QHPNGNKEKN
101/226 VPLPVIAELP PKVSVFVPPR DGFFGNPRKS KLICQATGFS
                PRQIQVSWLR
151/274 EGKQVGSGVT TDQVQAEAKE SGPTTYKVTS TLTIKESDWL
                XQSMFTCRVD
201/324 HRGLTFQQKA SSMCVPDQDT AIRVFAIPPS FASIFLTKST
                KLTCLVTDLT
251/374 TYDSVTISWT RQNGEAVKTH TNISESHPNA TFSAVGEASI
                CEDDWNSGER
301/424 FTCTVTHTDL PSPLKQTISR PKGVALHRPD VYLLPPAREQ
                LNLRESATIT
351/474 CLVTGFSPAD VFVQWMQRGQ PLSPEKYVTS APMPEPQAPG
                RYFAHSILTV
401/524 SEEEWNTGET YTCVVAHEAL PNRVTERTVD KSTGKPTLYN
                VSLVMSDTAG
451/574 TCY
```

Binding molecules, e.g., antibodies, antibody-like, or antibody-derived molecules, antigen-binding fragments, variants, or derivatives thereof, and/or multimerizing fragments thereof include, but are not limited to, polyclonal, monoclonal, human, humanized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019.

By “specifically binds,” it is generally meant that a binding molecule, e.g., an antibody or fragment, variant, or derivative thereof binds to an epitope via its antigen-binding domain, and that the binding entails some complementarity between the antigen-binding domain and the epitope. According to this definition, a binding molecule, e.g., antibody, antibody-like, or antibody-derived molecule, is said to “specifically bind” to an epitope when it binds to that epitope, via its antigen-binding domain more readily than it would bind to a random, unrelated epitope. The term “specificity” is used herein to qualify the relative affinity by which a certain binding molecule binds to a certain epitope. For example, binding molecule “A” can be deemed to have a higher specificity for a given epitope than binding molecule “B,” or binding molecule “A” can be said to bind to epitope “C” with a higher specificity than it has for related epitope “D.”

A binding molecule, e.g., an antibody or fragment, variant, or derivative thereof disclosed herein can be said to bind a target antigen with an off rate (k(off)) of less than or equal to 5×10⁻² sec⁻¹, 10⁻² sec⁻¹, 5×10⁻³ sec⁻¹, 10⁻³ sec⁻¹, 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10⁻⁷ sec⁻¹, 5×10⁻⁷ sec⁻¹ or 10⁻⁷ sec⁻¹.

A binding molecule, e.g., an antibody or antigen-binding fragment, variant, or derivative disclosed herein can be said to bind a target antigen with an on rate (k(on)) of greater than or equal to 10³ M⁻¹ sec⁻¹, 5×10³ M⁻¹ sec⁻¹, 10⁴ M⁻¹ sec⁻¹, 5×10⁴ M⁻¹ sec⁻¹, 10⁵ M⁻¹ sec⁻¹, 5×10⁵ M⁻¹ sec⁻¹, 10⁶M⁻¹ sec⁻¹, or 5×10⁶ M⁻¹ sec⁻¹ or 10⁷ M⁻¹ sec⁻¹.

A binding molecule, e.g., an antibody or fragment, variant, or derivative thereof is said to competitively inhibit binding of a reference antibody or antigen-binding fragment to a given epitope if it preferentially binds to that epitope to the extent that it blocks, to some degree, binding of the reference antibody or antigen-binding fragment to the epitope. Competitive inhibition can be determined by any method known in the art, for example, competition ELISA assays. A binding molecule can be said to competitively inhibit binding of the reference antibody or antigen-binding fragment to a given epitope by at least 90%, at least 80%, at least 70%, at least 60%, or at least 50%.

As used herein, the term “affinity” refers to a measure of the strength of the binding of an individual epitope with one or more antigen-binding domains, e.g., of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of antigen-binding domains and an antigen. See, e.g., Harlow at pages 29-34. Avidity is related to both the affinity of individual antigen-binding domains in the population with specific epitopes, and also the valencies of the immunoglobulins and the antigen. For example, the interaction between a bivalent monoclonal antibody and an antigen with a highly repeating epitope structure, such as a polymer, would be one of high avidity. An interaction between a bivalent monoclonal antibody with a receptor present at a high density on a cell surface would also be of high avidity.

Binding molecules, e.g., antibodies or fragments, variants, or derivatives thereof as disclosed herein can also be described or specified in terms of their cross-reactivity. As used herein, the term “cross-reactivity” refers to the ability of a binding molecule, e.g., an antibody or fragment, variant, or derivative thereof, specific for one antigen, to react with a second antigen; a measure of relatedness between two different antigenic substances. Thus, a binding molecule is cross reactive if it binds to an epitope other than the one that induced its formation. The cross-reactive epitope generally contains many of the same complementary structural features as the inducing epitope, and in some cases, can actually fit better than the original.

A binding molecule, e.g., an antibody or fragment, variant, or derivative thereof can also be described or specified in terms of their binding affinity to an antigen. For example, a binding molecule can bind to an antigen with a dissociation constant or K_{D} no greater than 5×10⁻² M, 10⁻² M, 5×10⁻³M, 10⁻³M, 5×10⁻⁴ M, 10⁻⁴ M, 5×10⁻⁵M, 10⁻⁵M, 5×10⁻⁶ M, 10⁻⁶ M, 5×10⁻⁷ M, 10⁻⁷ M, 5×10⁻⁸M, 10⁻⁸ M, 5×10⁻⁹ M, 10⁻⁹ M, 5×10⁻¹⁰ M, 10⁻¹⁰ M 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴M, 5×10⁻¹⁵ M, or 10⁻¹⁵M.

“Antigen-binding antibody fragments” including single-chain antibodies or other antigen-binding domains can exist alone or in combination with one or more of the following: hinge region, CH1, CH2, CH3, or CH4 domains, J-chain, or secretory component. Also included are antigen-binding fragments that can include any combination of variable region(s) with one or more of a hinge region, CH1, CH2, CH3, or CH4 domains, a J-chain, or a secretory component. Binding molecules, e.g., antibodies, or antigen-binding fragments thereof can be from any animal origin including birds and mammals. The antibodies can be, e.g., human, murine, donkey, rabbit, goat, guinea pig, camel, llama, horse, or chicken antibodies. In another embodiment, the variable region can be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and can in some instances express endogenous immunoglobulins and some not, as described infra and, for example in, U.S. Pat. No. 5,939,598 by Kucherlapati et al. According to embodiments of the present disclosure, an IgM antibody, IgM-like antibody, or other IgM-derived binding molecule as provided herein can include an antigen-binding fragment of an antibody, e.g., a scFv fragment, so long as the IgM antibody, IgM-like antibody, or other IgM-derived binding molecule is able to form a multimer, e.g., a hexamer or a pentamer. As used herein such a fragment comprises a “multimerizing fragment.”

As used herein, the term “heavy chain subunit” includes amino acid sequences derived from an immunoglobulin heavy chain, a binding molecule, e.g., an antibody, antibody-like, or antibody-derived molecule comprising a heavy chain subunit can include at least one of: a VH domain, a CH1 domain, a hinge (e.g., upper, middle, and/or lower hinge region) domain, a CH2 domain, a CH3 domain, a CH4 domain, or a variant or fragment thereof. For example, a binding molecule, e.g., an antibody, antibody-like, or antibody-derived molecule, or fragment, e.g., multimerizing fragment, variant, or derivative thereof can include without limitation, in addition to a VH domain: a CH1 domain; a CH1 domain, a hinge, and a CH2 domain; a CH1 domain and a CH3 domain; a CH1 domain, a hinge, and a CH3 domain; or a CH1 domain, a hinge domain, a CH2 domain, and a CH3 domain. In certain embodiments a binding molecule, e.g., an antibody, antibody-like, or antibody-derived molecule, or fragment, e.g., multimerizing fragment, variant, or derivative thereof can include, in addition to a VH domain, a CH3 domain and a CH4 domain; or a CH3 domain, a CH4 domain, and a J-chain. Further, a binding molecule, e.g., an antibody, antibody-like, or antibody-derived molecule, for use in the disclosure can lack certain constant region portions, e.g., all or part of a CH2 domain. It will be understood by one of ordinary skill in the art that these domains (e.g., the heavy chain subunit) can be modified such that they vary in amino acid sequence from the original immunoglobulin molecule. According to embodiments of the present disclosure, an IgM antibody, IgM-like antibody, or other IgM-derived binding molecule as provided herein comprises sufficient portions of an IgM heavy chain constant region to allow the IgM antibody, IgM-like antibody, or other IgM-derived binding molecule to form a multimer, e.g., a hexamer or a pentamer. As used herein such a fragment comprises a “multimerizing fragment.”

As used herein, the term “light chain subunit” includes amino acid sequences derived from an immunoglobulin light chain. The light chain subunit includes at least a VL, and can further include a CL (e.g., Cκ or Cλ) domain.

Binding molecules, e.g., antibodies, antibody-like molecules, antibody-derived molecules, antigen-binding fragments, variants, or derivatives thereof, or multimerizing fragments thereof can be described or specified in terms of the epitope(s) or portion(s) of a target, e.g., a target antigen that they recognize or specifically bind. The portion of a target antigen that specifically interacts with the antigen-binding domain of an antibody is an “epitope,” or an “antigenic determinant.” A target antigen can comprise a single epitope or at least two epitopes, and can include any number of epitopes, depending on the size, conformation, and type of antigen.

As previously indicated, the subunit structures and three-dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of a typical IgG heavy chain molecule.

As used herein the term “disulfide bond” includes the covalent bond formed between two sulfur atoms, e.g., in cysteine residues of a polypeptide. The amino acid cysteine comprises a thiol group that can form a disulfide bond or bridge with a second thiol group. Disulfide bonds can be “intra-chain,” i.e., linking to cysteine residues in a single polypeptide or polypeptide subunit, or can be “inter-chain,” i.e., linking two separate polypeptide subunits, e.g., an antibody heavy chain and an antibody light chain, to antibody heavy chains, or an IgM or IgA antibody heavy chain constant region and a J-chain.

As used herein, the term “chimeric antibody” refers to an antibody in which the immunoreactive region or site is obtained or derived from a first species and the constant region (which can be intact, partial or modified) is obtained from a second species. In some embodiments the target binding region or site will be from a non-human source (e.g. mouse or primate) and the constant region is human.

The terms “multispecific antibody” or “bispecific antibody” refer to an antibody, antibody-like, or antibody-derived molecule that has antigen-binding domains for two or more different epitopes within a single antibody molecule. Other binding molecules in addition to the canonical antibody structure can be constructed with two binding specificities. Epitope binding by bispecific or multispecific antibodies can be simultaneous or sequential. Triomas and hybrid hybridomas are two examples of cell lines that can secrete bispecific antibodies. Bispecific antibodies can also be constructed by recombinant means. (Ströhlein and Heiss, Future Oncol. 6:1387-94 (2010); Mabry and Snavely, IDrugs. 13:543-9 (2010)). A bispecific antibody can also be a diabody.

As used herein, the term “engineered antibody” refers to an antibody in which a variable domain, constant region, and/or J-chain is altered by at least partial replacement of one or more amino acids. In certain embodiments entire CDRs from an antibody of known specificity can be grafted into the framework regions of a heterologous antibody. Although alternate CDRs can be derived from an antibody of the same class or even subclass as the antibody from which the framework regions are derived, CDRs can also be derived from an antibody of different class, e.g., from an antibody from a different species. An engineered antibody in which one or more “donor” CDRs from a non-human antibody of known specificity are grafted into a human heavy or light chain framework region is referred to herein as a “humanized antibody.” In certain embodiments not all of the CDRs are replaced with the complete CDRs from the donor variable region and yet the antigen-binding capacity of the donor can still be transferred to the recipient variable domains. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques).

As used herein, the terms “linked,” “fused” or “fusion” or other grammatical equivalents can be used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments can be physically or spatially separated by, for example, in-frame linker sequence. For example, polynucleotides encoding the CDRs of an immunoglobulin variable region can be fused, in-frame, but be separated by a polynucleotide encoding at least one immunoglobulin framework region or additional CDR regions, as long as the “fused” CDRs are co-translated as part of a continuous polypeptide.

In the context of polypeptides, a “linear sequence” or a “sequence” is an order of amino acids in a polypeptide in an amino to carboxyl terminal direction in which amino acids that neighbor each other in the sequence are contiguous in the primary structure of the polypeptide. A portion of a polypeptide that is “amino-terminal” or “N-terminal” to another portion of a polypeptide is that portion that comes earlier in the sequential polypeptide chain. Similarly, a portion of a polypeptide that is “carboxy-terminal” or “C-terminal” to another portion of a polypeptide is that portion that comes later in the sequential polypeptide chain. For example, in a typical antibody, the variable domain is “N-terminal” to the constant region, and the constant region is “C-terminal” to the variable domain.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into RNA, e.g., messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

Terms such as “treating” or “treatment” or “to treat” or “alleviating” or “to alleviate” refer to therapeutic measures that cure, slow down, lessen symptoms of, and/or halt or slow the progression of an existing diagnosed pathologic condition or disorder. Terms such as “prevent,” “prevention,” “avoid,” “deterrence” and the like refer to prophylactic or preventative measures that prevent the development of an undiagnosed targeted pathologic condition or disorder. Thus, “those in need of treatment” can include those already with the disorder; those prone to have the disorder; and those in whom the disorder is to be prevented.

As used herein the terms “serum half-life” or “plasma half-life” refer to the time it takes (e.g., in minutes, hours, or days) following administration for the serum or plasma concentration of a drug, e.g., a binding molecule such as an antibody, antibody-like, or antibody-derived molecule or fragment, e.g., multimerizing fragment thereof as described herein, to be reduced by 50%. Two half-lives can be described: the alpha half-life, a half-life, or t_{1/2}α, which is the rate of decline in plasma concentrations due to the process of drug redistribution from the central compartment, e.g., the blood in the case of intravenous delivery, to a peripheral compartment (e.g., a tissue or organ), and the beta half-life, β half-life, or t_{1/2}β which is the rate of decline due to the processes of excretion or metabolism.

As used herein the term “area under the plasma drug concentration-time curve” or “AUC” reflects the actual body exposure to drug after administration of a dose of the drug and is expressed in mg*h/L. This area under the curve is measured from time 0 (t₀) to infinity (∞) and is dependent on the rate of elimination of the drug from the body and the dose administered.

As used herein, the term “mean residence time” or “MRT” refers to the average length of time the drug remains in the body.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, swine, cows, bears, and so on.

As used herein, phrases such as “a subject that would benefit from therapy” and “an animal in need of treatment” refers to a subset of subjects, from amongst all prospective subjects, which would benefit from administration of a given therapeutic agent, e.g., a binding molecule such as an antibody, comprising one or more antigen-binding domains. Such binding molecules, e.g., antibodies, can be used, e.g., for a diagnostic procedure and/or for treatment or prevention of a disease.

IgM Antibodies, IgM-Like Antibodies, or Other IgM-Derived Binding Molecules

IgM is the first immunoglobulin produced by B cells in response to stimulation by antigen and is naturally present at around 1.5 mg/ml in serum with a half-life of about 5 days. IgM is a pentameric or hexameric molecule and thus includes five or six binding units. An IgM binding unit typically includes two light and two heavy chains. While an IgG heavy chain constant region contains three heavy chain constant domains (CH1, CH2 and CH3), the heavy (μ) constant region of IgM additionally contains a fourth constant domain (CH4) and includes a C-terminal “tailpiece.” The human IgM constant region typically comprises the amino acid sequence SEQ ID NO: 1 (identical to, e.g., GenBank Accession Nos. pir∥S37768, CAA47708.1, and. CAA47714.1, allele IGHM*03) or SEQ ID NO: 2 (identical to, e.g., GenBank Accession No. sp|P01871.4, allele IGHM*04). The human Cμ1 region ranges from about amino acid 5 to about amino acid 102 of SEQ ID NO: 1 or SEQ ID NO: 2; the human Cμ2 region ranges from about amino acid 114 to about amino acid 205 of SEQ ID NO: 1 or SEQ ID NO: 2, the human Cμ3 region ranges from about amino acid 224 to about amino acid 319 of SEQ ID NO: 1 or SEQ ID NO: 2, the Cμ4 region ranges from about amino acid 329 to about amino acid 430 of SEQ ID NO: 1 or SEQ ID NO: 2, and the tailpiece ranges from about amino acid 431 to about amino acid 453 of SEQ ID NO: 1 or SEQ ID NO: 2.

Other forms and alleles of the human IgM constant region with minor sequence variations exist, including, without limitation, GenBank Accession Nos. CAB37838.1, and pir∥MHHU. The amino acid substitutions, insertions, and/or deletions at positions corresponding to SEQ ID NO: 1 or SEQ ID NO: 2 described and claimed elsewhere in this disclosure can likewise be incorporated into alternate human IgM sequences, as well as into IgM constant region amino acid sequences of other species.

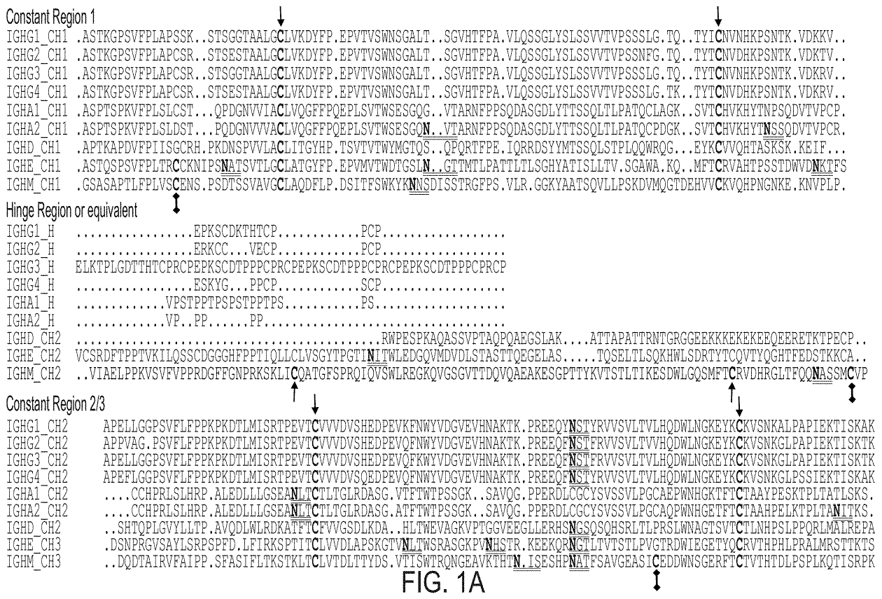

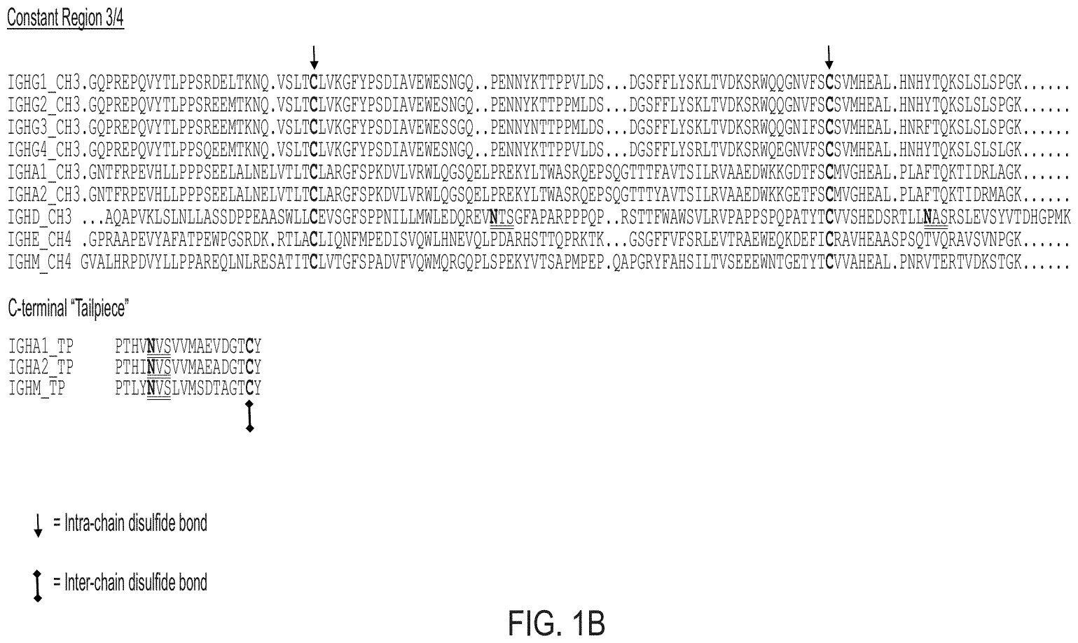

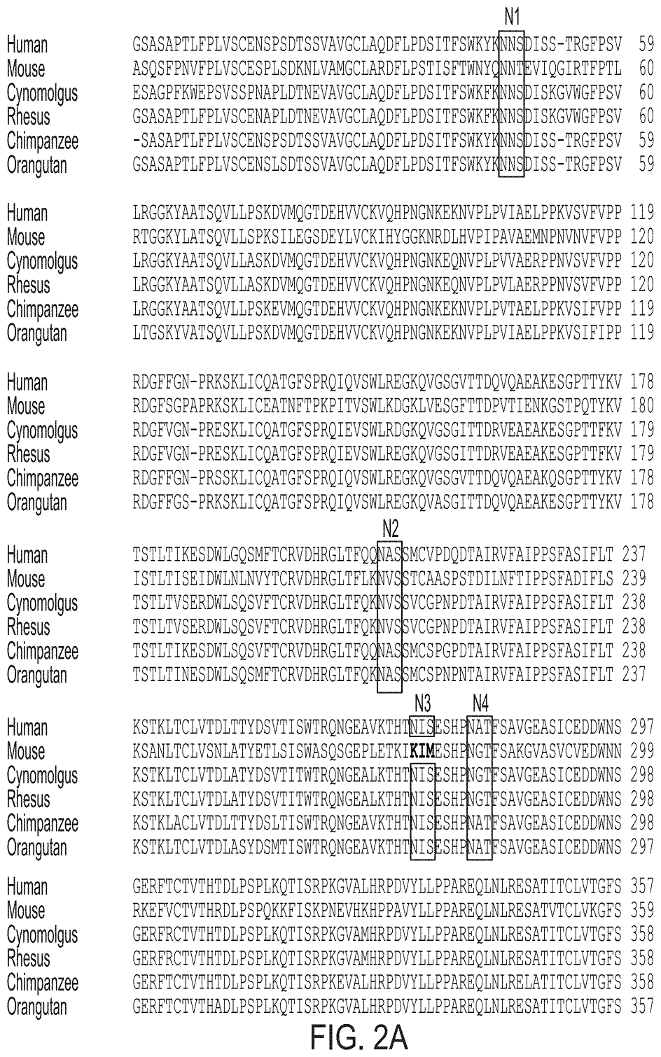

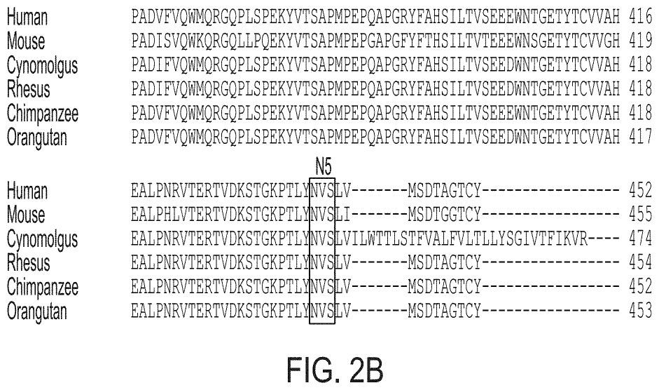

Human IgM constant regions, and also certain non-human primate IgM constant regions, as provided herein typically include five (5) naturally-occurring asparagine (N)-linked glycosylation motifs or sites. See FIG. 1. As used herein “an N-linked glycosylation motif” comprises or consists of the amino acid sequence N-X₁-S/T, wherein N is asparagine, X₁ is any amino acid except proline (P), and S/T is serine (S) or threonine (T). The glycan is attached to the nitrogen atom of the asparagine residue. See, e.g., Drickamer K, Taylor M E (2006), Introduction to Glycobiology (2nd ed.). Oxford University Press, USA. N-linked glycosylation motifs occur in the human IgM heavy chain constant regions of SEQ ID NO: 1 or SEQ ID NO: 2 starting at positions 46 (“N1”), 209 (“N2”), 272 (“N3”), 279 (“N4”), and 440 (“N5”). These five motifs are conserved in non-human primate IgM heavy chain constant regions, and four of the five are conserved in the mouse IgM heavy chain constant region. See FIG. 2. As provided elsewhere herein, each of these sites in the human IgM heavy chain constant region, except for N4, can be mutated to prevent glycosylation at that site, while still allowing IgM expression and assembly into a hexamer or pentamer.

Each IgM heavy chain constant region can be associated with a binding domain, e.g., an antigen-binding domain, e.g., a scFv or VHH, or a subunit of an antigen-binding domain, e.g., a VH region. In certain embodiments the binding domain can be a non-antibody binding domain, e.g., a receptor ectodomain, a ligand or receptor-binding fragment thereof, a cytokine or receptor-binding fragment thereof, a growth factor or receptor binding fragment thereof, a neurotransmitter or receptor binding fragment thereof, a peptide or protein hormone or receptor binding fragment thereof, an immune checkpoint modulator ligand or receptor-binding fragment thereof, or a receptor-binding fragment of an extracellular matrix protein. See, e.g., PCT Publication No. WO 202000867, which is incorporated herein by reference in its entirety.

Five IgM binding units can form a complex with an additional small polypeptide chain (the J-chain), or a functional fragment, variant, or derivative thereof, to form a pentameric IgM antibody or IgM-like antibody. The precursor form of the human J-chain is presented as SEQ ID NO: 19. The signal peptide extends from amino acid 1 to about amino acid 22 of SEQ ID NO: 19, and the mature human J-chain extends from about amino acid 23 to amino acid 159 of SEQ ID NO: 19. The mature human J-chain includes the amino acid sequence SEQ ID NO: 20.

Exemplary variant and modified J-chains are provided elsewhere herein. Without the J-chain, an IgM antibody or IgM-like antibody typically assembles into a hexamer, comprising up to twelve antigen-binding domains. With a J-chain, an IgM antibody or IgM-like antibody typically assembles into a pentamer, comprising up to ten antigen-binding domains, or more, if the J-chain is a modified J-chain comprising one or more heterologous polypeptides comprising additional antigen-binding domain(s). The assembly of five or six IgM binding units into a pentameric or hexameric IgM antibody or IgM-like antibody is thought to involve the Cμ4 and tailpiece domains. See, e.g., Braathen, R., et al., J. Biol. Chem. 277:42755-42762 (2002). Accordingly, a pentameric or hexameric IgM antibody provided in this disclosure typically includes at least the Cμ4 and/or tailpiece domains (also referred to herein collectively as Cμ4-tp). A “multimerizing fragment” of an IgM heavy chain constant region thus includes at least the Cμ4-tp domains. An IgM heavy chain constant region can additionally include a Cμ3 domain or a fragment thereof, a Cμ2 domain or a fragment thereof, a Cμ1 domain or a fragment thereof, and/or other IgM heavy chain domains. In certain embodiments, an IgM-derived binding molecule, e.g., an IgM antibody, IgM-like antibody, or other IgM-derived binding molecule as provided herein can include a complete IgM heavy GO chain constant domain, e.g., SEQ ID NO: 1 or SEQ ID NO: 2, or a variant, derivative, or analog thereof, e.g., as provided herein.

In certain embodiments, the disclosure provides a pentameric IgM antibody, IgM-like antibody, or other IgM-derived binding molecule comprising five bivalent binding units, where each binding unit includes two IgM heavy chain constant regions or multimerizing fragments or variants thereof, each associated with an antigen-binding domain or subunit thereof. In certain embodiments, the two IgM heavy chain constant regions are human heavy chain constant regions.

In some embodiments, the multimeric binding molecules are hexameric and comprise six bivalent binding units or variants or fragments thereof. In some embodiments, the multimeric binding molecules are hexameric and comprise six bivalent binding units or variants or fragments thereof, and where each binding unit comprises two IgM heavy chain constant regions or multimerizing fragments or variants thereof.

An IgM heavy chain constant region can include one or more of a Cμ1 domain or fragment or variant thereof, a Cμ2 domain or fragment or variant thereof, a Cμ3 domain or fragment or variant thereof, a Cμ4 domain or fragment or variant thereof, and/or a tail piece (tp) or fragment or variant thereof, provided that the constant region can serve a desired function in the IgM or IgM-like antibody, e.g., associate with second IgM constant region to form a binding unit with one, two, or more antigen-binding domain(s), and/or associate with other binding units (and in the case of a pentamer, a J-chain) to form a hexamer or a pentamer. In certain embodiments the two IgM heavy chain constant regions or fragments or variants thereof within an individual binding unit each comprise a Cμ4 domain or fragment or variant thereof, a tailpiece (tp) or fragment or variant thereof, or a combination of a Cμ4 domain and a tp or fragment or variant thereof. In certain embodiments the two IgM heavy chain constant regions or fragments or variants thereof within an individual binding unit each further comprise a Cμ3 domain or fragment or variant thereof, a Cμ2 domain or fragment or variant thereof, a Cμ1 domain or fragment or variant thereof, or any combination thereof.

In some embodiments, the binding units of the IgM or IgM-like antibody comprise two light chains. In some embodiments, the binding units of the IgM or IgM-like antibody comprise two fragments of light chains. In some embodiments, the light chains are kappa light chains. In some embodiments, the light chains are lambda light chains. In some embodiments, each binding unit comprises two immunoglobulin light chains each comprising a VL situated amino terminal to an immunoglobulin light chain constant region.

Where the IgM antibody, IgM-like antibody, or other IgM-derived binding molecule provided herein is pentameric, the IgM antibody, IgM-like antibody, or other IgM-derived binding molecule typically further includes a J-chain, or functional fragment or variant thereof. In certain embodiments, the J-chain is a modified J-chain or variant thereof that further comprises one or more heterologous moieties attached thereto, as described elsewhere herein. In certain embodiments the J-chain can be mutated to affect, e.g., enhance, the serum half-life of the IgM antibody, IgM-like antibody, or other IgM-derived binding molecule provided herein, as discussed elsewhere herein. In certain embodiments the J-chain can be mutated to affect glycosylation, as discussed elsewhere herein.

An IgM heavy chain constant region can include one or more of a CO domain or fragment or variant thereof, a Cμ2 domain or fragment or variant thereof, a Cμ3 domain or fragment or variant thereof, and/or a Cμ4 domain or fragment or variant thereof, provided that the constant region can serve a desired function in the an IgM antibody, IgM-like antibody, or other IgM-derived binding molecule, e.g., associate with second IgM constant region to form a binding unit with one, two, or more antigen-binding domain(s), and/or associate with other binding units (and in the case of a pentamer, a J-chain) to form a hexamer or a pentamer. In certain embodiments the two IgM heavy chain constant regions or fragments or variants thereof within an individual binding unit each comprise a Cμ4 domain or fragment or variant thereof, a tailpiece (tp) or fragment or variant thereof, or a combination of a Cμ4 domain and a TP or fragment or variant thereof. In certain embodiments the two IgM heavy chain constant regions or fragments or variants thereof within an individual binding unit each further comprise a Cμ3 domain or fragment or variant thereof, a Cμ2 domain or fragment or variant thereof, a Cμ1 domain or fragment or variant thereof, or any combination thereof.

Modified J-Chains

In certain embodiments, the J-chain of a pentameric IgM-derived binding molecule, e.g., an IgM antibody or IgM-like antibody as provided herein can be modified, e.g., by introduction of a heterologous moiety, or two or more heterologous moieties, e.g., polypeptides, without interfering with the ability of the IgM antibody, IgM-like antibody, or other IgM-derived binding molecule to assemble and bind to its binding target(s). See U.S. Pat. Nos. 9,951,134 and 10,618,978, and U.S. Patent Application Publication No. US-2019-0185570, each of which is incorporated herein by reference in its entirety. Accordingly, IgM or IgM-like antibodies as provided herein, including multispecific IgM or IgM-like antibodies as described elsewhere herein, can comprise a modified J-chain or functional fragment or variant thereof comprising a heterologous moiety, e.g., a heterologous polypeptide, introduced, e.g., fused or chemically conjugated, into the J-chain or fragment or variant thereof. In certain embodiments the heterologous moiety can be a peptide or polypeptide sequence fused in frame to the J-chain or chemically conjugated to the J-chain or fragment or variant thereof. In certain embodiments, the heterologous polypeptide is fused to the J-chain or functional fragment thereof via a peptide linker, e.g., a peptide linker, typically consisting of least 5 amino acids, but no more than 25 amino acids. In certain embodiments, the peptide linker consists of GGGGS (SEQ ID NO: 27), GGGGSGGGGS (SEQ ID NO: 28), GGGGSGGGGSGGGGS (SEQ ID NO: 29), GGGGSGGGGSGGGGSGGGGS (SEQ ID NO: 30), or GGGGSGGGGSGGGGSGGGGSGGGGS (SEQ ID NO: 31). In certain embodiments the heterologous moiety can be a chemical moiety conjugated to the J-chain. Heterologous moieties to be attached to a J-chain can include, without limitation, a binding moiety, e.g., an antibody or antigen-binding fragment thereof, e.g., a single chain Fv (scFv) molecule, a cytokine, e.g., IL-2 or IL-15 (see, e.g., PCT Application No. PCT/US2020/046379, which is incorporated herein by reference in its entirety), a stabilizing peptide that can increase the half-life of the IgM antibody, IgM-like antibody, or other IgM-derived binding molecule, or a chemical moiety such as a polymer or a cytotoxin. In some embodiments, heterologous moiety comprises a stabilizing peptide that can increase the half-life of the binding molecule, e.g., human serum albumin (HSA) or an HSA binding molecule.

In some embodiments, a modified J-chain can comprise an antigen-binding domain that can include without limitation a polypeptide (including small peptides) capable of specifically binding to a target antigen. In certain embodiments, an antigen-binding domain associated with a modified J-chain can be an antibody or an antigen-binding fragment thereof, as described elsewhere herein. In certain embodiments the antigen-binding domain can be a scFv antigen-binding domain or a single-chain antigen-binding domain derived, e.g., from a camelid or condricthoid antibody. The antigen-binding domain can be introduced into the J-chain at any location that allows the binding of the antigen-binding domain to its binding target without interfering with J-chain function or the function of an associated IgM or IgA antibody. Insertion locations include but are not limited to at or near the C-terminus, at or near the N-terminus or at an internal location that, based on the three-dimensional structure of the J-chain, is accessible. In certain embodiments, the antigen-binding domain can be introduced into the mature human J-chain of SEQ ID NO: 20 between cysteine residues 92 and 101 of SEQ ID NO: 20. In a further embodiment, the antigen-binding domain can be introduced into the human J-chain of SEQ ID NO: 20 at or near a glycosylation site. In a further embodiment, the antigen-binding domain can be introduced into the human J-chain of SEQ ID NO: 20 within about 10 amino acid residues from the C-terminus, or within about 10 amino acids from the N-terminus.

In certain embodiments, the J-chain of the IgM antibody, IgM-like antibody or other IgM-derived binding molecule as provided herein is a variant J-chain that comprises one or more amino acid substitutions that can alter, e.g., the serum half-life of an IgM antibody, IgM-like antibody, IgA antibody, IgA-like antibody, or IgM- or IgA-derived binding molecule provided herein. For example certain amino acid substitutions, deletions, or insertions can result in the IgM-derived binding molecule exhibiting an increased serum half-life upon administration to a subject animal relative to a reference IgM-derived binding molecule that is identical except for the one or more single amino acid substitutions, deletions, or insertions in the variant J-chain, and is administered using the same method to the same animal species. In certain embodiments the variant J-chain can include one, two, three, or four single amino acid substitutions, deletions, or insertions relative to the reference J-chain.

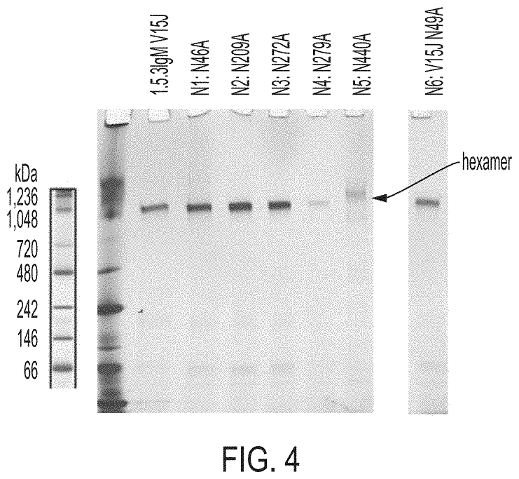

In some embodiments, the multimeric binding molecule can comprise a variant J-chain sequence, such as a variant sequence described herein with reduced glycosylation or reduced binding to one or more polymeric Ig receptors (e.g., pIgR, Fc alpha-mu receptor (FcαμR), or Fc mu receptor (FcμR)). See, e.g., PCT Publication No. WO 2019/169314, which is incorporated herein by reference in its entirety. In certain embodiments, the J-chain of the IgM antibody, IgM-like antibody or other IgM-derived binding molecule as provided herein comprises an amino acid substitution at the amino acid position corresponding to amino acid Y102 of the mature wild-type human J-chain (SEQ ID NO: 20). By “an amino acid corresponding to amino acid Y102 of the mature wild-type human J-chain” is meant the amino acid in the sequence of the J-chain of any species which is homologous to Y102 in the human J-chain See PCT Publication No. WO 2019/169314, which is incorporated herein by reference in its entirety. The position corresponding to Y102 in SEQ ID NO: 20 is conserved in the J-chain amino acid sequences of at least 43 other species. See FIG. 4 of U.S. Pat. No. 9,951,134, which is incorporated by reference herein. Certain mutations at the position corresponding to Y102 of SEQ ID NO: 20 can inhibit the binding of certain immunoglobulin receptors, e.g., the human or murine Fcαμ receptor, the murine Fcμ receptor, and/or the human or murine polymeric Ig receptor (pIg receptor) to an IgM pentamer comprising the mutant J-chain. IgM antibodies, IgM-like antibodies, and other IgM-derived binding molecules comprising a mutation at the amino acid corresponding to Y102 of SEQ ID NO: 20 have an improved serum half-life when administered to an animal than a corresponding antibody, antibody-like molecule or binding molecule that is identical except for the substitution, and which is administered to the same species in the same manner. In certain embodiments, the amino acid corresponding to Y102 of SEQ ID NO: 20 can be substituted with any amino acid. In certain embodiments, the amino acid corresponding to Y102 of SEQ ID NO: 20 can be substituted with alanine (A), serine (S) or arginine (R). In a particular embodiment, the amino acid corresponding to Y102 of SEQ ID NO: 20 can be substituted with alanine. In a particular embodiment the J-chain or functional fragment or variant thereof is a variant human J-chain referred to herein as “J*,” and comprises the amino acid sequence SEQ ID NO: 21.

Glycovariant IgM-Derived Binding Molecules

This disclosure provides an isolated IgM-derived binding molecule, e.g., an IgM antibody, IgM-like antibody, or other IgM-derived binding molecule, that includes at least one, at least two, at least three, at least four, at least five, at least six, at least seven, at least eight, at least nine, at least ten, at least eleven, or twelve variant IgM-derived heavy chain(s). As provided by the disclosure, the variant IgM-derived heavy chain(s) include a variant IgM heavy chain constant region, which can be a variant of a full-length IgM heavy chain constant region, a multimerizing fragment of an IgM heavy chain constant region, or a hybrid constant region that includes at least the minimal portion of an IgM heavy chain constant region required for multimerization, associated with a binding domain, e.g., an antibody antigen-binding domain, that specifically binds to a target of interest. The binding domain that binds to a target, can be, e.g., an antigen-binding domain or a subunit of an antigen-binding domain, e.g., the heavy chain variable region (VH) of an antibody. This disclosure relates to binding molecules that bind to any target of interest.

A variant IgM heavy chain constant region or variant IgM heavy chain constant regions as provided herein include alterations that affect glycosylation of the binding molecule, e.g., asparagine (N)-linked glycosylation. For example, the variant IgM heavy chain constant region(s) can include, e.g., one or more single amino acid insertions, deletions, or substitutions, that disrupt, e.g., prevent glycosylation, at one or more, two or more, three or more, or four of the five naturally-occurring asparagine(N)-linked glycosylation motifs (in the case of a human IgM heavy chain constant region) of the variant IgM heavy chain constant region is mutated to prevent glycosylation at that motif, and wherein an N-linked glycosylation motif comprises the amino acid sequence N-X₁-S/T, wherein N is asparagine, X₁ is any amino acid except proline, and S/T is serine or threonine. Human and non-human primate IgM heavy chain constant regions typically have five N-linked glycosylation motifs, where the mouse IgM heavy chain constant region typically has four N-linked glycosylation motifs. See FIG. 2.

IgM-derived binding molecules with alterations that affect glycosylation of the binding molecule can alter, e.g., improve certain physiologic, pharmacokinetic, or pharmacodynamic properties of the binding molecule. For example, such binding molecules can exhibit improved serum half-life, and/or allow for a more homogeneous antibody preparation during expression and manufacturing. Accordingly, such binding molecules can be incorporated into safer, more effective, and easier to manufacture biopharmaceuticals.

As provided herein, the variant IgM heavy chain constant region can be derived from a human IgM heavy chain constant region (e.g., SEQ ID NO: 1 or SEQ ID NO: 2) comprising five N-linked glycosylation motifs N-X₁-S/T starting at amino acid positions corresponding to amino acid 46 (motif N1), amino acid 209 (motif N2), amino acid 272 (motif N3), amino acid 279 (motif N4), and amino acid 440 (motif N5) of SEQ ID NO: 1 (allele IGHM*03) or SEQ ID NO: 2 (allele IGHM*04). The variant IgM heavy chain constant region can likewise be derived, e.g., from other human IgM alleles, from non-human primate IgM heavy chain constant regions or from IgM heavy chain constant regions of other species, e.g., rodent IgM heavy chain constant regions, e.g., mouse IgM heavy chain constant regions. The five N-linked glycosylation motifs in the human IgM heavy chain constant region, N1-N5, are conserved in other primate species, but in the mouse IgM heavy chain constant region, the N-linked glycosylation motif at position N3 is not conserved. See FIG. 2.

In certain embodiments, at least one, at least two, at least three, or at least four of the N-X₁-S/T motifs corresponding to motif N1, motif N2, motif N3, and/or motif N5 comprises an amino acid insertion, deletion, or substitution that prevents glycosylation at that motif. Prevention of glycosylation can be accomplished by eliminating the asparagine residue, or substituting it with a non-asparagine residue, or by eliminating the serine or threonine residue at the third position in the motif or substituting the serine or threonine residue with a non-serine or threonine residue. Prevention of glycosylation at the motif can also be accomplished by inserting a proline residue at position X₁ of the motif.

Accordingly, an IgM-derived binding molecule, e.g., an IgM antibody, IgM-like antibody, or other IgM-derived binding molecule as provided herein can include an amino acid insertion, deletion, or substitution at any of the N, X₁, or S/T positions of motif N1, motif N2, motif N3, motif N5, or any combination of two or more, three or more, or all four of motifs N1, N2, N3, or N5, wherein the amino acid insertion, deletion, or substitution prevents glycosylation at that motif.

In certain embodiments, an IgM-derived binding molecule, e.g., an IgM antibody, IgM-like antibody, or other IgM-derived binding molecule as provided herein can include an amino acid substitution at an amino acid position corresponding to amino acid N46, N209, N272, or N440 of SEQ ID NO: 1 or SEQ ID NO: 2 or an amino acid substitution at N46, N209, N272, or N440 of SEQ ID NO: 1 or SEQ ID NO: 2 wherein the substituted amino acid is any amino acid. As used herein, “an amino acid position corresponding to a particular amino acid in a sequence can be an amino acid in a homologous sequence, e.g., a conserved motif in a non-human primate heavy chain constant region, or in another allele of a human IgM constant region. In certain embodiments, an IgM-derived binding molecule, e.g., an IgM antibody, IgM-like antibody, or other IgM-derived binding molecule as provided herein can include an amino acid substitution at an amino acid position corresponding to amino acid S48, S211, S274, or S442 of SEQ ID NO: 1 or SEQ ID NO: 2 or an amino acid substitution at S48, S211, S274, or S442 of SEQ ID NO: 1 or SEQ ID NO: 2, wherein the substituted amino acid is any amino acid except threonine, or any combination of two or more, three or more, or four or more of the amino acid substitutions.

For example, an IgM-derived binding molecule, e.g., an IgM antibody, IgM-like antibody, or other IgM-derived binding molecule as provided herein can include an amino acid substitution corresponding to N46X₂, N46A, N46D, N46Q, N46K, 548X₃, S48A, N229X₂, N229A, N229D, N229Q, N229K, S231X₃, S231A, N272X₂, N272A, N272D, N272Q, N272K, S274X₃, S274A, N440X₂, N440A, N440D, N449Q, N449K, S242X₃, or S424A of SEQ ID NO: 1 or SEQ ID NO: 2, or any combination of two or more, three or more, or four or more of the amino acid substitutions, where X₂ is any amino acid and X₃ is any amino acid except threonine. The person of ordinary skill in the art will readily understand that additional amino acid substitutions, deletions, and/or insertions can likewise prevent N-linked glycosylation at a given motif

In certain embodiments, the variant IgM heavy chain constant region of the IgM-derived binding molecule, e.g., an IgM antibody, IgM-like antibody, or other IgM-derived binding molecule is a variant human IgM constant region comprising the amino acid sequence SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, or SEQ ID NO: 18. In each of these sequences, X191 can be G or S.

The variant IgM heavy chain constant region of an IgM-derived binding molecule, e.g., an IgM antibody, IgM-like antibody, or other IgM-derived binding molecule as provided herein can be further mutated to introduce at least one new asparagine(N)-linked glycosylation motif into the variant IgM heavy chain constant region, wherein the at least one new N-linked glycosylation motif is introduced at a site in the variant IgM heavy chain constant region that is not naturally glycosylated in an IgM antibody. Such new N-linked glycosylation motifs can improve the physical, pharmacokinetic, or pharmacodynamic properties of the IgM-derived binding molecule by, e.g., improving serum half-life, improving manufacturing yield, or providing more consistency to the glycans carried by the binding molecule. In certain embodiments, the new N-linked glycosylation motif can be introduced at a position in the variant IgM heavy chain constant region that corresponds to the position of an N-linked glycosylation motif present in a different immunoglobulin isotype. See, e.g., the alignments in FIG. 1. In certain embodiments the different immunoglobulin isotype is a human immunoglobulin isotype selected from the group consisting of human IgG1 (e.g., SEQ ID NO: 34), human IgG2 (e.g., SEQ ID NO: 35), human IgG3 (e.g., SEQ ID NO: 36), human IgG4 (e.g., SEQ ID NO: 37), human IgA1 (e.g., SEQ ID NO: 38), human IgA2 (e.g., SEQ ID NO: 39), human IgD (e.g., SEQ ID NO: 40), and human IgE (e.g., SEQ ID NO: 41). These sequences are presented below. The person of ordinary skill in the art will readily understand that allelic variants of these sequences exist and are included in this disclosure.

An IgM-derived binding molecule as provided herein includes at least one, at least two, at least three, at least four, at least five, at least six, at least seven, at least eight, at least nine, at least ten, at least eleven, or twelve glycovariant IgM heavy chain constant regions associated with a binding domain or subunit thereof, e.g., an antibody antigen-binding domain, e.g., a scFv, a VHH or the VH subunit of an antibody antigen-binding domain, that specifically binds to a target of interest. In certain embodiments, the target is a target epitope, a target antigen, a target cell, a target organ, or a target virus. Targets can include, without limitation, tumor antigens, other oncologic targets, immuno-oncologic targets such as immune checkpoint inhibitors, infectious disease targets, such as viral antigens expressed on the surface of infected cells, target antigens involved in blood-brain-barrier transport, target antigens involved in neurodegenerative diseases and neuroinflammatory diseases, and any combination thereof. Exemplary targets and binding domains that bind to such targets are provided elsewhere herein, and can be found in, e.g., U.S. Patent Application Publication No. US-2019-0100597, PCT Publication Nos. WO 2017/059387 (and related U.S. Publication No. US-2019-0185570), WO/2017/196867, WO 2018/017888, WO 2018/017889, WO 2018/017761, WO 2018/017763, WO 2018/187702, WO2019165340, WO 2019/169314, or WO 2020086745, PCT Application Nos. PCT/US2020/046379 or PCT/US2020/046335, or U.S. Pat. Nos. 9,951,134, 9,938,347, 8,377,435, 9,458,241, 9,409,976, 10,351,631, 10,570,191, 10,604,559, or 10,618,978. Each of these applications and/or patents is incorporated herein by reference in its entirety.

In certain embodiments the target is a tumor-specific antigen, i.e., a target antigen that is largely expressed only on tumor or cancer cells, or that may be expressed only at undetectable levels in normal healthy cells of an adult. In certain embodiments the target is a tumor-associated antigen, i.e., a target antigen that is expressed on both healthy and cancerous cells but is expressed at much higher density on cancerous cells than on normal healthy cells. Exemplary tumor-specific and tumor-associated antigens include, without limitation, B-cell maturation antigen (BCMA), CD19, CD20, epidermal growth factor receptor (EGFR), human epidermal growth factor receptor 2 (HER2, also called ErbB2), HERS (ErbB3), receptor tyrosine-protein kinase ErbB4, cytotoxic T-lymphocyte antigen 4 (CTLA4), programmed cell death protein 1 (PD-1), Programmed death-ligand 1 (PD-L1), vascular endothelial growth factor (VEGF), VEGF receptor-1 (VEGFR1), VEGFR2, CD52, CD30, prostate-specific membrane antigen (PSMA), CD38, ganglioside GD2, self-ligand receptor of the signaling lymphocytic activation molecule family member 7 (SLAMF7), platelet-derived growth factor receptor A (PDGFRA), CD22, FLT3 (CD135), CD123, MUC-16, carcinoembryonic antigen-related cell adhesion molecule 1 (CEACAM-1), mesothelin, tumor-associated calcium signal transducer 2 (Trop-2), glypican-3 (GPC-3), human blood group H type 1 trisaccharide (Globo-H), sialyl Tn antigen (STn antigen), or CD33. The skilled person will understand that these target antigens appear in the literature by a number of different names, but that these well-known therapeutic targets can be easily identified using databases available online, e.g., EXPASY.org.

Other tumor associated and/or tumor-specific antigens include, without limitation: DLL4, Notch1, Notch2, Notch3, Notch4, JAG1, JAG2, c-Met, IGF-1R, Patched, Hedgehog family polypeptides, WNT family polypeptides, FZD1, FZD2, FZD3, FZD4, FZD5, FZD6, FZD7, FZD8, FZD9, FZD10, LRP5, LRP6, IL-6, TNFalpha, IL-23, IL-17, CD80, CD86, CD3, CEA, Muc16, PSCA, CD44, c-Kit, DDR1, DDR2, RSPO1, RSPO2, RSPO3, RSPO4, BMP family polypeptides, BMPR1a, BMPR1b, or a TNF receptor superfamily protein such as TNFR1 (DR1), TNFR2, TNFR1/2, CD40 (p50), Fas (CD95, Apo1, DR2), CD30, 4-1BB (CD137, ILA), TRAILR1 (DR4, Apo2), DR5 (TRAILR2), TRAILR3 (DcR1), TRAILR4 (DcR2), OPG (OCIF), TWEAKR (FN14), LIGHTR (HVEM), DcR3, DR3, EDAR, or XEDAR.

In certain embodiments, the IgM-derived binding molecule, e.g., IgM antibody, IgM-like antibody, or other IgM-derived binding molecule is a pentameric or a hexameric IgM antibody, IgM-like antibody, or other IgM-derived binding molecule that includes five or six bivalent IgM binding units, respectively. According to certain embodiments, each binding unit includes two glycovariant IgM heavy chains as described herein, each having a VH situated amino terminal to the variant IgM constant region, and two immunoglobulin light chains each having a light chain variable domain (VL) situated amino terminal to an immunoglobulin light chain constant region, e.g., a kappa or lambda constant region. The provided VH and VL combine to form an antigen-binding domain that specifically binds to the target of interest. In certain embodiments, the five or six IgM binding units are identical.

In those embodiments where the IgM-derived binding molecule is pentameric, it can further include a J-chain, or functional fragment thereof, or a functional variant thereof, as described elsewhere herein. For example, the J-chain can be a mature human J-chain that includes the amino acid sequence SEQ ID NO: 20 or a functional fragment thereof, or a functional variant thereof. As persons of ordinary skill in the art will recognize, “a functional fragment” or a “functional variant” in this context includes those fragment and variant that can associate with IgM binding units, e.g., IgM heavy chain constant regions to form a pentameric IgM antibody.

In certain embodiments, the J-chain of a pentameric IgM-derived binding molecule, e.g., an IgM antibody, IgM-like antibody, or other IgM-derived binding molecule as provided herein is a functional variant J-chain that includes one or more single amino acid substitutions, deletions, or insertions relative to a reference J-chain identical to the variant J-chain except for the one or more single amino acid substitutions, deletions, or insertions. For example certain amino acid substitutions, deletions, or insertions can result in the IgM-derived binding molecule exhibiting an increased serum half-life upon administration to a subject animal relative to a reference IgM-derived binding molecule that is identical except for the one or more single amino acid substitutions, deletions, or insertions in the variant J-chain, and is administered in the same way to the same animal species. In certain embodiments the variant J-chain can include one, two, three, or four single amino acid substitutions, deletions, or insertions relative to the reference J-chain.

As described in detail elsewhere herein, in certain embodiments the variant J-chain or functional fragment thereof of a pentameric IgM-derived binding molecule as provided herein comprises an amino acid substitution at the amino acid position corresponding to amino acid Y102 of the wild-type mature human J-chain (SEQ ID NO: 20). Y102 can be substituted with any amino acid, for example alanine. In certain embodiments the variant human J-chain can include the amino acid sequence SEQ ID NO: 21, referred to herein as “J*”.

The J-chain or fragment of a pentameric IgM-derived binding molecule, e.g., an IgM antibody, IgM-like antibody, or other IgM-derived binding molecule as provided herein, having either a variant or wild type amino acid sequence, can be a “modified J-chain” that further include a heterologous moiety, wherein the heterologous moiety is fused or conjugated to the J-chain or fragment or variant thereof. Exemplary, but non-limiting heterologous moieties are provided, e.g., in U.S. Pat. Nos. 9,951,134 and 10,618,978, and in U.S. Patent Application Publication No. 2019/0185570, which are incorporated herein by reference. In certain embodiments, the heterologous moiety is a polypeptide fused to or within the J-chain or fragment or variant thereof. The heterologous polypeptide can in some instances be fused to or within the J-chain or fragment or variant thereof via a peptide linker. Any suitable linker can be used, for example the peptide linker can include at least 5 amino acids, at least ten amino acids, and least 20 amino acids, at least 30 amino acids or more, and so on. In certain embodiments the peptide linker includes no more than 25 amino acids. In certain embodiments the peptide linker can consist of 5 amino acids, 10 amino acids, 15 amino acids, 20 amino acids, or 25 amino acids. In certain embodiments the peptide linker comprises glycines and serines, e.g., (GGGGS)n, where N can be 1, 2, 3, 4, 5, or more (SEQ ID NO: 84). In certain embodiments, the peptide linker consists of GGGGS (SEQ ID NO: 27), GGGGSGGGGS (SEQ ID NO: 28), GGGGSGGGGSGGGGS (SEQ ID NO: 29), GGGGSGGGGSGGGGSGGGGS (SEQ ID NO: 30), or GGGGSGGGGSGGGGSGGGGSGGGGS (SEQ ID NO: 31). In certain embodiments, the heterologous polypeptide can be fused to the N-terminus of the J-chain or fragment or variant thereof, the C-terminus of the J-chain or fragment or variant thereof, or to both the N-terminus and C-terminus of the J-chain or fragment or variant thereof. In certain embodiments the heterologous polypeptide can be fused internally within the J-chain. In certain embodiments, the heterologous polypeptide can be a binding domain, e.g., an antigen binding domain. For example, the heterologous polypeptide can be an antibody, a subunit of an antibody, or an antigen-binding fragment of an antibody, e.g., a scFv fragment. In certain embodiments, the binding domain, e.g., scFv fragment can bind to an effector cell, e.g., a T cell or an NK cell. In certain embodiments the binding domain, e.g., scFv fragment can specifically bind to CD3 on cytotoxic T cells, e.g., to CD3ε. In certain specific embodiments, the modified J-chain of a pentameric IgM-derived binding molecule as provided herein comprises the amino acid sequence SEQ ID NO: 24 (V15J), SEQ ID NO: 25 (V15J*), SEQ ID NO: 26 (V15J N49D) or a J-chain comprising an anti-CD3c scFv antigen-binding domain comprising the six complementarity-determining region of murine antibody SP34, the VHCDR1, VHCDR2, VHCDR3, VLCDR1, VLCDR2, and VLCDR3 amino acid sequences SEQ ID NO: 48, SEQ ID NO: 49, SEQ ID NO: 50, SEQ ID NO: 52, SEQ ID NO: 53, and SEQ ID NO: 54, respectively, e.g., the modified J-chain SJ*, comprising the amino acid sequence SEQ ID NO: 55 or an anti-CD3c scFv antigen-binding domain comprising the VHCDR1, VHCDR2, VHCDR3, VLCDR1, VLCDR2, and VLCDR3 amino acid sequences SEQ ID NO: 57, SEQ ID NO: 59, SEQ ID NO: 62, SEQ ID NO: 65, SEQ ID NO: 67, and SEQ ID NO: 69; SEQ ID NO: 57, SEQ ID NO: 59, SEQ ID NO: 62, SEQ ID NO: 65, SEQ ID NO: 67, and SEQ ID NO: 70; SEQ ID NO: 58, SEQ ID NO: 60, SEQ ID NO: 63, SEQ ID NO: 66, SEQ ID NO: 68, and SEQ ID NO: 71; SEQ ID NO: 58, SEQ ID NO: 61, SEQ ID NO: 63, SEQ ID NO: 66, SEQ ID NO: 68, and SEQ ID NO: 72; SEQ ID NO: 58, SEQ ID NO: 61, SEQ ID NO: 64, SEQ ID NO: 66, SEQ ID NO: 68, and SEQ ID NO: 73, respectively, e.g., the VH and VL of SEQ ID NO: 74 and SEQ ID NO: 75, SEQ ID NO: 76 and SEQ ID NO: 77, SEQ ID NO: 78 and SEQ ID NO: 79, SEQ ID NO: 80 and SEQ ID NO: 81, or SEQ ID NO: 82 and SEQ ID NO: 83, respectively.

IgM-Derived Binding Molecules with Enhanced Serum Half-Life

Certain IgM-derived binding molecules, e.g., IgM antibodies, IgM-like antibodies, or other IgM-derived binding molecules as provided herein, in addition to the glycosylation mutations described herein can be further engineered to have enhanced serum half-life. Exemplary IgM heavy chain constant region mutations that can enhance serum half-life of an IgM-derived binding molecule are disclosed in WO 2019/169314, which is incorporated by reference herein in its entirety. For example, in addition to one or more of the glycosylation mutations described elsewhere herein, a variant IgM heavy chain constant region of an IgM-derived binding molecule as provided herein can include an amino acid substitution at an amino acid position corresponding to amino acid S401, E402, E403, R344, and/or E345 of a wild-type human IgM constant region (e.g., SEQ ID NO: 1 or SEQ ID NO: 2). By “an amino acid corresponding to amino acid S401, E402, E403, R344, and/or E345 of a wild-type human IgM constant region” is meant the amino acid in the sequence of the IgM constant region of any species which is homologous to S401, E402, E403, R344, and/or E345 in the human IgM constant region. In certain embodiments, the amino acid corresponding to S401, E402, E403, R344, and/or E345 of SEQ ID NO: 1 or SEQ ID NO: 2 can be substituted with any amino acid, e.g., alanine.

Wild-type J-chains typically include one N-linked glycosylation site. In certain embodiments, a variant J-chain or functional fragment thereof of a pentameric IgM-derived binding molecule as provided herein includes a mutation within the asparagine(N)-linked glycosylation motif N-X₁-S/T, e.g., starting at the amino acid position corresponding to amino acid 49 (motif N6) of the mature human J-chain (SEQ ID NO: 20) or J* (SEQ ID NO: 21), wherein N is asparagine, X₁ is any amino acid except proline, and S/T is serine or threonine, and wherein the mutation prevents glycosylation at that motif. As demonstrated in PCT Publication No. WO 2019/169314, mutations preventing glycosylation at this site can result in the IgM-derived binding molecule, e.g., an IgM antibody, IgM-like antibody, or other IgM-derived binding molecule as provided herein, exhibiting an increased serum half-life upon administration to a subject animal relative to a reference IgM-derived binding molecule that is identical except for the mutation or mutations preventing glycosylation in the variant J-chain, and is administered in the same way to the same animal species.

For example, in certain embodiments the variant J-chain or functional fragment thereof of a pentameric IgM-derived binding molecule as provided herein can include an amino acid substitution at the amino acid position corresponding to amino acid N49 or amino acid S51 SEQ ID NO: 20, provided that the amino acid corresponding to S51 is not substituted with threonine (T), or wherein the variant J-chain comprises amino acid substitutions at the amino acid positions corresponding to both amino acids N49 and S51 of SEQ ID NO: 20. In certain embodiments, the position corresponding to N49 of SEQ ID NO: 20 is substituted with any amino acid, e.g., alanine (A), glycine (G), threonine (T), serine (S) or aspartic acid (D). In a particular embodiment, the position corresponding to N49 of SEQ ID NO: 20 can be substituted with alanine (A). In a particular embodiment, the J-chain of a pentameric IgM-derived binding molecule as provided herein is a variant human J-chain and has the amino acid sequence SEQ ID NO: 22. In another particular embodiment, the position corresponding to N49 of SEQ ID NO: 20 can be substituted with aspartic acid (D). In a particular embodiment, the J-chain of a pentameric IgM-derived binding molecule as provided herein is a variant human J-chain and has the amino acid sequence SEQ ID NO: 23.

Variant Human IgM Constant Regions with Reduced CDC Activity

Certain IgM-derived binding molecules, e.g., IgM antibodies, IgM-like antibodies, or other IgM-derived binding molecules as provided herein, in addition to the glycosylation mutations described herein can be further engineered to exhibit reduced complement-dependent cytotoxicity (CDC) activity to cells in the presence of complement, relative to a reference IgM antibody or IgM-like antibody with a corresponding reference human IgM constant region identical, except for the mutations conferring reduced CDC activity. These CDC mutations can be combined with any of the mutations to block N-linked glycosylation and/or to confer increased serum half-life as provided herein. By “corresponding reference human IgM constant region” is meant a human IgM constant region or portion thereof, e.g., a Cμ3 domain, that is identical to the variant IgM constant region except for the modification or modifications in the constant region affecting CDC activity. In certain embodiments, the variant human IgM constant region includes one or more amino acid substitutions, e.g., in the Cμ3 domain, relative to a wild-type human IgM constant region as described, e.g., in PCT Publication No. WO/2018/187702, which is incorporated herein by reference in its entirety. Assays for measuring CDC are well known to those of ordinary skill in the art, and exemplary assays are described e.g., in PCT Publication No. WO/2018/187702.

In certain embodiments, a variant human IgM constant region conferring reduced

CDC activity includes an amino acid substitution corresponding to the wild-type human IgM constant region at position L310, P311, P313, and/or K315 of SEQ ID NO: 1 (human IgM constant region allele IGHM*03) or SEQ ID NO: 2 (human IgM constant region allele IGHM*04). In certain embodiments, a variant human IgM constant region conferring reduced CDC activity includes an amino acid substitution corresponding to the wild-type human IgM constant region at position P311 of SEQ ID NO: 1 or SEQ ID NO: 2. In other embodiments the variant IgM constant region as provided herein contains an amino acid substitution corresponding to the wild-type human IgM constant region at position P313 of SEQ ID NO: 1 or SEQ ID NO: 2. In other embodiments the variant IgM constant region as provided herein contains a combination of substitutions corresponding to the wild-type human IgM constant region at positions P311 of SEQ ID NO: 1 or SEQ ID NO: 2 and/or P313 of SEQ ID NO: 1 or SEQ ID NO: 2. These proline residues can be independently substituted with any amino acid, e.g., with alanine, serine, or glycine. In certain embodiments, a variant human IgM constant region conferring reduced CDC activity includes an amino acid substitution corresponding to the wild-type human IgM constant region at position K315 of SEQ ID NO: 1 or SEQ ID NO: 2. The lysine residue can be independently substituted with any amino acid, e.g., with alanine, serine, glycine, or aspartic acid. In certain embodiments, a variant human IgM constant region conferring reduced CDC activity includes an amino acid substitution corresponding to the wild-type human IgM constant region at position K315 of SEQ ID NO: 1 or SEQ ID NO: 2 with aspartic acid. In certain embodiments, a variant human IgM constant region conferring reduced CDC activity includes an amino acid substitution corresponding to the wild-type human IgM constant region at position L310 of SEQ ID NO: 1 or SEQ ID NO: 2. The lysine residue can be independently substituted with any amino acid, e.g., with alanine, serine, glycine, or aspartic acid. In certain embodiments, a variant human IgM constant region conferring reduced CDC activity includes an amino acid substitution corresponding to the wild-type human IgM constant region at position L310 of SEQ ID NO: 1 or SEQ ID NO: 2 with aspartic acid.

Polynucleotides, Vectors, and Host Cells

The disclosure further provides a polynucleotide, e.g., an isolated, recombinant, and/or non-naturally-occurring polynucleotide, comprising a nucleic acid sequence that encodes a polypeptide subunit of an IgM-derived binding molecule, e.g., an IgM antibody, IgM-like antibody, or other IgM-derived binding molecule as provided herein. By “polypeptide subunit” is meant a portion of a binding molecule, binding unit, IgM antibody, IgM-like antibody, or antigen-binding domain that can be independently translated. Examples include, without limitation, an antibody variable domain, e.g., a VH or a VL, a J chain, a secretory component, a single chain Fv, an antibody heavy chain, an antibody light chain, an antibody heavy chain constant region, an antibody light chain constant region, and/or any fragment, variant, or derivative thereof.

In certain embodiments, the polypeptide subunit can comprise a variant IgM-derived heavy chain as provided herein, which comprises a variant IgM heavy chain constant region, where at least one asparagine(N)-linked glycosylation motif of the variant IgM heavy chain constant region is mutated to prevent glycosylation at that motif. The variant IgM heavy chain constant region can be fused to a binding domain, e.g., an antigen-binding domain or a subunit thereof, e.g., to the VH portion of an antigen-binding domain, all as provided herein. In certain embodiments the polynucleotide can encode a polypeptide subunit comprising a variant human IgM-derived heavy chain constant region. For example, the IgM-derived heavy chain polypeptide subunit can comprise the amino acid sequence of any of SEQ ID NOs: 3-18.

In certain embodiments, the polypeptide subunit can include an antibody VL portion of an antigen-binding domain as described elsewhere herein. In certain embodiments the polypeptide subunit can include an antibody light chain constant region, e.g., a human antibody light chain constant region, or fragment thereof, which can be fused to the C-terminal end of a VL.

In certain embodiments the polypeptide subunit can include a J-chain, a modified J-chain, or any functional fragment or variant thereof, as provided herein. In certain embodiments the polypeptide subunit can comprise a human J-chain or functional fragment or variant thereof, including modified J-chains. In certain embodiments the J-chain polypeptide subunit can comprise the amino acid sequence of any of SEQ ID NOs: 19-26 or 55.

In certain embodiments a polynucleotide as provided herein, e.g., an expression vector such as a plasmid, can include a nucleic acid sequence encoding one polypeptide subunit, e.g., a variant IgM-derived heavy chain, a light chain, or a J-chain, or can include two or more nucleic acid sequences encoding two or more or all three polypeptide subunits of an IgM-derived binding molecule as provided herein. Alternatively, the nucleic acid sequences encoding the three polypeptide subunits can be on separate polynucleotides, e.g., separate expression vectors. The disclosure provides such single or multiple expression vectors. The disclosure also provides one or more host cells encoding the provided polynucleotide(s) or expression vector(s).

Thus, in certain embodiments, to form the antigen-binding domains the nucleic acid sequences encoding the variable regions of antibodies can be inserted into expression vector templates for IgM-derived structures, in particular those encoding variant IgM heavy chain constant regions as provided herein, for example any of SEQ ID NOs: 3-18, and can be further combined with a polynucleotide encoding a J-chain or functional fragment or variant thereof as provided herein, e.g., encoding any of SEQ ID NOs: 19-26 or 55, and a light chain, thereby creating an IgM-derived binding molecule having five or six binding units in which glycosylation is impaired at one or more N-linked glycosylation motifs, as described elsewhere herein. In brief, nucleic acid sequences encoding the heavy and light chain variable domain sequences can be synthesized or amplified from existing molecules and inserted into one or more vectors in the proper orientation and in frame such that upon expression, the vector will yield the desired full length heavy or light chain. Vectors useful for these purposes are known in the art. Such vectors can also comprise enhancer and other sequences needed to achieve expression of the desired chains. Multiple vectors or single vectors can be used. This vector or these vectors can be transfected into host cells and then the variant IgM-derived heavy chain and/or light chains and/or J-chain or functional fragment or variant thereof are expressed, IgM-derived binding molecules are assembled, and can then be isolated and/or purified. Upon expression the chains form fully functional multimeric IgM-derived binding molecules, e.g., IgM antibodies, IgM-like antibodies, or other IgM-derived binding molecules as provided herein, possessing enhanced serum half-life. The expression and purification processes can be performed at commercial scale, if needed.

The disclosure further provides a composition comprising two or more polynucleotides, where the two or more polynucleotides collectively can encode an IgM-derived binding molecule with altered glycosylation as described above. In certain embodiments the composition can include a polynucleotide encoding a variant IgM-derived heavy chain or multimerizing fragment thereof as provided elsewhere herein, for example any of SEQ ID NOs: 3-18, where the IgM-like heavy chain further includes a binding domain, e.g., an antigen-binding domain or a subunit thereof, e.g., a VH domain. The composition can further include a polynucleotide encoding a light chain or fragment thereof, e.g., a human kappa or lambda light chain that comprises at least a VL of an antigen-binding domain. A polynucleotide composition as provided can further include a polynucleotide encoding a J-chain or functional fragment or variant thereof as provided herein, for example any of SEQ ID NOs: 19-26 or 55. In certain embodiments the polynucleotides making up a composition as provided herein can be situated on two, three, or more separate vectors, e.g., expression vectors. Such vectors are provided by the disclosure. In certain embodiments two or more of the polynucleotides making up a composition as provided herein can be situated on a single vector, e.g., an expression vector. Such a vector is provided by the disclosure.

The disclosure further provides a host cell, e.g., a prokaryotic or eukaryotic host cell, comprising a polynucleotide or two or more polynucleotides encoding an IgM-derived binding molecule as provided herein, or any subunit thereof, a polynucleotide composition as provided herein, or a vector or two, three, or more vectors that collectively encode the IgM-derived binding molecule as provided herein, or any subunit thereof.

In a related embodiment, the disclosure provides a method of producing an IgM-derived binding molecule with reduced glycosylation as provided by this disclosure, where the method comprises culturing a host cell as provided herein and recovering the IgM-derived binding molecule.

Methods of Use

The disclosure further provides a method of treating a disease or disorder in a subject in need of treatment, comprising administering to the subject a therapeutically effective amount of an IgM-derived binding molecule, e.g., an IgM antibody, IgM-like antibody, or other IgM-derived binding molecule as provided herein. IgM-derived binding molecules with reduced glycosylation as provided by this disclosure can result in more homogeneous therapeutic compositions by simplifying the number glycoforms on the binding molecule and by making the characteristics of the sugars attached to the binding molecule more uniform, e.g., a more complete addition of sialic acid groups to the glycans. Such improvements to homogeneity can confer greater ease in manufacturing and also greater safety upon the binding molecules relative to a reference IgM-derived binding molecule that is identical except for the reduction in glycosylation. Moreover, an IgM-derived binding molecule with reduced glycosylation can exhibit increased serum half-life relative to a reference IgM-derived binding molecule that is identical except for the reduction in glycosylation. By “therapeutically effective dose or amount” or “effective amount” is intended an amount of an IgM-derived binding molecule that when administered brings about a positive therapeutic response with respect to treatment of subject.

Effective doses of compositions for, e.g., treatment of cancer vary depending upon many different factors, including means of administration, target site, physiological state of the subject, whether the subject is human or an animal, other medications administered, and whether treatment is prophylactic or therapeutic. Usually, the subject is a human, but non-human mammals including transgenic mammals can also be treated. Treatment dosages can be titrated using routine methods known to those of skill in the art to optimize safety and efficacy.

The subject to be treated can be any animal, e.g., mammal, in need of treatment, in certain embodiments, the subject is a human subject.

In its simplest form, a preparation to be administered to a subject is an IgM-derived binding molecule as provided herein, or a multimeric antigen-binding fragment thereof, administered in conventional dosage form, which can be combined with a pharmaceutical excipient, carrier or diluent as described elsewhere herein.

The compositions of the disclosure can be administered by any suitable method, e.g., parenterally, intraventricularly, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

Pharmaceutical Compositions and Administration Methods

Methods of preparing and administering an IgM-derived binding molecule, e.g., an

IgM antibody, IgM-like antibody, or other IgM-derived binding molecule as provided herein to a subject in need thereof are well known to or are readily determined by those skilled in the art in view of this disclosure. In certain embodiments, the form of administration would be a solution for injection, in particular for intratumoral, intravenous, or intraarterial injection or drip. A suitable pharmaceutical composition can comprise a buffer (e.g. acetate, phosphate or citrate buffer), a surfactant (e.g. polysorbate), optionally a stabilizer agent (e.g. human albumin), etc.

The disclosed IgM-derived binding molecule can be formulated so as to facilitate administration and promote stability of the active agent. Pharmaceutical compositions accordingly can comprise a pharmaceutically acceptable, non-toxic, sterile carrier such as physiological saline, non-toxic buffers, preservatives and the like. A pharmaceutically effective amount of an IgM-derived binding molecule as provided herein means an amount sufficient to achieve effective binding to a target and to achieve a therapeutic benefit. Suitable formulations are described in Remington's Pharmaceutical Sciences, e.g., 21^{st} Edition (Lippincott Williams & Wilkins) (2005).

Certain pharmaceutical compositions provided herein can be orally administered in an acceptable dosage form including, e.g., capsules, tablets, aqueous suspensions or solutions. Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

The amount of an IgM-derived binding molecule that can be combined with carrier materials to produce a single dosage form will vary depending, e.g., upon the subject treated and the particular mode of administration. The composition can be administered as a single dose, multiple doses or over an established period of time in an infusion. Dosage regimens also can be adjusted to provide the optimum desired response (e.g., a therapeutic or prophylactic response).

This disclosure also provides for the use of an IgM-derived binding molecule as provided herein in the manufacture of a medicament for treating, preventing, or managing disease, e.g., cancer. This disclosure also provides an IgM-derived binding molecule as provided herein for use in treating, preventing, or managing disease, e.g., cancer.

This disclosure employs, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Green and Sambrook, ed. (2012) Molecular Cloning A Laboratory Manual (4th ed.; Cold Spring Harbor Laboratory Press); Sambrook et al., ed. (1992) Molecular Cloning: A Laboratory Manual, (Cold Springs Harbor Laboratory, NY); D. N. Glover and B. D. Hames, eds., (1995) DNA Cloning 2d Edition (IRL Press), Volumes 1-4; Gait, ed. (1990) Oligonucleotide Synthesis (IRL Press); Mullis et al. U.S. Pat. No. 4,683,195; Hames and Higgins, eds. (1985) Nucleic Acid Hybridization (IRL Press); Hames and Higgins, eds. (1984) Transcription And Translation (IRL Press); Freshney (2016) Culture Of Animal Cells, 7th Edition (Wiley-Blackwell); Woodward, J., Immobilized Cells And Enzymes (IRL Press) (1985); Perbal (1988) A Practical Guide To Molecular Cloning; 2d Edition (Wiley-Interscience); Miller and Calos eds. (1987) Gene Transfer Vectors For Mammalian Cells, (Cold Spring Harbor Laboratory); S. C. Makrides (2003) Gene Transfer and Expression in Mammalian Cells (Elsevier Science); Methods in Enzymology, Vols. 151-155 (Academic Press, Inc., N.Y.); Mayer and Walker, eds. (1987) Immunochemical Methods in Cell and Molecular Biology (Academic Press, London); Weir and Blackwell, eds.; and in Ausubel et al. (1995) Current Protocols in Molecular Biology (John Wiley and Sons).

General principles of antibody engineering are set forth, e.g., in Strohl, W. R., and L. M. Strohl (2012), Therapeutic Antibody Engineering (Woodhead Publishing). General principles of protein engineering are set forth, e.g., in Park and Cochran, eds. (2009), Protein Engineering and Design (CDC Press). General principles of immunology are set forth, e.g., in: Abbas and Lichtman (2017) Cellular and Molecular Immunology 9th Edition (Elsevier). Additionally, standard methods in immunology known in the art can be followed, e.g., in Current Protocols in Immunology (Wiley Online Library); Wild, D. (2013), The Immunoassay Handbook 4th Edition (Elsevier Science); Greenfield, ed. (2013), Antibodies, a Laboratory Manual, 2d Edition (Cold Spring Harbor Press); and Ossipow and Fischer, eds., (2014), Monoclonal Antibodies: Methods and Protocols (Humana Press).

All of the references cited above, as well as all references cited herein, are incorporated herein by reference in their entireties.

The following examples are offered by way of illustration and not by way of limitation.

### EXAMPLES

### Example 1: Construction and Characterization of IgM Glycovariants

The N-linked glycosylation sites of all human immunoglobulins are compared in

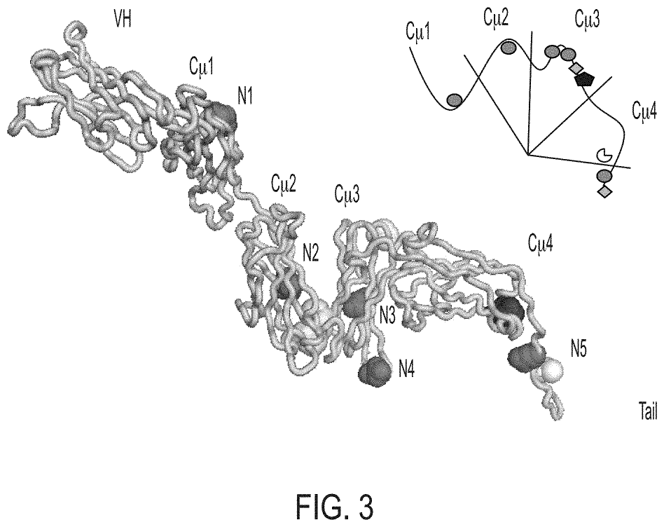

FIG. 1A and FIG. 1B. The N-linked glycosylation sites of IgM antibodies of various different species are shown in FIG. 2A and FIG. 2B. A space-filling model of a human IgM heavy chain is shown in FIG. 3. The five N-linked glycosylation motifs are depicted as N1 in the Cμ1 domain (N46 of SEQ ID NO: 1 or SEQ ID NO: 2), N2 in the Cμ2 domain (N209 of SEQ ID NO: 1 or SEQ ID NO: 2), N3 in the Cμ3 domain (N272 of SEQ ID NO: 1 or SEQ ID NO: 2), N4 in the Cμ3 domain (N279 of SEQ ID NO: 1 or SEQ ID NO: 2), and N5 in the tail piece domain (N440 of SEQ ID NO: 1 or SEQ ID NO: 2).

DNA variants encoding modified human IgM constant regions with single alanine or aspartic acid mutations of the asparagine (N) residues in the five N-linked glycosylation motifs present in human IgM constant region of SEQ ID NO: 2, were designed and submitted to a commercial vendor for synthesis. Exemplary plasmid constructs that can express wild-type or modified human pentameric or hexameric IgM antibodies comprising the wild-type or modified IgM constant regions, and that can specifically bind to CD20, were produced by the following method.

DNA fragments encoding the VH and VL regions of 1.5.3 (SEQ ID NOs 32 and 33, respectively, see U.S. Application Publication No. 2019-0100597) and the various single asparagine to alanine mutations or asparagine to aspartic acid mutations at N1-N5 were synthesized by a commercial vendor for subcloning into heavy chain and light chain expression vectors by standard molecular biology techniques.

Plasmid constructs encoding the IgM heavy chains, light chains, and a modified J-chain (V15J, SEQ ID NO: 24) were cotransfected into CHO cells, and cells that express glycovariant anti-CD20 IgM antibodies were selected, all according to standard methods. A sixth single alanine mutation was made in then-linked glycosylation motif in the V15J J-chain at N49 (N6) and coupled with a wild-type IgM.

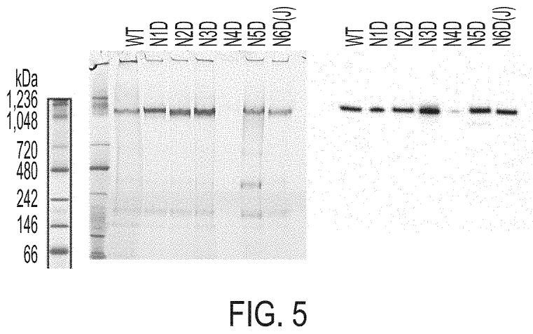

Antibodies present in the cell supernatants were recovered using Capture Select IgM (Catalog 2890.05, BAC, Thermo Fisher) according to the manufacturer's protocol. Antibodies were evaluated on SDS PAGE under non-reducing conditions to show assembly as previously described, e.g., in PCT Publication No. WO 2016/141303. The alanine mutants are shown in FIG. 4 and the aspartic acid mutants are shown in FIG. 5, along with a western blot reacted with anti-J-chain antibody. As shown in FIG. 4, variant IgMs with single alanine mutations at N1, N2, and N3 expressed and assembled as well as the corresponding wild-type IgM (1.5.3IgM V15J), where the variant IgM with single alanine mutations at N4 showed reduced expression, and the variant IgM with the single alanine mutation at N5 assembled as a hexamer. The IgM with the single alanine mutation at N6 also expressed and assembled properly. As shown in FIG. 5, variant IgMs with single aspartic acid mutations at N1, N2, N3, N5, and N6 expressed and assembled properly as pentamers, where the mutation at N4 did not express or assemble.

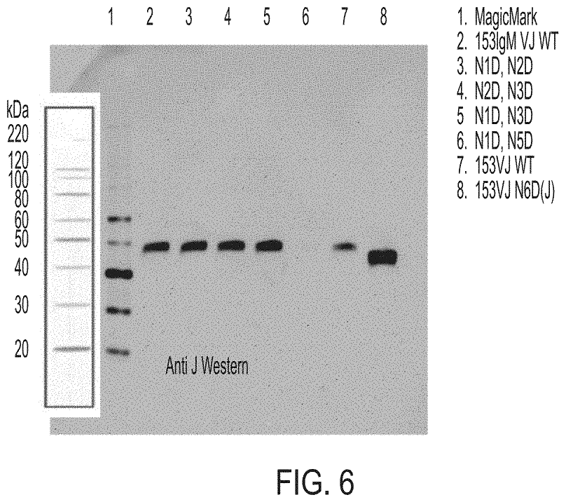

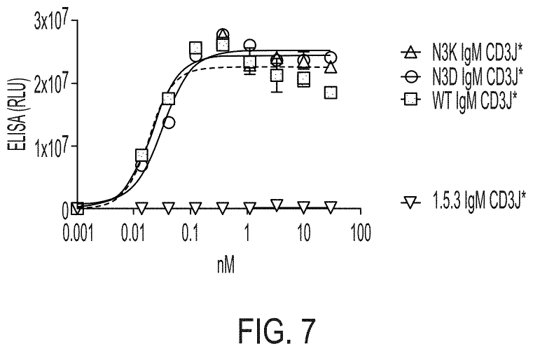

Next, selected double aspartic acid mutants were constructed as described above, and were evaluated on SDS PAGE under non-reducing conditions to show assembly as previously described, e.g., in PCT Publication No. WO 2016/141303. To show proper assembly of the IgM binding units with a J-chain, these mutants were evaluated by western blot using an anti-J-chain antibody. The results are shown in FIG. 6. Double mutants at N1D and N2D, N2D and N3D, and N1D and N3D all expressed and assembled properly as pentamers, but a double mutant at N1D and N5D either did not express or assembled as hexamer without a J-chain as the construct did not react with the anti-J-chain antibody.

### Example 2: Complement Dependent Cytotoxicity

The 1.5.3IgM V15J, 1.5.3IgM V15J N1A, 1.5.3IgM V15J N2A, and 1.5.3IgM V15J N3A antibodies generated in Example 1 were compared using a Complement Dependent Cytotoxicity (CDC) assay.

The Raji cell line (ATCC cat. #CCL-86), which expresses CD20, was used to determine the CDC efficacy of each of the antibodies. 50,000 cells were seeded in a 96-well plate. Cells were treated with serially diluted antibody. Human serum complement (Quidel cat. #A113) was added to each well at a final concentration of 10%. The reaction mixtures were incubated at 37° C. for 4 hours. Cell Titer Glo reagent (Promega cat. #G7572) was added at a volume equal to the volume of culture medium present in each well. The plate was shaken for 2 minutes, incubated for 10 minutes at room temperature, and luminescence was measured on a luminometer. There was no significant difference in CDC activity between the antibodies tested (data not shown).

### Example 3: T-Cell Activation Assay

The 1.5.3IgM V15J, 1.5.3IgM V15J N1A, 1.5.3IgM V15J N2A, and 1.5.3IgM V15J N3A antibodies generated in Example 1 were compared using a T-cell activation assay.

Engineered Jurkat T-cells (Promega CS176403) and RPMI8226 cells (ATCC CCL-155) were cultured in RPMI (Invitrogen) supplemented with 10% Fetal Bovine Serum (Invitrogen). Serial dilutions of antibody were incubated with 7500 RPMI8226 cells in 20 μL in a white 384 well assay plate for 2 h at 37° C. with 5% CO₂. The engineered Jurkat cells (25000) were added to mixture to final volume of 40 μL. The mixture was incubated for 5h at 37° C. with 5% CO₂. The cell mixtures were then mixed with 20 μL lysis buffer containing luciferin (Promega, Cell Titer Glo) to measure luciferase reporter activity. Light output was measured by EnVision plate reader. EC50 was determined by 4 parameter curve fit using Prism software. There was no significant difference in T-cell activation between the antibodies tested (data not shown).

### Example 4: ELISA Binding

Antibodies were generated using WT Human IgM constant region (SEQ ID NO: 1), N3D IgM constant region (SEQ ID NO: 8), or N3K IgM constant region (SEQ ID NO: 56) fused to exemplary binding domains and comprising anti-CD3J*. The ability of the antibodies to bind the target of the exemplary binding domain was compared to the 1.5.3 WT IgM VJ* generated in Example 1.

96-well white polystyrene ELISA plates (Pierce 15042) were coated with 100 per well of 10 μg/mL or 0.3 μg/mL target protein overnight at 4° C. Plates were then washed with 0.05% PBS-Tween and blocked with 2% BSA-PBS. After blocking, 100 μL of serial dilutions of the antibody was added to the wells and incubated at room temperature for 2 hours. The plates were then washed and incubated with HRP conjugated mouse anti-human kappa (Southern Biotech, 9230-05. 1:6000 diluted in 2% BSA-PBS) for 30 min. After 10 final washes using 0.05% PBS-Tween, the plates were read out using SuperSignal chemiluminescent substrate (ThermoFisher, 37070). Luminescent data were collected on an EnVision plate reader (Perkin-Elmer) and analyzed with GraphPad Prism using a 4-parameter logistic model.

The results are shown in FIG. 8. The binding was comparable between the exemplary antibodies. The anti-CD20 IgM antibody did not bind the target.

The breadth and scope of the present disclosure should not be limited by any of the above-described exemplary embodiments but should be defined only in accordance with the following claims and their equivalents.

TABLE 2
Sequences in the Disclosure

| SEQ | Short Name | |
|---|---|---|
| ID NO | or Citation | Sequence |
| 1 | Human IgM | GSASAPTLFPLVSCENSPSDTSSVAVGCLAQDFLPDSITFS |
| | Constant | WKYKNNSDISSTRGFPSVLRGGKYAATSQVLLPSKDVMQ |
| | region | GTDEHVVCKVQHPNGNKEKNVPLPVIAELPPKVSVFVPPR |
| | IMGT allele | DGFFGNPRKSKLICQATGFSPRQIQVSWLREGKQVGSGVT |
| | IGHM*03 | TDQVQAEAKESGPTTYKVTSTLTIKESDWLSQSMFTCRVD |
| | | HRGLTFQQNASSMCVPDQDTAIRVFAIPPSFASIFLTKSTKL |
| | | TCLVTDLTTYDSVTISWTRQNGEAVKTHTNISESHPNATFS |
| | | AVGEASICEDDWNSGERFTCTVTHTDLPSPLKQTISRPKGV |
| | | ALHRPDVYLLPPAREQLNLRESATITCLVTGFSPADVFVQ |
| | | WMQRGQPLSPEKYVTSAPMPEPQAPGRYFAHSILTVSEEE |
| | | WNTGETYTCVVAHEALPNRVTERTVDKSTGKPTLYNVSL |
| | | VMSDTAGTCY |
| 2 | Human IgM | GSASAPTLFPLVSCENSPSDTSSVAVGCLAQDFLPDSITFS |
| | Constant | WKYKNNSDISSTRGFPSVLRGGKYAATSQVLLPSKDVMQ |
| | region | GTDEHVVCKVQHPNGNKEKNVPLPVIAELPPKVSVFVPPR |
| | IMGT allele | DGFFGNPRKSKLICQATGFSPRQIQVSWLREGKQVGSGVT |
| | IGHM*04;. | TDQVQAEAKESGPTTYKVTSTLTIKESDWLGQSMFTCRVD |
| | | HRGLTFQQNASSMCVPDQDTAIRVFAIPPSFASIFLTKSTKL |
| | | TCLVTDLTTYDSVTISWTRQNGEAVKTHTNISESHPNATFS |
| | | AVGEASICEDDWNSGERFTCTVTHTDLPSPLKQTISRPKGV |
| | | ALHRPDVYLLPPAREQLNLRESATITCLVTGFSPADVFVQ |
| | | WMQRGQPLSPEKYVTSAPMPEPQAPGRYFAHSILTVSEEE |
| | | WNTGETYTCVVAHEALPNRVTERTVDKSTGKPTLYNVSL |
| | | VMSDTAGTCY |
| 3 | IgM N46A X191 | GSASAPTLFPLVSCENSPSDTSSVAVGCLAQDFLPDSITFS |
| | can be G or S | WKYKANSDISSTRGFPSVLRGGKYAATSQVLLPSKDVMQ |
| | | GTDEHVVCKVQHPNGNKEKNVPLPVIAELPPKVSVFVPPR |
| | | DGFFGNPRKSKLICQATGFSPRQIQVSWLREGKQVGSGVT |
| | | TDQVQAEAKESGPTTYKVTSTLTIKESDWLXQSMFTCRVD |
| | | HRGLTFQQNASSMCVPDQDTAIRVFAIPPSFASIFLTKSTKL |
| | | TCLVTDLTTYDSVTISWTRQNGEAVKTHTNISESHPNATFS |
| | | AVGEASICEDDWNSGERFTCTVTHTDLPSPLKQTISRPKGV |
| | | ALHRPDVYLLPPAREQLNLRESATITCLVTGFSPADVFVQ |
| | | WMQRGQPLSPEKYVTSAPMPEPQAPGRYFAHSILTVSEEE |
| | | WNTGETYTCVVAHEALPNRVTERTVDKSTGKPTLYNVSL |
| | | VMSDTAGTCY |
| 4 | IgM N46D X191 | GSASAPTLFPLVSCENSPSDTSSVAVGCLAQDFLPDSITFS |
| | can be G or S | WKYKDNSDISSTRGFPSVLRGGKYAATSQVLLPSKDVMQ |
| | | GTDEHVVCKVQHPNGNKEKNVPLPVIAELPPKVSVFVPPR |
| | | DGFFGNPRKSKLICQATGFSPRQIQVSWLREGKQVGSGVT |
| | | TDQVQAEAKESGPTTYKVTSTLTIKESDWLXQSMFTCRVD |
| | | HRGLTFQQNASSMCVPDQDTAIRVFAIPPSFASIFLTKSTKL |
| | | TCLVTDLTTYDSVTISWTRQNGEAVKTHTNISESHPNATFS |
| | | AVGEASICEDDWNSGERFTCTVTHTDLPSPLKQTISRPKGV |
| | | ALHRPDVYLLPPAREQLNLRESATITCLVTGFSPADVFVQ |
| | | WMQRGQPLSPEKYVTSAPMPEPQAPGRYFAHSILTVSEEE |
| | | WNTGETYTCVVAHEALPNRVTERTVDKSTGKPTLYNVSL |
| | | VMSDTAGTCY |
| 5 | IgM N209A | GSASAPTLFPLVSCENSPSDTSSVAVGCLAQDFLPDSITFS |
| | X191 can be G | WKYKNNSDISSTRGFPSVLRGGKYAATSQVLLPSKDVMQ |
| | or S | GTDEHVVCKVQHPNGNKEKNVPLPVIAELPPKVSVFVPPR |
| | | DGFFGNPRKSKLICQATGFSPRQIQVSWLREGKQVGSGVT |
| | | TDQVQAEAKESGPTTYKVTSTLTIKESDWLXQSMFTCRVD |
| | | HRGLTFQQAASSMCVPDQDTAIRVFAIPPSFASIFLTKSTKL |
| | | TCLVTDLTTYDSVTISWTRQNGEAVKTHTNISESHPNATFS |
| | | AVGEASICEDDWNSGERFTCTVTHTDLPSPLKQTISRPKGV |
| | | ALHRPDVYLLPPAREQLNLRESATITCLVTGFSPADVFVQ |
| | | WMQRGQPLSPEKYVTSAPMPEPQAPGRYFAHSILTVSEEE |
| | | WNTGETYTCVVAHEALPNRVTERTVDKSTGKPTLYNVSL |
| | | VMSDTAGTCY |
| 6 | IgM N209D | GSASAPTLFPLVSCENSPSDTSSVAVGCLAQDFLPDSITFS |
| | X191 can be G | WKYKNNSDISSTRGFPSVLRGGKYAATSQVLLPSKDVMQ |
| | or S | GTDEHVVCKVQHPNGNKEKNVPLPVIAELPPKVSVFVPPR |
| | | DGFFGNPRKSKLICQATGFSPRQIQVSWLREGKQVGSGVT |
| | | TDQVQAEAKESGPTTYKVTSTLTIKESDWLXQSMFTCRVD |
| | | HRGLTFQQDASSMCVPDQDTAIRVFAIPPSFASIFLTKSTKL |
| | | TCLVTDLTTYDSVTISWTRQNGEAVKTHTNISESHPNATFS |
| | | AVGEASICEDDWNSGERFTCTVTHTDLPSPLKQTISRPKGV |
| | | ALHRPDVYLLPPAREQLNLRESATITCLVTGFSPADVFVQ |
| | | WMQRGQPLSPEKYVTSAPMPEPQAPGRYFAHSILTVSEEE |
| | | WNTGETYTCVVAHEALPNRVTERTVDKSTGKPTLYNVSL |
| | | VMSDTAGTCY |
| 7 | IgM N272A | GSASAPTLFPLVSCENSPSDTSSVAVGCLAQDFLPDSITFS |
| | X191 can be G | WKYKNNSDISSTRGFPSVLRGGKYAATSQVLLPSKDVMQ |
| | or S | GTDEHVVCKVQHPNGNKEKNVPLPVIAELPPKVSVFVPPR |
| | | DGFFGNPRKSKLICQATGFSPRQIQVSWLREGKQVGSGVT |
| | | TDQVQAEAKESGPTTYKVTSTLTIKESDWLXQSMFTCRVD |
| | | HRGLTFQQNASSMCVPDQDTAIRVFAIPPSFASIFLTKSTKL |
| | | TCLVTDLTTYDSVTISWTRQNGEAVKTHTAISESHPNATFS |
| | | AVGEASICEDDWNSGERFTCTVTHTDLPSPLKQTISRPKGV |
| | | ALHRPDVYLLPPAREQLNLRESATITCLVTGFSPADVFVQ |
| | | WMQRGQPLSPEKYVTSAPMPEPQAPGRYFAHSILTVSEEE |
| | | WNTGETYTCVVAHEALPNRVTERTVDKSTGKPTLYNVSL |
| | | VMSDTAGTCY |
| 8 | IgM N272D | GSASAPTLFPLVSCENSPSDTSSVAVGCLAQDFLPDSITFS |
| | X191 can be G | WKYKNNSDISSTRGFPSVLRGGKYAATSQVLLPSKDVMQ |
| | or S | GTDEHVVCKVQHPNGNKEKNVPLPVIAELPPKVSVFVPPR |
| | | DGFFGNPRKSKLICQATGFSPRQIQVSWLREGKQVGSGVT |
| | | TDQVQAEAKESGPTTYKVTSTLTIKESDWLXQSMFTCRVD |
| | | HRGLTFQQNASSMCVPDQDTAIRVFAIPPSFASIFLTKSTKL |
| | | TCLVTDLTTYDSVTISWTRQNGEAVKTHTDISESHPNATFS |
| | | AVGEASICEDDWNSGERFTCTVTHTDLPSPLKQTISRPKGV |
| | | ALHRPDVYLLPPAREQLNLRESATITCLVTGFSPADVFVQ |
| | | WMQRGQPLSPEKYVTSAPMPEPQAPGRYFAHSILTVSEEE |
| | | WNTGETYTCVVAHEALPNRVTERTVDKSTGKPTLYNVSL |
| | | VMSDTAGTCY |
| 9 | IgM N279A | GSASAPTLFPLVSCENSPSDTSSVAVGCLAQDFLPDSITFS |
| | X191 can be G | WKYKNNSDISSTRGFPSVLRGGKYAATSQVLLPSKDVMQ |
| | or S | GTDEHVVCKVQHPNGNKEKNVPLPVIAELPPKVSVFVPPR |
| | | DGFFGNPRKSKLICQATGFSPRQIQVSWLREGKQVGSGVT |
| | | TDQVQAEAKESGPTTYKVTSTLTIKESDWLXQSMFTCRVD |
| | | HRGLTFQQNASSMCVPDQDTAIRVFAIPPSFASIFLTKSTKL |
| | | TCLVTDLTTYDSVTISWTRQNGEAVKTHTNISESHPAATFS |
| | | AVGEASICEDDWNSGERFTCTVTHTDLPSPLKQTISRPKGV |
| | | ALHRPDVYLLPPAREQLNLRESATITCLVTGFSPADVFVQ |
| | | WMQRGQPLSPEKYVTSAPMPEPQAPGRYFAHSILTVSEEE |
| | | WNTGETYTCVVAHEALPNRVTERTVDKSTGKPTLYNVSL |
| | | VMSDTAGTCY |
| 10 | IgM N279D | GSASAPTLFPLVSCENSPSDTSSVAVGCLAQDFLPDSITFS |
| | X191 can be G | WKYKNNSDISSTRGFPSVLRGGKYAATSQVLLPSKDVMQ |
| | or S | GTDEHVVCKVQHPNGNKEKNVPLPVIAELPPKVSVFVPPR |
| | | DGFFGNPRKSKLICQATGFSPRQIQVSWLREGKQVGSGVT |
| | | TDQVQAEAKESGPTTYKVTSTLTIKESDWLXQSMFTCRVD |
| | | HRGLTFQQNASSMCVPDQDTAIRVFAIPPSFASIFLTKSTKL |
| | | TCLVTDLTTYDSVTISWTRQNGEAVKTHTNISESHPDATFS |
| | | AVGEASICEDDWNSGERFTCTVTHTDLPSPLKQTISRPKGV |
| | | ALHRPDVYLLPPAREQLNLRESATITCLVTGFSPADVFVQ |
| | | WMQRGQPLSPEKYVTSAPMPEPQAPGRYFAHSILTVSEEE |
| | | WNTGETYTCVVAHEALPNRVTERTVDKSTGKPTLYNVSL |
| | | VMSDTAGTCY |
| 11 | IgM N440A | GSASAPTLFPLVSCENSPSDTSSVAVGCLAQDFLPDSITFS |
| | X191 can be G | WKYKNNSDISSTRGFPSVLRGGKYAATSQVLLPSKDVMQ |
| | or S | GTDEHVVCKVQHPNGNKEKNVPLPVIAELPPKVSVFVPPR |
| | | DGFFGNPRKSKLICQATGFSPRQIQVSWLREGKQVGSGVT |
| | | TDQVQAEAKESGPTTYKVTSTLTIKESDWLXQSMFTCRVD |
| | | HRGLTFQQNASSMCVPDQDTAIRVFAIPPSFASIFLTKSTKL |
| | | TCLVTDLTTYDSVTISWTRQNGEAVKTHTNISESHPNATFS |
| | | AVGEASICEDDWNSGERFTCTVTHTDLPSPLKQTISRPKGV |
| | | ALHRPDVYLLPPAREQLNLRESATITCLVTGFSPADVFVQ |
| | | WMQRGQPLSPEKYVTSAPMPEPQAPGRYFAHSILTVSEEE |
| | | WNTGETYTCVVAHEALPNRVTERTVDKSTGKPTLYAVSL |
| | | VMSDTAGTCY |
| 12 | IgM N440D | GSASAPTLFPLVSCENSPSDTSSVAVGCLAQDFLPDSITFS |
| | X191 can be G | WKYKNNSDISSTRGFPSVLRGGKYAATSQVLLPSKDVMQ |
| | or S | GTDEHVVCKVQHPNGNKEKNVPLPVIAELPPKVSVFVPPR |
| | | DGFFGNPRKSKLICQATGFSPRQIQVSWLREGKQVGSGVT |
| | | TDQVQAEAKESGPTTYKVTSTLTIKESDWLXQSMFTCRVD |
| | | HRGLTFQQNASSMCVPDQDTAIRVFAIPPSFASIFLTKSTKL |
| | | TCLVTDLTTYDSVTISWTRQNGEAVKTHTNISESHPNATFS |
| | | AVGEASICEDDWNSGERFTCTVTHTDLPSPLKQTISRPKGV |
| | | ALHRPDVYLLPPAREQLNLRESATITCLVTGFSPADVFVQ |
| | | WMQRGQPLSPEKYVTSAPMPEPQAPGRYFAHSILTVSEEE |
| | | WNTGETYTCVVAHEALPNRVTERTVDKSTGKPTLYDVSL |
| | | VMSDTAGTCY |
| 13 | N46D, N209D | GSASAPTLFPLVSCENSPSDTSSVAVGCLAQDFLPDSITFS |
| | X191 can be G | WKYKDNSDISSTRGFPSVLRGGKYAATSQVLLPSKDVMQ |
| | or S | GTDEHVVCKVQHPNGNKEKNVPLPVIAELPPKVSVFVPPR |
| | | DGFFGNPRKSKLICQATGFSPRQIQVSWLREGKQVGSGVT |
| | | TDQVQAEAKESGPTTYKVTSTLTIKESDWLXQSMFTCRVD |
| | | HRGLTFQQDASSMCVPDQDTAIRVFAIPPSFASIFLTKSTKL |
| | | TCLVTDLTTYDSVTISWTRQNGEAVKTHTNISESHPNATFS |
| | | AVGEASICEDDWNSGERFTCTVTHTDLPSPLKQTISRPKGV |
| | | ALHRPDVYLLPPAREQLNLRESATITCLVTGFSPADVFVQ |
| | | WMQRGQPLSPEKYVTSAPMPEPQAPGRYFAHSILTVSEEE |
| | | WNTGETYTCVVAHEALPNRVTERTVDKSTGKPTLYNVSL |
| | | VMSDTAGTCY |
| 14 | N209D, N272D | GSASAPTLFPLVSCENSPSDTSSVAVGCLAQDFLPDSITFS |
| | X191 can be G | WKYKDNSDISSTRGFPSVLRGGKYAATSQVLLPSKDVMQ |
| | or S | GTDEHVVCKVQHPNGNKEKNVPLPVIAELPPKVSVFVPPR |
| | | DGFFGNPRKSKLICQATGFSPRQIQVSWLREGKQVGSGVT |
| | | TDQVQAEAKESGPTTYKVTSTLTIKESDWLXQSMFTCRVD |
| | | HRGLTFQQDASSMCVPDQDTAIRVFAIPPSFASIFLTKSTKL |
| | | TCLVTDLTTYDSVTISWTRQNGEAVKTHTDISESHPNATFS |
| | | AVGEASICEDDWNSGERFTCTVTHTDLPSPLKQTISRPKGV |
| | | ALHRPDVYLLPPAREQLNLRESATITCLVTGFSPADVFVQ |
| | | WMQRGQPLSPEKYVTSAPMPEPQAPGRYFAHSILTVSEEE |
| | | WNTGETYTCVVAHEALPNRVTERTVDKSTGKPTLYNVSL |
| | | VMSDTAGTCY |
| 15 | N46D, N272D | GSASAPTLFPLVSCENSPSDTSSVAVGCLAQDFLPDSITFS |
| | X191 can be G | WKYKDNSDISSTRGFPSVLRGGKYAATSQVLLPSKDVMQ |
| | or S | GTDEHVVCKVQHPNGNKEKNVPLPVIAELPPKVSVFVPPR |
| | | DGFFGNPRKSKLICQATGFSPRQIQVSWLREGKQVGSGVT |
| | | TDQVQAEAKESGPTTYKVTSTLTIKESDWLXQSMFTCRVD |
| | | HRGLTFQQNASSMCVPDQDTAIRVFAIPPSFASIFLTKSTKL |
| | | TCLVTDLTTYDSVTISWTRQNGEAVKTHTDISESHPNATFS |
| | | AVGEASICEDDWNSGERFTCTVTHTDLPSPLKQTISRPKGV |
| | | ALHRPDVYLLPPAREQLNLRESATITCLVTGFSPADVFVQ |
| | | WMQRGQPLSPEKYVTSAPMPEPQAPGRYFAHSILTVSEEE |
| | | WNTGETYTCVVAHEALPNRVTERTVDKSTGKPTLYNVSL |
| | | VMSDTAGTCY |
| 16 | N46D, N440D; | GSASAPTLFPLVSCENSPSDTSSVAVGCLAQDFLPDSITFS |
| | X191 can be G | WKYKDNSDISSTRGFPSVLRGGKYAATSQVLLPSKDVMQ |
| | or S | GTDEHVVCKVQHPNGNKEKNVPLPVIAELPPKVSVFVPPR |
| | | DGFFGNPRKSKLICQATGFSPRQIQVSWLREGKQVGSGVT |
| | | TDQVQAEAKESGPTTYKVTSTLTIKESDWLXQSMFTCRVD |
| | | HRGLTFQQNASSMCVPDQDTAIRVFAIPPSFASIFLTKSTKL |
| | | TCLVTDLTTYDSVTISWTRQNGEAVKTHTNISESHPNATFS |
| | | AVGEASICEDDWNSGERFTCTVTHTDLPSPLKQTISRPKGV |
| | | ALHRPDVYLLPPAREQLNLRESATITCLVTGFSPADVFVQ |
| | | WMQRGQPLSPEKYVTSAPMPEPQAPGRYFAHSILTVSEEE |
| | | WNTGETYTCVVAHEALPNRVTERTVDKSTGKPTLYDVSL |
| | | VMSDTAGTCY |
| 17 | N46D, N209D, | GSASAPTLFPLVSCENSPSDTSSVAVGCLAQDFLPDSITFS |
| | N272D; X191 | WKYKDNSDISSTRGFPSVLRGGKYAATSQVLLPSKDVMQ |
| | can be G or S | GTDEHVVCKVQHPNGNKEKNVPLPVIAELPPKVSVFVPPR |
| | | DGFFGNPRKSKLICQATGFSPRQIQVSWLREGKQVGSGVT |
| | | TDQVQAEAKESGPTTYKVTSTLTIKESDWLXQSMFTCRVD |
| | | HRGLTFQQDASSMCVPDQDTAIRVFAIPPSFASIFLTKSTKL |
| | | TCLVTDLTTYDSVTISWTRQNGEAVKTHTDISESHPNATFS |
| | | AVGEASICEDDWNSGERFTCTVTHTDLPSPLKQTISRPKGV |
| | | ALHRPDVYLLPPAREQLNLRESATITCLVTGFSPADVFVQ |
| | | WMQRGQPLSPEKYVTSAPMPEPQAPGRYFAHSILTVSEEE |
| | | WNTGETYTCVVAHEALPNRVTERTVDKSTGKPTLYNVSL |
| | | VMSDTAGTCY |
| 18 | N46D, N209D, | GSASAPTLFPLVSCENSPSDTSSVAVGCLAQDFLPDSITFS |
| | N272D, N440D; | WKYKDNSDISSTRGFPSVLRGGKYAATSQVLLPSKDVMQ |
| | X191 can be G | GTDEHVVCKVQHPNGNKEKNVPLPVIAELPPKVSVFVPPR |
| | or S | DGFFGNPRKSKLICQATGFSPRQIQVSWLREGKQVGSGVT |
| | | TDQVQAEAKESGPTTYKVTSTLTIKESDWLXQSMFTCRVD |
| | | HRGLTFQQDASSMCVPDQDTAIRVFAIPPSFASIFLTKSTKL |
| | | TCLVTDLTTYDSVTISWTRQNGEAVKTHTDISESHPNATFS |
| | | AVGEASICEDDWNSGERFTCTVTHTDLPSPLKQTISRPKGV |
| | | ALHRPDVYLLPPAREQLNLRESATITCLVTGFSPADVFVQ |
| | | WMQRGQPLSPEKYVTSAPMPEPQAPGRYFAHSILTVSEEE |
| | | WNTGETYTCVVAHEALPNRVTERTVDKSTGKPTLYDVSL |
| | | VMSDTAGTCY |
| 19 | Precursor | MKNHLLFWGVLAVFIKAVHVKAQEDERIVLVDNKCKCARIT |
| | Human J Chain | SRIIRSSEDPNEDIVERNIRIIVPLNNRENISDPTSPLRTRF |
| | | VYHLSDLCKKCDPTEVELDNQIVTATQSNICDEDSATETC |
| | | YTYDRNKCYTAVVPLVYGGETKMVETALTPDACYPD |
| 20 | Mature Human | QEDERIVLVDNKCKCARITSRIIRSSEDPNEDIVERNIRIIVP |
| | J Chain | LNNRENISDPTSPLRTRFVYHLSDLCKKCDPTEVELDNQIV |
| | | TATQSNICDEDSATETCYTYDRNKCYTAVVPLVYGGETK |
| | | MVETALTPDACYPD |
| 21 | J Chain | QEDERIVLVDNKCKCARITSRIIRSSEDPNEDIVERNIRIIVP |
| | variant J* | LNNRENISDPTSPLRTRFVYHLSDLCKKCDPTEVELDNQIV |
| | | TATQSNICDEDSATETCATYDRNKCYTAVVPLVYGGETK |
| | | MVETALTPDACYPD |
| 22 | J Chain N49A | QEDERIVLVDNKCKCARITSRIIRSSEDPNEDIVERNIRIIVP |
| | mutation | LNNREAISDPTSPLRTRFVYHLSDLCKKCDPTEVELDNQIV |
| | | TATQSNICDEDSATETCYTYDRNKCYTAVVPLVYGGETK |
| | | MVETALTPDACYPD |
| 23 | J Chain N49D | QEDERIVLVDNKCKCARITSRIIRSSEDPNEDIVERNIRIIVP |
| | mutation | LNNREDISDPTSPLRTRFVYHLSDLCKKCDPTEVELDNQIV |
| | | TATQSNICDEDSATETCYTYDRNKCYTAVVPLVYGGETK |
| | | MVETALTPDACYPD |
| 24 | V15J (“WT”, | QVQLVQSGAEVKKPGASVKVSCKASGYTFISYTMHWVRQ |
| | or wild-type) | APGQGLEWMGYINPRSGYTHYNQKLKDKATLTADKSAST |
| | | AYMELSSLRSEDTAVYYCARSAYYDYDGFAYWGQGTLV |
| | | TVSSGGGGSGGGGSGGGGSDIQMTQSPSSLSASVGDRVTI |
| | | TCSASSSVSYMNWYQQKPGKAPKRLIYDTSKLASGVPSRF |
| | | SGSGSGTDFTLTISSLQPEDFATYYCQQWSSNPPTFGGGTK |
| | | LEIKGGGGSGGGGSGGGGSQEDERIVLVDNKCKCARITSRI |
| | | IRSSEDPNEDIVERNIRIIVPLNNRENISDPTSPLRTRFVYH |
| | | LSDLCKKCDPTEVELDNQIVTATQSNICDEDSATETCYTYDR |
| | | NKCYTAVVPLVYGGETKMVETALTPDACYPD |
| 25 | V15J* | QVQLVQSGAEVKKPGASVKVSCKASGYTFISYTMHWVRQ |
| | | APGQGLEWMGYINPRSGYTHYNQKLKDKATLTADKSAST |
| | | AYMELSSLRSEDTAVYYCARSAYYDYDGFAYWGQGTLV |
| | | TVSSGGGGSGGGGSGGGGSDIQMTQSPSSLSASVGDRVTI |
| | | TCSASSSVSYMNWYQQKPGKAPKRLIYDTSKLASGVPSRF |
| | | SGSGSGTDFTLTISSLQPEDFATYYCQQWSSNPPTFGGGTK |
| | | LEIKGGGGSGGGGSGGGGSQEDERIVLVDNKCKCARITSRI |
| | | IRSSEDPNEDIVERNIRIIVPLNNRENISDPTSPLRTRFVYH |
| | | LSDLCKKCDPTEVELDNQIVTATQSNICDEDSATETCATYDR |
| | | NKCYTAVVPLVYGGETKMVETALTPDACYPD |
| 26 | V15J (N49D) | QVQLVQSGAEVKKPGASVKVSCKASGYTFISYTMHWVRQ |
| | | APGQGLEWMGYINPRSGYTHYNQKLKDKATLTADKSAST |
| | | AYMELSSLRSEDTAVYYCARSAYYDYDGFAYWGQGTLV |
| | | TVSSGGGGSGGGGSGGGGSDIQMTQSPSSLSASVGDRVTI |
| | | TCSASSSVSYMNWYQQKPGKAPKRLIYDTSKLASGVPSRF |
| | | SGSGSGTDFTLTISSLQPEDFATYYCQQWSSNPPTFGGGTK |
| | | LEIKGGGGSGGGGSGGGGSQEDERIVLVDNKCKCARITSRI |
| | | IRSSEDPNEDIVERNIRIIVPLNNREDISDPTSPLRTRFVYH |
| | | LSDLCKKCDPTEVELDNQIVTATQSNICDEDSATETCYTYDR |
| | | NKCYTAVVPLVYGGETKMVETALTPDACYPD |
| 27 | 5-linker | GGGGS |
| 28 | 10 linker | GGGGSGGGGS |
| 29 | 15-linker | GGGGSGGGGSGGGGS |
| 30 | 20-linker | GGGGSGGGGSGGGGSGGGGS |
| 31 | 25 linker | GGGGSGGGGSGGGGSGGGGSGGGGS |
| 32 | 1.5.3 VH | EVQLVQSGAEVKKPGESLKISCKGSGYSFTSYWIGWVRQ |
| | | MPGKGLEWMGIIYPGDSDTRYSPSFQGQVTISADKSITTAY |
| | | LQWSSLKASDTAMYYCARHPSYGSGSPNFDYWGQGTLV |
| | | TVSS |
| 33 | 1.5.3 VL | DIVMTQTPLSSPVTLGQPASISCRSSQSLVYSDGNTYLSWL |
| | | QQRPGQPPRLLIYKISNRFSGVPDRFSGSGAGTDFTLKISRV |
| | | EAEDVGVYYCVQATQFPLTFGGGTKVEIK |
| 34 | human IgG1 | ASTKGPSVFPLAPSSKSTSGGTAALGCLVKDYFPEPVTVS |
| | heavy chain | WNSGALTSGVHTFPAVLQSSGLYSLSSVVTVPSSSLGTQT |
| | constant | YICNVNHKPSNTKVDKKVEPKSCDKTHTCPPCPAPELLGG |
| | region, e.g., | PSVFLFPPKPKDTLMISRTPEVTCVVVDVSHEDPEVKFNW |
| | 141-470 of | YVDGVEVHNAKTKPREEQYNSTYRVVSVLTVLHQDWLN |
| | GenBank | GKEYKCKVSNKALPAPIEKTISKAKGQPREPQVYTLPPSRD |
| | AIC63046.1 | ELTKNQVSLTCLVKGFYPSDIAVEWESNGQPENNYKTTPP |
| | | VLDSDGSFFLYSKLTVDKSRWQQGNVFSCSVMHEALHNH |
| | | YTQKSLSLSPGK |
| 35 | human IgG2 | ASTKGPSVFPLAPCSRSTSESTAALGCLVKDYFPEPVTVSW |
| | heavy chain | NSGALTSGVHTFPAVLQSSGLYSLSSVVTVPSSNFGTQTYT |
| | constant | CNVDHKPSNTKVDKTVERKCCVECPPCPAPPVAGPSVFLF |
| | region, e.g., | PPKPKDTLMISRTPEVTCVVVDVSHEDPEVQFNWYVDGV |
| | amino acids | EVHNAKTKPREEQFNSTFRVVSVLTVVHQDWLNGKEYKC |
| | 1-326 of | KVSNKGLPAPIEKTISKTKGQPREPQVYTLPPSREEMTKNQ |
| | GenBank | VSLTCLVKGFYPSDIAVEWESNGQPENNYKTTPPMLDSDG |
| | AXN93662.2 | SFFLYSKLTVDKSRWQQGNVFSCSVMHEALHNHYTQKSL |
| | | SLSPGK |
| 36 | Human IgG3 | ASTKGPSVFPLAPCSRSTSGGTAALGCLVKDYFPEPVTVS |
| | heavy chain | WNSGALTSGVHTFPAVLQSSGLYSLSSVVTVPSSSLGTQT |
| | constant | YTCNVNHKPSNTKVDKRVELKTPLGDTTHTCPRCPEPKSC |
| | region, e.g., | DTPPPCPRCPEPKSCDTPPPCPRCPEPKSCDTPPPCPRCPAP |
| | amino acids | ELLGGPSVFLFPPKPKDTLMISRTPEVTCVVVDVSHEDPEVQ |
| | 1 to 377 of | FKWYVDGVEVHNAKTKPREEQFNSTFRVVSVLTVLHQD |
| | GenBank | WLNGKEYKCKVSNKGLPAPIEKTISKTKGQPREPQVYTLP |
| | AXN93659.2 | PSREEMTKNQVSLTCLVKGFYPSDIAVEWESSGQPENNYN |
| | | TTPPMLDSDGSFFLYSKLTVDKSRWQQGNIFSCSVMHEAL |
| | | HNRFTQKSLSLSPGK |
| 37 | Human IgG4 | ASTKGPSVFPLAPCSRSTSESTAALGCLVKDYFPEPVTVSW |
| | heavy chain | NSGALTSGVHTFPAVLQSSGLYSLSSVVTVPSSSLGTKTYT |
| | constant | CNVDHKPSNTKVDKRVESKYGPPCPSCPAPEFLGGPSVFL |
| | region, e.g., | FPPKPKDTLMISRTPEVTCVVVDVSQEDPEVQFNWYVDG |
| | amino acids | VEVHNAKTKPREEQFNSTYRVVSVLTVLHQDWLNGKEY |
| | 1 to 327 of | KCKVSNKGLPSSIEKTISKAKGQPREPQVYTLPPSQEEMTK |
| | GenBank | NQVSLTCLVKGFYPSDIAVEWESNGQPENNYKTTPPVLDS |
| | sp\|P01861.1 | DGSFFLYSRLTVDKSRWQEGNVFSCSVMHEALHNHYTQK |
| | | SLSLSLGK |
| 38 | Human IgA1 | ASPTSPKVFPLSLCSTQPDGNVVIACLVQGFFPQEPLSVTW |
| | heavy chain | SESGQGVTARNFPPSQDASGDLYTTSSQLTLPATQCLAGK |
| | constant | SVTCHVKHYTNPSQDVTVPCPVPSTPPTPSPSTPPTPSPSC |
| | region, e.g., | CHPRLSLHRPALEDLLLGSEANLTCTLTGLRDASGVTFTWT |
| | amino acids | PSSGKSAVQGPPERDLCGCYSVSSVLPGCAEPWNHGKTFT |
| | 144 to 496 | CTAAYPESKTPLTATLSKSGNTFRPEVHLLPPPSEELALNE |
| | of GenBank | LVTLTCLARGFSPKDVLVRWLQGSQELPREKYLTWASRQ |
| | AIC59035.1 | EPSQGTTTFAVTSILRVAAEDWKKGDTFSCMVGHEALPLA |
| | | FTQKTIDRLAGKPTHVNVSVVMAEVDGTCY |
| 39 | Human IgA2 | ASPTSPKVFPLSLDSTPQDGNVVVACLVQGFFPQEPLSVT |
| | heavy chain | WSESGQNVTARNFPPSQDASGDLYTTSSQLTLPATQCPDG |
| | constant | KSVTCHVKHYTNSSQDVTVPCRVPPPPPCCHPRLSLHRPA |
| | region, e.g., | LEDLLLGSEANLTCTLTGLRDASGATFTWTPSSGKSAVQG |
| | amino acids | PPERDLCGCYSVSSVLPGCAQPWNHGETFTCTAAHPELKT |
| | 1 to 340 of | PLTANITKSGNTFRPEVHLLPPPSEELALNELVTLTCLARG |
| | GenBank | FSPKDVLVRWLQGSQELPREKYLTWASRQEPSQGTTTYAV |
| | P01877.4 | TSILRVAAEDWKKGETFSCMVGHEALPLAFTQKTIDRMA |
| | | GKPTHINVSVVMAEADGTCY |
| 40 | Human IgD | APTKAPDVFPIISGCRHPKDNSPVVLACLITGYHPTSVTVT |
| | heavy chain | WYMGTQSQPQRTFPEIQRRDSYYMTSSQLSTPLQQWRQG |
| | constant | EYKCVVQHTASKSKKEIFRWPESPKAQASSVPTAQPQAEG |
| | region, e.g, | SLAKATTAPATTRNTGRGGEEKKKEKEKEEQEERETKTPE |
| | amino acids | CPSHTQPLGVYLLTPAVQDLWLRDKATFTCFVVGSDLKD |
| | 1 to 384 of | AHLTWEVAGKVPTGGVEEGLLERHSNGSQSQHSRLTLPRS |
| | GenBank | LWNAGTSVTCTLNHPSLPPQRLMALREPAAQAPVKLSLNL |
| | P01880.3 | LASSDPPEAASWLLCEVSGFSPPNILLMWLEDQREVNTSG |
| | | FAPARPPPQPRSTTFWAWSVLRVPAPPSPQPATYTCVVSH |
| | | EDSRTLLNASRSLEVSYVTDHGPMK |
| 41 | Human IgE | ASTQSPSVFPLTRCCKNIPSNATSVTLGCLATGYFPEPVMV |
| | heavy chain | TWDTGSLNGTTMTLPATTLTLSGHYATISLLTVSGAWAK |
| | constant | QMFTCRVAHTPSSTDWVDNKTFSVCSRDFTPPTVKILQSS |
| | region, e.g., | CDGGGHFPPTIQLLCLVSGYTPGTINITWLEDGQVMDVDL |
| | amino acids | STASTTQEGELASTQSELTLSQKHWLSDRTYTCQVTYQGH |
| | 1-428 of | TFEDSTKKCADSNPRGVSAYLSRPSPFDLFIRKSPTITCLV |
| | GenBank | VDLAPSKGTVNLTWSRASGKPVNHSTRKEEKQRNGTLTVT |
| | P01854.1 | STLPVGTRDWIEGETYQCRVTHPHLPRALMRSTTKTSGPR |
| | | AAPEVYAFATPEWPGSRDKRTLACLIQNFMPEDISVQWLH |
| | | NEVQLPDARHSTTQPRKTKGSGFFVFSRLEVTRAEWEQKD |
| | | EFICRAVHEAASPSQTVQRAVSVNPGK |
| 42 | mouse IgM | ASQSFPNVFPLVSCESPLSDKNLVAMGCLARDFLPSTISFT |
| | heavy chain | WNYQNNTEVIQGIRTFPTLRTGGKYLATSQVLLSPKSILEG |
| | constant | SDEYLVCKIHYGGKNRDLHVPIPAVAEMNPNVNVFVPPR |
| | region | DGFSGPAPRKSKLICEATNFTPKPITVSWLKDGKLVESGFT |
| | GenBank: | TDPVTIENKGSTPQTYKVISTLTISEIDWLNLNVYTCRVDH |
| | CAC20701.1 | RGLTFLKNVSSTCAASPSTDILNFTIPPSFADIFLSKSANLT |
| | | CLVSNLATYETLSISWASQSGEPLETKIKIMESHPNGTFSAK |
| | | GVASVCVEDWNNRKEFVCTVTHRDLPSPQKKFISKPNEV |
| | | HKHPPAVYLLPPAREQLNLRESATVTCLVKGFSPADISVQ |
| | | WKQRGQLLPQEKYVTSAPMPEPGAPGFYFTHSILTVTEEE |
| | | WNSGETYTCVVGHEALPHLVTERTVDKSTGKPTLYNVSLI |
| | | MSDTGGTCY |
| 43 | Cynomolgus | FWGQGALVTVSSGESAGPFKWEPSVSSPNAPLDTNEVAV |
| | Monkey | GCLAQDFLPDSITFSWKFKNNSDISKGVWGFPSVLRGGKY |
| | presumed IgM | AATSQVLLASKDVMQGTDEHVVCKVQHPNGNKEQNVPL |
| | constant | PVVAERPPNVSVFVPPRDGFVGNPRESKLICQATGFSPRQI |
| | region | EVSWLRDGKQVGSGITTDRVEAEAKESGPTTFKVTSTLTV |
| | sequence | SERDWLSQSVFTCRVDHRGLTFQKNVSSVCGPNPDTAIRV |
| | amino acids | FAIPPSFASIFLTKSTKLTCLVTDLATYDSVTITWTRQNGEA |
| | 14 to 487 | LKTHTNISESHPNGTFSAVGEASICEDDWNSGERFRCTVTH |
| | of Genbank: | TDLPSPLKQTISRPKGVAMHRPDVYLLPPAREQLNLRESAT |
| | EHH62210.1 | ITCLVTGFSPADIFVQWMQRGQPLSPEKYVTSAPMPEPQA |
| | | PGRYFAHSILTVSEEDWNTGETYTCVVAHEALPNRVTERT |
| | | VDKSTGKPTLYNVSLVILWTTLSTFVALFVLTLLYSGIVTFI |
| | | KVR |
| 44 | Chimpanzee | SASAPTLFPLVSCENSPSDTSSVAVGCLAQDFLPDSITFSW |
| | IgM heavy | KYKNNSDISSTRGFPSVLRGGKYAATSQVLLPSKEVMQGT |
| | chain | DEHVVCKVQHPNGNKEKNVPLPVTAELPPKVSIFVPPRDG |
| | constant | FFGNPRSSKLICQATGFSPRQIQVSWLREGKQVGSGVTTD |
| | region | QVQAEAKQSGPTTYKVTSTLTIKESDWLSQSVFTCRVDHR |
| | | GLTFQQNASSMCSPGPDTAIRVFAIPPSFASIFLTKSTKLAC |
| | | LVTDLTTYDSLTISWTRQNGEAVKTHTNISESHPNATFSAV |
| | | GEASICEDDWNSGERFTCTVTHTDLPSPLKQTISRPKEVAL |
| | | HRPDVYLLPPAREQLNLRELATITCLVTGFSPADVFVQWM |
| | | QRGQPLSPEKYVTSAPMPEPQAPGRYFAHSILTVSEEEWN |
| | | TGETYTCVVAHEALPNRVTERTVDKSTGKPTLYNVSLVM |
| | | SDTAGTCY |
| 45 | Rhesus IGM | GSASAPTLFPLVSCENAPLDTNEVAVGCLAQDFLPDSITFS |
| | heavy chain | WKFKNNSDISKGVWGFPSVLRGGKYAATSQVLLASKDV |
| | constant | MQGTDEHVVCKVQHPNGNKEQNVPLPVLAERPPNVSVFV |
| | region (amino | PPRDGFVGNPRESKLICQATGFSPRQIEVSWLREGKQVGSG |
| | acids 147 to | ITTDRVEAEAKESGPTTFKVTSTLTVSERDWLSQSVFTCRV |
| | 600 of | DHRGLTFQKNVSSVCGPNPDTAIRVFAIPPSFASIFLTKSTK |
| | EHH28233.1 | LTCLVTDLATYDSVTITWTRQNGEALKTHTNISESHPNGTF |
| | hypothetical | SAVGEASICEDDWNSGERFRCTVTHTDLPSPLKQTISRPKG |
| | protein | VAMHRPDVYLLPPAREQLNLRESATITCLVTGFSPADIFVQ |
| | EGK_18625 | WMQRGQPLSPEKYVTSAPMPEPQAPGRYFAHSILTVSEED |
| | [Macaca | WNTGETYTCVVAHEALPNRVTERTVDKSTGKPTLYNVSL |
| | mulatta]) | VMSDTAGTCY |
| 46 | Sumatran | GSASAPTLFPLVSCENSLSDTSSVAVGCLAQDFLPDSITFS |
| | orangutan | WKYKNNSDISSTRGFPSVLTGSKYVATSQVLLPSKDVMQ |
| | IgM | GTDEHVVCKVQHPNGNKEKNVPLPVIAELPPKVSIFIPPRD |
| | Constant | GFFGSPRKSKLICQATGFSPRQIQVSWLREGKQVASGITTD |
| | region | QVQAEAKESGPTTYKVTSTLTINESDWLSQSMFTCRVDHR |
| | isoform 2 | GLTFQKNASSMCSPNPNTAIRVFAIPPSFASIFLTKSTKLTC |
| | GenBank: | LVTDLASYDSMTISWTRQNGEAVKTHTNISESHPNATFSA |
| | PNJ04968.1 | VGEASICEDDWNSGERFTCTVTHADLPSPLKQTISRPKGV |
| | | ALHRPDVYLLPPAREQLNLRESATITCLVTGFSPADVFVQ |
| | | WMQRGQPLSPEKYVTSAPMPEPQAPGRYFAHSILTVSEED |
| | | WNTGETYTCVVAHEALPNRVTERTVDKSTGKPTLYNVSL |
| | | VMSDTAGTCY |
| 47 | SP34 VH | EVQLVESGGGLVQPKGSLKLSCAASGFTFNTYAMNWVRQ |
| | | APGKGLEWVARIRSKYNNYATYYADSVKDRFTISRDDSQ |
| | | SILYLQMNNLKTEDTAMYYCVRHGNFGNSYVSWFAYWG |
| | | QGTLVTVSS |
| 48 | SP34 VH CDR1 | GFTFNTYAMN |
| 49 | SP34 VH CDR2 | ARIRSKYNNYATYYADSVKD |
| 50 | SP34 VH CDR3 | VRHGNFGNSYVSWFAY |
| 51 | SP34 VL | QAVVTQESALTTSPGETVTLTCRSSTGAVTTSNYANWVQE |
| | | KPDHLFTGLIGGTNKRAPGVPARFSGSLIGDKAALTITGAQ |
| | | TEDEAIYFCALWYSNLWVFGGGTKLTVL |
| 52 | SP34 VL CDR1 | RSSTGAVTTSNYAN |
| 53 | SP34 VL CDR2 | GTNKRAP |
| 54 | SP34 VL CDR3 | ALWYSNL |
| 55 | SJ* | >SJ* (S = anti CD3 scfv SP34) |
| | | MGWSYIILFLVATATGVHSEVQLVESGGGLVQPKGSLKLS |
| | | CAASGFTFNTYAMNWVRQAPGKGLEWVARIRSKYNNYA |
| | | TYYADSVKDRFTISRDDSQSILYLQMNNLKTEDTAMYYC |
| | | VRHGNFGNSYVSWFAYWGQGTLVTVSSGGGGSGGGGSG |
| | | GGGSQAVVTQESALTTSPGETVTLTCRSSTGAVTTSNYAN |
| | | WVQEKPDHLFTGLIGGTNKRAPGVPARFSGSLIGDKAALT |
| | | ITGAQTEDEAIYFCALWYSNLWVFGGGTKLTVLGGGGSG |
| | | GGGSGGGGSQEDERIVLVDNKCKCARITSRIIRSSEDPNEDI |
| | | VERNIRIIVPLNNRENISDPTSPLRTRFVYHLSDLCKKCDPT |
| | | EVELDNQIVTATQSNICDEDSATETCATYDRNKCYTAVVP |
| | | LVYGGETKMVETALTPDACYPD |
| 56 | N272K; X191 | GSASAPTLFPLVSCENSPSDTSSVAVGCLAQDFLPDSITFS |
| | can be G or S | WKYKNNSDISSTRGFPSVLRGGKYAATSQVLLPSKDVMQ |
| | | GTDEHVVCKVQHPNGNKEKNVPLPVIAELPPKVSVFVPPR |
| | | DGFFGNPRKSKLICQATGFSPRQIQVSWLREGKQVGSGVT |
| | | TDQVQAEAKESGPTTYKVTSTLTIKESDWLXQSMFTCRVD |
| | | HRGLTFQQNASSMCVPDQDTAIRVFAIPPSFASIFLTKSTKL |
| | | TCLVTDLTTYDSVTISWTRQNGEAVKTHTkISESHPNATFS |
| | | AVGEASICEDDWNSGERFTCTVTHTDLPSPLKQTISRPKGV |
| | | ALHRPDVYLLPPAREQLNLRESATITCLVTGFSPADVFVQ |
| | | WMQRGQPLSPEKYVTSAPMPEPQAPGRYFAHSILTVSEEE |
| | | WNTGETYTCVVAHEALPNRVTERTVDKSTGKPTLYNVSL |
| | | VMSDTAGTCY |
| 57 | WO2018208864 | TYAMN |
| 58 | WO2018208864 | DYYMH |
| 59 | WO2018208864 | RIRSKYNNYATYYADSVKD |
| 60 | WO2018208864 | WIDLENANTIYDAKFQG |
| 61 | WO2018208864 | WIDLENANTVYDAKFQG |
| 62 | WO2018208864 | HANFGAGYVSWFAH |
| 63 | WO2018208864 | DAYGRYFYDV |
| 64 | WO2018208864 | DAYGQYFYDV |
| 65 | WO2018208864 | GSSTGAVTTSNYAN |
| 66 | WO2018208864 | KSSQSLLNARTGKNYLA |
| 67 | WO2018208864 | GTDKRAP |
| 68 | WO2018208864 | WASTRES |
| 69 | WO2018208864 | ALWYSNHWV |
| 70 | WO2018208864 | ALWYSDLWV |
| 71 | WO2018208864 | KQSYSRRT |
| 72 | WO2018208864 | KQSYFRRT |
| 73 | WO2018208864 | TQSYFRRT |

TABLE 3
Additional anti-CD3 VH and VL Sequences

| | SEQ | | SEQ | |
|---|---|---|---|---|
| Citation | ID | VH | ID | VL |
| WO2018208864 | 74 | EVQLLESGGGLVQPGGSLRLSCAASGFTFDT | 75 | QTVVTQEPSLSVSPGGTVTLTCGSSTGAVTTS |
| | | YAMNWVRQAPGKGLEWVARIRSKYNNYAT | | NYANWVQQTPGQAPRGLIGGTDKRAPGVPD |
| | | YYADSVKDRFTISRDDSKSTLYLQMESLRAE | | RFSGSLLGDKAALTITGAQAEDEADYYCALW |
| | | DTAVYYCVRHANFGAGYVSWFAHWGQGTL | | YSNHWVFGGGTKLTVL |
| | | VTVSS | | |
| WO2018208864 | 76 | EVQLLESGGGLVQPGGSLRLSCAASGFTFDT | 77 | QTVVTQEPSLSVSPGGTVTLTCGSSTGAVTTS |
| | | YAMNWVRQAPGKGLEWVARIRSKYNNYAT | | NYANWVQQTPGQAPRGLIGGTDKRAPGVPD |
| | | YYADSVKDRFTISRDDSKSTLYLQMESLRAE | | RFSGSLLGDKAALTITGAQAEDEADYYCALW |
| | | DTAVYYCVRHANFGAGYVSWFAHWGQGTL | | YSDLWVFGGGTKLTVL |
| | | VTVSS | | |
| WO2018208864 | 78 | QVQLVQSGAEVKKPGASVKVSCKASGFNIK | 79 | DIVMTQSPDSLAVSLGERATINCKSSQSLLNA |
| | | DYYMHWVRQAPGQRLEWMGWIDLENANTI | | RTGKNYLAWYQQKPGQPPKLLIYWASTRESG |
| | | YDAKFQGRVTITRDTSASTAYMELSSLRSED | | VPDRFSGSGSGTDFTLTISSLQAEDVAVYYCK |
| | | TAVYYCARDAYGRYFYDVWGQGTLVTVSS | | QSYSRRTFGGGTKVEIK |
| WO2018208864 | 80 | QVQLVQSGAEVKKPGASVKVSCKASGFNIK | 81 | DIVMTQSPDSLAVSLGERATINCKSSQSLLNA |
| | | DYYMHWVRQAPGQRLEWIGWIDLENANTV | | RTGKNYLAWYQQKPGQPPKLLIYWASTRESG |
| | | YDAKFQGRVTITRDTSASTAYMELSSLRSED | | VPDRFSGSGSGTDFTLTISSLQAEDVAVYYCK |
| | | TAVYYCARDAYGRYFYDVWGQGTLVTVSS | | QSYFRRTFGGGTKVEIK |
| WO2018208864 | 82 | QVQLVQSGAEVKKPGASVKVSCKASGFNIK | 83 | DIVMTQSPDSLAVSLGERATINCKSSQSLLNA |
| | | DYYMHWVRQAPGQRLEWIGWIDLENANTV | | RTGKNYLAWYQQKPGQPPKLLIYWASTRESG |
| | | YDAKFQGRVTITRDTSASTAYMELSSLRSED | | VPDRFSGSGSGTDFTLTISSLQAEDVAVYYCT |
| | | TAVYYCARDAYGQYFYDVWGQGTLVTVSS | | QSYFRRTFGGGTKVEIK |

What is claimed is:

1. An isolated IgM-derived binding molecule comprising at least one variant IgM-derived heavy chain, wherein the at least one variant IgM-derived heavy chain comprises a variant IgM heavy chain constant region associated with a binding domain that specifically binds to a target, wherein at least one asparagine(N)-linked glycosylation motif of the variant IgM heavy chain constant region is mutated to prevent glycosylation at that motif, and wherein an N-linked glycosylation motif comprises the amino acid sequence N-X₁-S/T, wherein N is asparagine, X₁ is any amino acid except proline, and S/T is serine or threonine.

2. The IgM-derived binding molecule of claim 1, wherein the variant IgM heavy chain constant region is derived from a human IgM heavy chain constant region comprising five N-linked glycosylation motifs N-X₁-S/T starting at amino acid positions corresponding to amino acid 46 (motif N1), amino acid 209 (motif N2), amino acid 272 (motif N3), amino acid 279 (motif N4), and amino acid 440 (motif N5) of SEQ ID NO: 1 (allele IGHM*03) or SEQ ID NO: 2 (allele IGHM*04).

3. The IgM-derived binding molecule of claim 2, wherein at least one, at least two, at least three, or at least four of the N-X₁-S/T motifs comprises an amino acid insertion, deletion, or substitution that prevents glycosylation at that motif.

4. The IgM-derived binding molecule of claim 3, comprising an amino acid insertion, deletion, or substitution at motif N1, motif N2, motif N3, motif N5, or any combination of two or more, three or more, or all four of motifs N1, N2, N3, or N5, wherein the amino acid insertion, deletion, or substitution prevents glycosylation at that motif.

5. The IgM-derived binding molecule of claim 4, comprising an amino acid substitution at an amino acid position corresponding to amino acid N46, N209, N272, or N440 of SEQ ID NO: 1 or SEQ ID NO: 2 wherein the substituted amino acid is any amino acid, an amino acid substitution at an amino acid position corresponding to amino acid S48, S211, S274, or S442 of SEQ ID NO: 1 or SEQ ID NO: 2 wherein the substituted amino acid is any amino acid except threonine, or any combination of two or more, three or more, or four or more of the amino acid substitutions.

6. The IgM-derived binding molecule of claim 5, comprising an amino acid substitution corresponding to N46X₂, N46A, N46D, N46Q, N46K, 548X₃, S48A, N229X₂, N229A, N229D, N229Q, N229K, S231X₃, S231A, N272X₂, N272A, N272D, N272Q, N272K, S274X₃, S274A, N440X₂, N440A, N440D, N449Q, N449K, S242X₃, or S424A of SEQ ID NO: 1 or SEQ ID NO: 2, or any combination of two or more, three or more, or four or more of the amino acid substitutions, wherein X₂ is any amino acid and X₃ is any amino acid except threonine.

7. The IgM-derived binding molecule of any one of claims 1 to 6, wherein the variant IgM heavy chain constant region is a variant human IgM constant region comprising the amino acid sequence SEQ ID NO: 3, SEQ ID NO: 4, SEQ ID NO: 5, SEQ ID NO: 6, SEQ ID NO: 7, SEQ ID NO: 8, SEQ ID NO: 11, SEQ ID NO: 12, SEQ ID NO: 13, SEQ ID NO: 14, SEQ ID NO: 15, SEQ ID NO: 16, SEQ ID NO: 17, or SEQ ID NO: 18.

8. The IgM-derived binding molecule of any one of claims 1 to 7, wherein the variant IgM heavy chain constant region is mutated to introduce at least one new asparagine(N)-linked glycosylation motif into the variant IgM heavy chain constant region, wherein the at least one new asparagine(N)-linked glycosylation motif is introduced at a site in the variant IgM heavy chain constant region that is not naturally glycosylated in an IgM antibody.

9. The IgM-derived binding molecule of claim 8, wherein the new asparagine(N)-linked glycosylation motif is at a position in the variant IgM heavy chain constant region that corresponds to the position of an asparagine(N)-linked glycosylation motif present in a different immunoglobulin isotype.

10. The IgM-derived binding molecule of claim 9, wherein the different immunoglobulin isotype is a human immunoglobulin isotype selected from the group consisting of human IgG1, human IgG2, human IgG3, human IgG4, human IgA1, human IgA2, human IgD, and human IgE.

11. The IgM-derived binding molecule of any one of claims 1 to 10, wherein the target is a target epitope, a target antigen, a target cell, a target organ, or a target virus.

12. The IgM-derived binding molecule of any one of claims 1 to 11, which is a pentameric or a hexameric IgM antibody comprising five or six bivalent IgM binding units, respectively, wherein each binding unit comprises two IgM heavy chains each comprising a VH situated amino terminal to the variant IgM constant region, and two immunoglobulin light chains each comprising a light chain variable domain (VL) situated amino terminal to an immunoglobulin light chain constant region, and wherein the VH and VL combine to form an antigen-binding domain that specifically binds to the target.

13. The IgM-derived binding molecule of claim 12, wherein the five or six IgM binding units are identical.

14. The IgM-derived binding molecule of claim 12 or claim 13, which is pentameric, and further comprises a J-chain, or functional fragment thereof, or a functional variant thereof.

15. The IgM-derived binding molecule of claim 14, wherein the J-chain is a mature human J-chain comprising the amino acid sequence SEQ ID NO: 20 or a functional fragment thereof, or a functional variant thereof.

16. The IgM-derived binding molecule of claim 14 or claim 15, wherein the J-chain is a functional variant J-chain comprising one or more single amino acid substitutions, deletions, or insertions relative to a reference J-chain identical to the variant J-chain except for the one or more single amino acid substitutions, deletions, or insertions, and wherein the IgM-derived binding molecule exhibits an increased serum half-life upon administration to a subject animal relative to a reference IgM-derived binding molecule that is identical except for the one or more single amino acid substitutions, deletions, or insertions in the variant J-chain, and is administered in the same way to the same animal species.

17. The IgM-derived binding molecule of claim 16, wherein the variant J-chain or functional fragment thereof comprises one, two, three, or four single amino acid substitutions, deletions, or insertions relative to the reference J-chain.

18. The IgM-derived binding molecule of claim 16 or claim 17, wherein the variant J-chain or functional fragment thereof comprises an amino acid substitution at the amino acid position corresponding to amino acid Y102 of the wild-type mature human J-chain (SEQ ID NO: 20).

19. The IgM-derived binding molecule of claim 18, wherein the amino acid corresponding to Y102 of SEQ ID NO: 20 is substituted with alanine (A).

20. The IgM-derived binding molecule of claim 19, wherein the J-chain is the variant human J-chain J*, which comprises the amino acid sequence SEQ ID NO: 21.

21. The IgM-derived binding molecule of any one of claims 16 to 19, wherein the variant J-chain or functional fragment thereof comprises an a mutation within the asparagine(N)-linked glycosylation motif N-X₁-S/T starting at the amino acid position corresponding to amino acid 49 (motif N6) of the mature human J-chain (SEQ ID NO: 20), wherein N is asparagine, X₁ is any amino acid except proline, and S/T is serine or threonine, and wherein the mutation prevents glycosylation at that motif.

22. The IgM-derived binding molecule of claim 21, wherein the variant J-chain or functional fragment thereof comprises an amino acid substation at the amino acid position corresponding to amino acid N49 or amino acid S51 SEQ ID NO: 20 wherein the amino acid corresponding to S51 is not substituted with threonine (T), or wherein the variant J-chain comprises amino acid substitutions at the amino acid positions corresponding to both amino acids N49 and S51 of SEQ ID NO: 20.

23. The IgM-derived binding molecule of claim 22, wherein the position corresponding to N49 of SEQ ID NO: 20 is substituted with alanine (A), glycine (G), threonine (T), serine (S) or aspartic acid (D).

24. The IgM-derived binding molecule of claim 23, wherein the position corresponding to N49 of SEQ ID NO: 20 is substituted with alanine (A).

25. The IgM-derived binding molecule of claim 24, wherein the J-chain is a variant human J-chain and comprises the amino acid sequence SEQ ID NO: 22.

26. The IgM-derived binding molecule of claim 23, wherein the position corresponding to N49 of SEQ ID NO: 20 is substituted with aspartic acid (D).

27. The IgM-derived binding molecule of claim 26, wherein the J-chain is a variant human J-chain and comprises the amino acid sequence SEQ ID NO: 23.

28. The IgM-derived binding molecule of any one of claims 14 to 27, wherein the J-chain or fragment or variant thereof is a modified J-chain further comprising a heterologous moiety, wherein the heterologous moiety is fused or conjugated to the J-chain or fragment or variant thereof.

29. The IgM-derived binding molecule of claim 28, wherein the heterologous moiety is a polypeptide fused to the J-chain or fragment or variant thereof.

30. The IgM-derived binding molecule of claim 29, wherein the heterologous polypeptide is fused to the J-chain or fragment or variant thereof via a peptide linker.

31. The IgM-derived binding molecule of claim 30, wherein the peptide linker comprises at least 5 amino acids, but no more than 25 amino acids.

32. The IgM-derived binding molecule of claim 30 or claim 31, wherein the peptide linker consists of GGGGSGGGGSGGGGS (SEQ ID NO: 29).

33. The IgM-derived binding molecule of any one of claims 29 to 32, wherein the heterologous polypeptide is fused to the N-terminus of the J-chain or fragment or variant thereof, the C-terminus of the J-chain or fragment or variant thereof, or to both the N-terminus and C-terminus of the J-chain or fragment or variant thereof.

34. The IgM-derived binding molecule of any one of claims 29 to 33, wherein the heterologous polypeptide comprises a binding domain.

35. The IgM-derived binding molecule of claim 34, wherein the binding domain of the heterologous polypeptide is an antibody or antigen-binding fragment thereof.

36. The IgM-derived binding molecule of claim 35, wherein the antigen-binding fragment is a scFv fragment.

37. The IgM-derived binding molecule of claim 36, wherein the heterologous scFv fragment specifically binds to CD3c.

38. The IgM-derived binding molecule of claim 37, wherein the modified J-chain comprises the amino acid sequence SEQ ID NO: 24 (V15J), SEQ ID NO: 25 (V15J*), SEQ ID NO: 26 (V15J N49D), or SEQ ID NO: 55 (SP) or SEQ ID NOs: 20, 21, 22, or 23 fused via a peptide linker to an anti-CD3c scFv comprising HCDR1, HCDR2, HCDR3, LCDR1, LCDR2, and LCDR3 amino acid sequences comprising SEQ ID NO: 48, SEQ ID NO: 49, SEQ ID NO: 50, SEQ ID NO: 52, SEQ ID NO: 53, and SEQ ID NO: 54; SEQ ID NO: 57, SEQ ID NO: 59, SEQ ID NO: 62, SEQ ID NO: 65, SEQ ID NO: 67, and SEQ ID NO: 69; SEQ ID NO: 57, SEQ ID NO: 59, SEQ ID NO: 62, SEQ ID NO: 65, SEQ ID NO: 67, and SEQ ID NO: 70; SEQ ID NO: 58, SEQ ID NO: 60, SEQ ID NO: 63, SEQ ID NO: 66, SEQ ID NO: 68, and SEQ ID NO: 71; SEQ ID NO: 58, SEQ ID NO: 61, SEQ ID NO: 63, SEQ ID NO: 66, SEQ ID NO: 68, and SEQ ID NO: 72; SEQ ID NO: 58, SEQ ID NO: 61, SEQ ID NO: 64, SEQ ID NO: 66, SEQ ID NO: 68, and SEQ ID NO: 73, respectively.

39. A polynucleotide comprising a nucleic acid sequence that encodes the at least one variant IgM-derived heavy chain of any one of claims 1 to 37.

40. A composition comprising the polynucleotide of claim 39.

41. The composition of claim 40, further comprising a nucleic acid sequence that encodes a light chain polypeptide subunit.

42. The composition of claim 41, wherein the nucleic acid sequence encoding the at least one variant IgM-derived heavy chain and the nucleic acid sequence encoding the light chain polypeptide subunit are on separate vectors.

43. The composition of claim 41, wherein the nucleic acid sequence encoding the at least one variant IgM-derived heavy chain and the nucleic acid sequence encoding the light chain polypeptide subunit are on a single vector.

44. The composition of any one of claims 41 to 43, further comprising a nucleic acid sequence that encodes a J-chain, or functional fragment thereof, or a functional variant thereof.

45. The composition of claim 44, wherein the nucleic acid sequence encoding the at least one variant IgM-derived heavy chain, the nucleic acid sequence encoding the light chain polypeptide subunit, and the nucleic acid sequence encoding the J-chain are on a single vector.

46. The composition of claim 44, wherein the nucleic acid sequence encoding the at least one variant IgM-derived heavy chain, the nucleic acid sequence encoding the light chain polypeptide subunit, and the nucleic acid sequence encoding the J-chain are each on separate vectors.

47. The vector or vectors of any one of claim 42, 43, 45, or 46.

48. A host cell comprising the polynucleotide of claim 39, the composition of any one of claims 40 to 46, or the vector or vectors of claim 47, wherein the host cell can express the IgM-derived binding molecule of any one of claims 1 to 37.

49. A method of producing the IgM-derived binding molecule of any one of claims 1 to 37, comprising culturing the host cell of claim 48, and recovering the constant region or antibody.


SEQUENCE LISTING (84 sequences)

SEQ ID NO: 1 - protein, 453 aa, Homo sapiens [sequence omitted]

SEQ ID NO: 2 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 3 - protein, 453 aa, Artificial Sequence
  Description of Artificial Sequence Synthetic polypeptide
  MOD_RES (191)..(191): Gly or Ser
GSASAPTLFPLVSCENSPSDTSSVAVGCLAQDFLPDSITFSWKYKANSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLXQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 4 - protein, 453 aa, Artificial Sequence
  Description of Artificial Sequence Synthetic polypeptide
  MOD_RES (191)..(191): Gly or Ser
GSASAPTLFPLVSCENSPSDTSSVAVGCLAQDFLPDSITFSWKYKDNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLXQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 5 - protein, 453 aa, Artificial Sequence
  Description of Artificial Sequence Synthetic polypeptide
  MOD_RES (191)..(191): Gly or Ser
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLXQSMFTCRVDHRGLTFQQAASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 6 - protein, 453 aa, Artificial Sequence
  Description of Artificial Sequence Synthetic polypeptide
  MOD_RES (191)..(191): Gly or Ser
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLXQSMFTCRVDHRGLTFQQDASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 7 - protein, 453 aa, Artificial Sequence
  Description of Artificial Sequence Synthetic polypeptide
  MOD_RES (191)..(191): Gly or Ser
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLXQSMFTCRVDHRGLTFQQNASSMCVPDQDTAIRVFAIPPSFASIFLTKSTKLTCLVTDLTTYDSVTISWTRQNGEAVKTHTAISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 8 - protein, 453 aa, Artificial Sequence
  Description of Artificial Sequence Synthetic polypeptide
  MOD_RES (191)..(191): Gly or Ser
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLXQSMFTCRVDHRGLTFQQNASSMCVPDQDTAIRVFAIPPSFASIFLTKSTKLTCLVTDLTTYDSVTISWTRQNGEAVKTHTDISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 9 - protein, 453 aa, Artificial Sequence
  Description of Artificial Sequence Synthetic polypeptide
  MOD_RES (191)..(191): Gly or Ser
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLXQSMFTCRVDHRGLTFQQNASSMCVPDQDTAIRVFAIPPSFASIFLTKSTKLTCLVTDLTTYDSVTISWTRQNGEAVKTHTNISESHPAATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 10 - protein, 453 aa, Artificial Sequence
  Description of Artificial Sequence Synthetic polypeptide
  MOD_RES (191)..(191): Gly or Ser
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLXQSMFTCRVDHRGLTFQQNASSMCVPDQDTAIRVFAIPPSFASIFLTKSTKLTCLVTDLTTYDSVTISWTRQNGEAVKTHTNISESHPDATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 11 - protein, 453 aa, Artificial Sequence
  Description of Artificial Sequence Synthetic polypeptide
  MOD_RES (191)..(191): Gly or Ser
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLXQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYAVSLVMSDTAGTCY

SEQ ID NO: 12 - protein, 453 aa, Artificial Sequence
  Description of Artificial Sequence Synthetic polypeptide
  MOD_RES (191)..(191): Gly or Ser
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLXQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYDVSLVMSDTAGTCY

SEQ ID NO: 13 - protein, 453 aa, Artificial Sequence
  Description of Artificial Sequence Synthetic polypeptide
  MOD_RES (191)..(191): Gly or Ser
GSASAPTLFPLVSCENSPSDTSSVAVGCLAQDFLPDSITFSWKYKDNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLXQSMFTCRVDHRGLTFQQDASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 14 - protein, 453 aa, Artificial Sequence
  Description of Artificial Sequence Synthetic polypeptide
  MOD_RES (191)..(191): Gly or Ser
GSASAPTLFPLVSCENSPSDTSSVAVGCLAQDFLPDSITFSWKYKDNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLXQSMFTCRVDHRGLTFQQDASSMCVPDQDTAIRVFAIPPSFASIFLTKSTKLTCLVTDLTTYDSVTISWTRQNGEAVKTHTDISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 15 - protein, 453 aa, Artificial Sequence
  Description of Artificial Sequence Synthetic polypeptide
  MOD_RES (191)..(191): Gly or Ser
GSASAPTLFPLVSCENSPSDTSSVAVGCLAQDFLPDSITFSWKYKDNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLXQSMFTCRVDHRGLTFQQNASSMCVPDQDTAIRVFAIPPSFASIFLTKSTKLTCLVTDLTTYDSVTISWTRQNGEAVKTHTDISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 16 - protein, 453 aa, Artificial Sequence
  Description of Artificial Sequence Synthetic polypeptide
  MOD_RES (191)..(191): Gly or Ser
GSASAPTLFPLVSCENSPSDTSSVAVGCLAQDFLPDSITFSWKYKDNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLXQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYDVSLVMSDTAGTCY

SEQ ID NO: 17 - protein, 453 aa, Artificial Sequence; identical to SEQ ID NO: 14
  Description of Artificial Sequence Synthetic polypeptide
  MOD_RES (191)..(191): Gly or Ser

SEQ ID NO: 18 - protein, 453 aa, Artificial Sequence
  Description of Artificial Sequence Synthetic polypeptide
  MOD_RES (191)..(191): Gly or Ser
GSASAPTLFPLVSCENSPSDTSSVAVGCLAQDFLPDSITFSWKYKDNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLXQSMFTCRVDHRGLTFQQDASSMCVPDQDTAIRVFAIPPSFASIFLTKSTKLTCLVTDLTTYDSVTISWTRQNGEAVKTHTDISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYDVSLVMSDTAGTCY

SEQ ID NO: 19 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 20 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 21 - protein, 137 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 22 - protein, 137 aa, Artificial Sequence
  Description of Artificial Sequence Synthetic polypeptide
QEDERIVLVDNKCKCARITSRIIRSSEDPNEDIVERNIRIIVPLNNREAISDPTSPLRTRFVYHLSDLCKKCDPTEVELDNQIVTATQSNICDEDSATETCYTYDRNKCYTAVVPLVYGGETKMVETALTPDACYPD

SEQ ID NO: 23 - protein, 137 aa, Artificial Sequence
  Description of Artificial Sequence Synthetic polypeptide
QEDERIVLVDNKCKCARITSRIIRSSEDPNEDIVERNIRIIVPLNNREDISDPTSPLRTRFVYHLSDLCKKCDPTEVELDNQIVTATQSNICDEDSATETCYTYDRNKCYTAVVPLVYGGETKMVETALTPDACYPD

SEQ ID NO: 24 - protein, 393 aa, Artificial Sequence
  Description of Artificial Sequence Synthetic polypeptide
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKLEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 25 - protein, 393 aa, Artificial Sequence
  Description of Artificial Sequence Synthetic polypeptide
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKLEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 26 - protein, 393 aa, Artificial Sequence
  Description of Artificial Sequence Synthetic polypeptide
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKLEIKGGGGSGGGGSGGGGSQEDERIVLVDNKCKCARITSRIIRSSEDPNEDIVERNIRIIVPLNNREDISDPTSPLRTRFVYHLSDLCKKCDPTEVELDNQIVTATQSNICDEDSATETCYTYDRNKCYTAVVPLVYGGETKMVETALTPDACYPD

SEQ ID NO: 27 - protein, 5 aa, Artificial Sequence
  Description of Artificial Sequence Synthetic peptide
GGGGS

SEQ ID NO: 28 - protein, 10 aa, Artificial Sequence
  Description of Artificial Sequence Synthetic peptide
GGGGSGGGGS

SEQ ID NO: 29 - protein, 15 aa, Artificial Sequence
  Description of Artificial Sequence Synthetic peptide
GGGGSGGGGSGGGGS

SEQ ID NO: 30 - protein, 20 aa, Artificial Sequence
  Description of Artificial Sequence Synthetic peptide
GGGGSGGGGSGGGGSGGGGS

SEQ ID NO: 31 - protein, 25 aa, Artificial Sequence
  Description of Artificial Sequence Synthetic peptide
GGGGSGGGGSGGGGSGGGGSGGGGS

SEQ ID NO: 32 - protein, 122 aa, Artificial Sequence
  Description of Artificial Sequence Synthetic polypeptide
EVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSS

SEQ ID NO: 33 - protein, 112 aa, Artificial Sequence
  Description of Artificial Sequence Synthetic polypeptide
DIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIK

SEQ ID NO: 34 - protein, 330 aa, Homo sapiens [sequence omitted]

SEQ ID NO: 35 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 36 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQFNSTFRVVSVLTVLHQDWLNGKEYKCKVSNKGLPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 37 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 39 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 40 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 41 - protein, 428 aa, Homo sapiens
ASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 42 - protein, 455 aa, Mus musculus
ASQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNRDLHVPIPAVAEMNPNVNVFVPPRDGFSGPAPRKSKLICEATNFTPKPITVSWLKDGKLVESGFTTDPVTIENKGSTPQTYKVISTLTISEIDWLNLNVYTCRVDHRGLTFLKNVSSTCAASPSTDILNFTIPPSFADIFLSKSANLTCLVSNLATYETLSISWASQSGEPLETKIKIMESHPNGTFSAKGVASVCVEDWNNRKEFVCTVTHRDLPSPQKKFISKPNEVHKHPPAVYLLPPAREQLNLRESATVTCLVKGFSPADISVQWKQRGQLLPQEKYVTSAPMPEPGAPGFYFTHSILTVTEEEWNSGETYTCVVGHEALPHLVTERTVDKSTGKPTLYNVSLIMSDTGGTCY

SEQ ID NO: 43 - protein, 487 aa, Macaca fascicularis
FWGQGALVTVSSGESAGPFKWEPSVSSPNAPLDTNEVAVGCLAQDFLPDSITFSWKFKNNSDISKGVWGFPSVLRGGKYAATSQVLLASKDVMQGTDEHVVCKVQHPNGNKEQNVPLPVVAERPPNVSVFVPPRDGFVGNPRESKLICQATGFSPRQIEVSWLRDGKQVGSGITTDRVEAEAKESGPTTFKVTSTLTVSERDWLSQSVFTCRVDHRGLTFQKNVSSVCGPNPDTAIRVFAIPPSFASIFLTKSTKLTCLVTDLATYDSVTITWTRQNGEALKTHTNISESHPNGTFSAVGEASICEDDWNSGERFRCTVTHTDLPSPLKQTISRPKGVAMHRPDVYLLPPAREQLNLRESATITCLVTGFSPADIFVQWMQRGQPLSPEKYVTSAPMPEPQAPGRYFAHSILTVSEEDWNTGETYTCVVAHEALPNRVTERTVDKSTGKPTLYNVSLVILWTTLSTFVALFVLTLLYSGIVTFIKVR

SEQ ID NO: 44 - protein, 452 aa, Pan troglodytes
SASAPTLFPLVSCENSPSDTSSVAVGCLAQDFLPDSITFSWKYKNNSDISSTRGFPSVLRGGKYAATSQVLLPSKEVMQGTDEHVVCKVQHPNGNKEKNVPLPVTAELPPKVSIFVPPRDGFFGNPRSSKLICQATGFSPRQIQVSWLREGKQVGSGVTTDQVQAEAKQSGPTTYKVTSTLTIKESDWLSQSVFTCRVDHRGLTFQQNASSMCSPGPDTAIRVFAIPPSFASIFLTKSTKLACLVTDLTTYDSLTISWTRQNGEAVKTHTNISESHPNATFSAVGEASICEDDWNSGERFTCTVTHTDLPSPLKQTISRPKEVALHRPDVYLLPPAREQLNLRELATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 45 - protein, 454 aa, Macaca mulatta
GSASAPTLFPLVSCENAPLDTNEVAVGCLAQDFLPDSITFSWKFKNNSDISKGVWGFPSVLRGGKYAATSQVLLASKDVMQGTDEHVVCKVQHPNGNKEQNVPLPVLAERPPNVSVFVPPRDGFVGNPRESKLICQATGFSPRQIEVSWLREGKQVGSGITTDRVEAEAKESGPTTFKVTSTLTVSERDWLSQSVFTCRVDHRGLTFQKNVSSVCGPNPDTAIRVFAIPPSFASIFLTKSTKLTCLVTDLATYDSVTITWTRQNGEALKTHTNISESHPNGTFSAVGEASICEDDWNSGERFRCTVTHTDLPSPLKQTISRPKGVAMHRPDVYLLPPAREQLNLRESATITCLVTGFSPADIFVQWMQRGQPLSPEKYVTSAPMPEPQAPGRYFAHSILTVSEEDWNTGETYTCVVAHEALPNRVTERTVDKSTGKPTLYNVSLVMSDTAGTCY

SEQ ID NO: 46 - protein, 453 aa, Pongo abelii
GSASAPTLFPLVSCENSLSDTSSVAVGCLAQDFLPDSITFSWKYKNNSDISSTRGFPSVLTGSKYVATSQVLLPSKDVMQGTDEHVVCKVQHPNGNKEKNVPLPVIAELPPKVSIFIPPRDGFFGSPRKSKLICQATGFSPRQIQVSWLREGKQVASGITTDQVQAEAKESGPTTYKVTSTLTINESDWLSQSMFTCRVDHRGLTFQKNASSMCSPNPNTAIRVFAIPPSFASIFLTKSTKLTCLVTDLASYDSMTISWTRQNGEAVKTHTNISESHPNATFSAVGEASICEDDWNSGERFTCTVTHADLPSPLKQTISRPKGVALHRPDVYLLPPAREQLNLRESATITCLVTGFSPADVFVQWMQRGQPLSPEKYVTSAPMPEPQAPGRYFAHSILTVSEEDWNTGETYTCVVAHEALPNRVTERTVDKSTGKPTLYNVSLVMSDTAGTCY

SEQ ID NO: 47 - protein, 125 aa, Artificial Sequence
  Description of Artificial Sequence Synthetic polypeptide
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSS

SEQ ID NO: 48 - protein, 10 aa, Artificial Sequence
  Description of Artificial Sequence Synthetic peptide
GFTFNTYAMN

SEQ ID NO: 49 - protein, 20 aa, Artificial Sequence
  Description of Artificial Sequence Synthetic peptide
ARIRSKYNNYATYYADSVKD

SEQ ID NO: 50 - protein, 16 aa, Artificial Sequence
  Description of Artificial Sequence Synthetic peptide
VRHGNFGNSYVSWFAY

SEQ ID NO: 51 - protein, 109 aa, Artificial Sequence
  Description of Artificial Sequence Synthetic polypeptide
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 52 - protein, 14 aa, Artificial Sequence
  Description of Artificial Sequence Synthetic peptide
RSSTGAVTTSNYAN

SEQ ID NO: 53 - protein, 7 aa, Artificial Sequence
  Description of Artificial Sequence Synthetic peptide
GTNKRAP

SEQ ID NO: 54 - protein, 7 aa, Artificial Sequence
  Description of Artificial Sequence Synthetic peptide
ALWYSNL

SEQ ID NO: 55 - protein, 420 aa, Artificial Sequence
  Description of Artificial Sequence Synthetic polypeptide
MGWSYIILFLVATATGVHSEVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 56 - protein, 453 aa, Artificial Sequence
  Description of Artificial Sequence Synthetic polypeptide
  MOD_RES (191)..(191): Gly or Ser
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLXQSMFTCRVDHRGLTFQQNASSMCVPDQDTAIRVFAIPPSFASIFLTKSTKLTCLVTDLTTYDSVTISWTRQNGEAVKTHTKISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 57 - protein, 5 aa, Artificial Sequence
  Description of Artificial Sequence Synthetic peptide
TYAMN

SEQ ID NO: 58 - protein, 5 aa, Artificial Sequence
  Description of Artificial Sequence Synthetic peptide
DYYMH

SEQ ID NO: 59 - protein, 19 aa, Artificial Sequence
  Description of Artificial Sequence Synthetic peptide
RIRSKYNNYATYYADSVKD

SEQ ID NO: 60 - protein, 17 aa, Artificial Sequence
  Description of Artificial Sequence Synthetic peptide
WIDLENANTIYDAKFQG

SEQ ID NO: 61 - protein, 17 aa, Artificial Sequence
  Description of Artificial Sequence Synthetic peptide
WIDLENANTVYDAKFQG

SEQ ID NO: 62 - protein, 14 aa, Artificial Sequence
  Description of Artificial Sequence Synthetic peptide
HANFGAGYVSWFAH

SEQ ID NO: 63 - protein, 10 aa, Artificial Sequence
  Description of Artificial Sequence Synthetic peptide
DAYGRYFYDV

SEQ ID NO: 64 - protein, 10 aa, Artificial Sequence
  Description of Artificial Sequence Synthetic peptide
DAYGQYFYDV

SEQ ID NO: 65 - protein, 14 aa, Artificial Sequence
  Description of Artificial Sequence Synthetic peptide
GSSTGAVTTSNYAN

SEQ ID NO: 66 - protein, 17 aa, Artificial Sequence
  Description of Artificial Sequence Synthetic peptide
KSSQSLLNARTGKNYLA

SEQ ID NO: 67 - protein, 7 aa, Artificial Sequence
  Description of Artificial Sequence Synthetic peptide
GTDKRAP

SEQ ID NO: 68 - protein, 7 aa, Artificial Sequence
  Description of Artificial Sequence Synthetic peptide
WASTRES

SEQ ID NO: 69 - protein, 9 aa, Artificial Sequence
  Description of Artificial Sequence Synthetic peptide
ALWYSNHWV

SEQ ID NO: 70 - protein, 9 aa, Artificial Sequence
  Description of Artificial Sequence Synthetic peptide
ALWYSDLWV

SEQ ID NO: 71 - protein, 8 aa, Artificial Sequence
  Description of Artificial Sequence Synthetic peptide
KQSYSRRT

SEQ ID NO: 72 - protein, 8 aa, Artificial Sequence
  Description of Artificial Sequence Synthetic peptide
KQSYFRRT

SEQ ID NO: 73 - protein, 8 aa, Artificial Sequence
  Description of Artificial Sequence Synthetic peptide
TQSYFRRT

SEQ ID NO: 74 - protein, 125 aa, Artificial Sequence
  Description of Artificial Sequence Synthetic polypeptide
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSS

SEQ ID NO: 75 - protein, 109 aa, Artificial Sequence
  Description of Artificial Sequence Synthetic polypeptide
QTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVL

SEQ ID NO: 76 - protein, 125 aa, Artificial Sequence; identical to SEQ ID NO: 74
  Description of Artificial Sequence Synthetic polypeptide

SEQ ID NO: 77 - protein, 109 aa, Artificial Sequence
  Description of Artificial Sequence Synthetic polypeptide
QTVVTQEPSLSVSPGGTVTLTCGSSTGAVTTSNYANWVQQTPGQAPRGLIGGTDKRAPGVPDRFSGSLLGDKAALTITGAQAEDEADYYCALWYSDLWVFGGGTKLTVL

SEQ ID NO: 78 - protein, 119 aa, Artificial Sequence
  Description of Artificial Sequence Synthetic polypeptide
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSS

SEQ ID NO: 79 - protein, 112 aa, Artificial Sequence
  Description of Artificial Sequence Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIK

SEQ ID NO: 80 - protein, 119 aa, Artificial Sequence
  Description of Artificial Sequence Synthetic polypeptide
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGRYFYDVWGQGTLVTVSS

SEQ ID NO: 81 - protein, 112 aa, Artificial Sequence
  Description of Artificial Sequence Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYFRRTFGGGTKVEIK

SEQ ID NO: 82 - protein, 119 aa, Artificial Sequence
  Description of Artificial Sequence Synthetic polypeptide
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGQYFYDVWGQGTLVTVSS

SEQ ID NO: 83 - protein, 112 aa, Artificial Sequence
  Description of Artificial Sequence Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCTQSYFRRTFGGGTKVEIK

SEQ ID NO: 84 - protein, 25 aa, Artificial Sequence
  Description of Artificial Sequence Synthetic peptide
  MISC_FEATURE (1)..(25): This sequence may encompass 1-5 “Gly Gly Gly Gly Ser” repeating units
  See specification as filed for detailed description of substitutions and preferred embodiments
GGGGSGGGGSGGGGSGGGGSGGGGS